SEQUENCE LISTING (4 sequences)

SEQ ID NO: 1 - DNA, 29903 bp, Severe acute respiratory syndrome-related coronavirus
  source 1..29903: mol_type = genomic DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 2 - DNA, 29882 bp, Severe acute respiratory syndrome-related coronavirus
  source 1..29882: mol_type = genomic DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactttttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaa

SEQ ID NO: 3 - DNA, 29751 bp, Severe acute respiratory syndrome-related coronavirus
  source 1..29751: mol_type = genomic DNA
atattaggtttttacctacccaggaaaagccaaccaacctcgatctcttgtagatctgttctctaaacgaactttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaagatggagagccttgttcttggtgtcaacgagaaaacacacgtccaactcagtttgcctgtccttcaggttagagacgtgctagtgcgtggcttcggggactctgtggaagaggccctatcggaggcacgtgaacacctcaaaaatggcacttgtggtctagtagagctggaaaaaggcgtactgccccagcttgaacagccctatgtgttcattaaacgttctgatgccttaagcaccaatcacggccacaaggtcgttgagctggttgcagaaatggacggcattcagtacggtcgtagcggtataacactgggagtactcgtgccacatgtgggcgaaaccccaattgcataccgcaatgttcttcttcgtaagaacggtaataagggagccggtggtcatagctatggcatcgatctaaagtcttatgacttaggtgacgagcttggcactgatcccattgaagattatgaacaaaactggaacactaagcatggcagtggtgcactccgtgaactcactcgtgagctcaatggaggtgcagtcactcgctatgtcgacaacaatttctgtggcccagatgggtaccctcttgattgcatcaaagattttctcgcacgcgcgggcaagtcaatgtgcactctttccgaacaacttgattacatcgagtcgaagagaggtgtctactgctgccgtgaccatgagcatgaaattgcctggttcactgagcgctctgataagagctacgagcaccagacacccttcgaaattaagagtgccaagaaatttgacactttcaaaggggaatgcccaaagtttgtgtttcctcttaactcaaaagtcaaagtcattcaaccacgtgttgaaaagaaaaagactgagggtttcatggggcgtatacgctctgtgtaccctgttgcatctccacaggagtgtaacaatatgcacttgtctaccttgatgaaatgtaatcattgcgatgaagtttcatggcagacgtgcgactttctgaaagccacttgtgaacattgtggcactgaaaatttagttattgaaggacctactacatgtgggtacctacctactaatgctgtagtgaaaatgccatgtcctgcctgtcaagacccagagattggacctgagcatagtgttgcagattatcacaaccactcaaacattgaaactcgactccgcaagggaggtaggactagatgttttggaggctgtgtgtttgcctatgttggctgctataataagcgtgcctactgggttcctcgtgctagtgctgatattggctcaggccatactggcattactggtgacaatgtggagaccttgaatgaggatctccttgagatactgagtcgtgaacgtgttaacattaacattgttggcgattttcatttgaatgaagaggttgccatcattttggcatctttctctgcttctacaagtgcctttattgacactataaagagtcttgattacaagtctttcaaaaccattgttgagtcctgcggtaactataaagttaccaagggaaagcccgtaaaaggtgcttggaacattggacaacagagatcagttttaacaccactgtgtggttttccctcacaggctgctggtgttatcagatcaatttttgcgcgcacacttgatgcagcaaaccactcaattcctgatttgcaaagagcagctgtcaccatacttgatggtatttctgaacagtcattacgtcttgtcgacgccatggtttatacttcagacctgctcaccaacagtgtcattattatggcatatgtaactggtggtcttgtacaacagacttctcagtggttgtctaatcttttgggcactactgttgaaaaactcaggcctatctttgaatggattgaggcgaaacttagtgcaggagttgaatttctcaaggatgcttgggagattctcaaatttctcattacaggtgtttttgacatcgtcaagggtcaaatacaggttgcttcagataacatcaaggattgtgtaaaatgcttcattgatgttgttaacaaggcactcgaaatgtgcattgatcaagtcactatcgctggcgcaaagttgcgatcactcaacttaggtgaagtcttcatcgctcaaagcaagggactttaccgtcagtgtatacgtggcaaggagcagctgcaactactcatgcctcttaaggcaccaaaagaagtaacctttcttgaaggtgattcacatgacacagtacttacctctgaggaggttgttctcaagaacggtgaactcgaagcactcgagacgcccgttgatagcttcacaaatggagctatcgttggcacaccagtctgtgtaaatggcctcatgctcttagagattaaggacaaagaacaatactgcgcattgtctcctggtttactggctacaaacaatgtctttcgcttaaaagggggtgcaccaattaaaggtgtaacctttggagaagatactgtttgggaagttcaaggttacaagaatgtgagaatcacatttgagcttgatgaacgtgttgacaaagtgcttaatgaaaagtgctctgtctacactgttgaatccggtaccgaagttactgagtttgcatgtgttgtagcagaggctgttgtgaagactttacaaccagtttctgatctccttaccaacatgggtattgatcttgatgagtggagtgtagctacattctacttatttgatgatgctggtgaagaaaacttttcatcacgtatgtattgttccttttaccctccagatgaggaagaagaggacgatgcagagtgtgaggaagaagaaattgatgaaacctgtgaacatgagtacggtacagaggatgattatcaaggtctccctctggaatttggtgcctcagctgaaacagttcgagttgaggaagaagaagaggaagactggctggatgatactactgagcaatcagagattgagccagaaccagaacctacacctgaagaaccagttaatcagtttactggttatttaaaacttactgacaatgttgccattaaatgtgttgacatcgttaaggaggcacaaagtgctaatcctatggtgattgtaaatgctgctaacatacacctgaaacatggtggtggtgtagcaggtgcactcaacaaggcaaccaatggtgccatgcaaaaggagagtgatgattacattaagctaaatggccctcttacagtaggagggtcttgtttgctttctggacataatcttgctaagaagtgtctgcatgttgttggacctaacctaaatgcaggtgaggacatccagcttcttaaggcagcatatgaaaatttcaattcacaggacatcttacttgcaccattgttgtcagcaggcatatttggtgctaaaccacttcagtctttacaagtgtgcgtgcagacggttcgtacacaggtttatattgcagtcaatgacaaagctctttatgagcaggttgtcatggattatcttgataacctgaagcctagagtggaagcacctaaacaagaggagccaccaaacacagaagattccaaaactgaggagaaatctgtcgtacagaagcctgtcgatgtgaagccaaaaattaaggcctgcattgatgaggttaccacaacactggaagaaactaagtttcttaccaataagttactcttgtttgctgatatcaatggtaagctttaccatgattctcagaacatgcttagaggtgaagatatgtctttccttgagaaggatgcaccttacatggtaggtgatgttatcactagtggtgatatcacttgtgttgtaataccctccaaaaaggctggtggcactactgagatgctctcaagagctttgaagaaagtgccagttgatgagtatataaccacgtaccctggacaaggatgtgctggttatacacttgaggaagctaagactgctcttaagaaatgcaaatctgcattttatgtactaccttcagaagcacctaatgctaaggaagagattctaggaactgtatcctggaatttgagagaaatgcttgctcatgctgaagagacaagaaaattaatgcctatatgcatggatgttagagccataatggcaaccatccaacgtaagtataaaggaattaaaattcaagagggcatcgttgactatggtgtccgattcttcttttatactagtaaagagcctgtagcttctattattacgaagctgaactctctaaatgagccgcttgtcacaatgccaattggttatgtgacacatggttttaatcttgaagaggctgcgcgctgtatgcgttctcttaaagctcctgccgtagtgtcagtatcatcaccagatgctgttactacatataatggatacctcacttcgtcatcaaagacatctgaggagcactttgtagaaacagtttctttggctggctcttacagagattggtcctattcaggacagcgtacagagttaggtgttgaatttcttaagcgtggtgacaaaattgtgtaccacactctggagagccccgtcgagtttcatcttgacggtgaggttctttcacttgacaaactaaagagtctcttatccctgcgggaggttaagactataaaagtgttcacaactgtggacaacactaatctccacacacagcttgtggatatgtctatgacatatggacagcagtttggtccaacatacttggatggtgctgatgttacaaaaattaaacctcatgtaaatcatgagggtaagactttctttgtactacctagtgatgacacactacgtagtgaagctttcgagtactaccatactcttgatgagagttttcttggtaggtacatgtctgctttaaaccacacaaagaaatggaaatttcctcaagttggtggtttaacttcaattaaatgggctgataacaattgttatttgtctagtgttttattagcacttcaacagcttgaagtcaaattcaatgcaccagcacttcaagaggcttattatagagcccgtgctggtgatgctgctaacttttgtgcactcatactcgcttacagtaataaaactgttggcgagcttggtgatgtcagagaaactatgacccatcttctacagcatgctaatttggaatctgcaaagcgagttcttaatgtggtgtgtaaacattgtggtcagaaaactactaccttaacgggtgtagaagctgtgatgtatatgggtactctatcttatgataatcttaagacaggtgtttccattccatgtgtgtgtggtcgtgatgctacacaatatctagtacaacaagagtcttcttttgttatgatgtctgcaccacctgctgagtataaattacagcaaggtacattcttatgtgcgaatgagtacactggtaactatcagtgtggtcattacactcatataactgctaaggagaccctctatcgtattgacggagctcaccttacaaagatgtcagagtacaaaggaccagtgactgatgttttctacaaggaaacatcttacactacaaccatcaagcctgtgtcgtataaactcgatggagttacttacacagagattgaaccaaaattggatgggtattataaaaaggataatgcttactatacagagcagcctatagaccttgtaccaactcaaccattaccaaatgcgagttttgataatttcaaactcacatgttctaacacaaaatttgctgatgatttaaatcaaatgacaggcttcacaaagccagcttcacgagagctatctgtcacattcttcccagacttgaatggcgatgtagtggctattgactatagacactattcagcgagtttcaagaaaggtgctaaattactgcataagccaattgtttggcacattaaccaggctacaaccaagacaacgttcaaaccaaacacttggtgtttacgttgtctttggagtacaaagccagtagatacttcaaattcatttgaagttctggcagtagaagacacacaaggaatggacaatcttgcttgtgaaagtcaacaacccacctctgaagaagtagtggaaaatcctaccatacagaaggaagtcatagagtgtgacgtgaaaactaccgaagttgtaggcaatgtcatacttaaaccatcagatgaaggtgttaaagtaacacaagagttaggtcatgaggatcttatggctgcttatgtggaaaacacaagcattaccattaagaaacctaatgagctttcactagccttaggtttaaaaacaattgccactcatggtattgctgcaattaatagtgttccttggagtaaaattttggcttatgtcaaaccattcttaggacaagcagcaattacaacatcaaattgcgctaagagattagcacaacgtgtgtttaacaattatatgccttatgtgtttacattattgttccaattgtgtacttttactaaaagtaccaattctagaattagagcttcactacctacaactattgctaaaaatagtgttaagagtgttgctaaattatgtttggatgccggcattaattatgtgaagtcacccaaattttctaaattgttcacaatcgctatgtggctattgttgttaagtatttgcttaggttctctaatctgtgtaactgctgcttttggtgtactcttatctaattttggtgctccttcttattgtaatggcgttagagaattgtatcttaattcgtctaacgttactactatggatttctgtgaaggttcttttccttgcagcatttgtttaagtggattagactcccttgattcttatccagctcttgaaaccattcaggtgacgatttcatcgtacaagctagacttgacaattttaggtctggccgctgagtgggttttggcatatatgttgttcacaaaattcttttatttattaggtctttcagctataatgcaggtgttctttggctattttgctagtcatttcatcagcaattcttggctcatgtggtttatcattagtattgtacaaatggcacccgtttctgcaatggttaggatgtacatcttctttgcttctttctactacatatggaagagctatgttcatatcatggatggttgcacctcttcgacttgcatgatgtgctataagcgcaatcgtgccacacgcgttgagtgtacaactattgttaatggcatgaagagatctttctatgtctatgcaaatggaggccgtggcttctgcaagactcacaattggaattgtctcaattgtgacacattttgcactggtagtacattcattagtgatgaagttgctcgtgatttgtcactccagtttaaaagaccaatcaaccctactgaccagtcatcgtatattgttgatagtgttgctgtgaaaaatggcgcgcttcacctctactttgacaaggctggtcaaaagacctatgagagacatccgctctcccattttgtcaatttagacaatttgagagctaacaacactaaaggttcactgcctattaatgtcatagtttttgatggcaagtccaaatgcgacgagtctgcttctaagtctgcttctgtgtactacagtcagctgatgtgccaacctattctgttgcttgaccaagctcttgtatcagacgttggagatagtactgaagtttccgttaagatgtttgatgcttatgtcgacaccttttcagcaacttttagtgttcctatggaaaaacttaaggcacttgttgctacagctcacagcgagttagcaaagggtgtagctttagatggtgtcctttctacattcgtgtcagctgcccgacaaggtgttgttgataccgatgttgacacaaaggatgttattgaatgtctcaaactttcacatcactctgacttagaagtgacaggtgacagttgtaacaatttcatgctcacctataataaggttgaaaacatgacgcccagagatcttggcgcatgtattgactgtaatgcaaggcatatcaatgcccaagtagcaaaaagtcacaatgtttcactcatctggaatgtaaaagactacatgtctttatctgaacagctgcgtaaacaaattcgtagtgctgccaagaagaacaacataccttttagactaacttgtgctacaactagacaggttgtcaatgtcataactactaaaatctcactcaagggtggtaagattgttagtacttgttttaaacttatgcttaaggccacattattgtgcgttcttgctgcattggtttgttatatcgttatgccagtacatacattgtcaatccatgatggttacacaaatgaaatcattggttacaaagccattcaggatggtgtcactcgtgacatcatttctactgatgattgttttgcaaataaacatgctggttttgacgcatggtttagccagcgtggtggttcatacaaaaatgacaaaagctgccctgtagtagctgctatcattacaagagagattggtttcatagtgcctggcttaccgggtactgtgctgagagcaatcaatggtgacttcttgcattttctacctcgtgtttttagtgctgttggcaacatttgctacacaccttccaaactcattgagtatagtgattttgctacctctgcttgcgttcttgctgctgagtgtacaatttttaaggatgctatgggcaaacctgtgccatattgttatgacactaatttgctagagggttctatttcttatagtgagcttcgtccagacactcgttatgtgcttatggatggttccatcatacagtttcctaacacttacctggagggttctgttagagtagtaacaacttttgatgctgagtactgtagacatggtacatgcgaaaggtcagaagtaggtatttgcctatctaccagtggtagatgggttcttaataatgagcattacagagctctatcaggagttttctgtggtgttgatgcgatgaatctcatagctaacatctttactcctcttgtgcaacctgtgggtgctttagatgtgtctgcttcagtagtggctggtggtattattgccatattggtgacttgtgctgcctactactttatgaaattcagacgtgtttttggtgagtacaaccatgttgttgctgctaatgcacttttgtttttgatgtctttcactatactctgtctggtaccagcttacagctttctgccgggagtctactcagtcttttacttgtacttgacattctatttcaccaatgatgtttcattcttggctcaccttcaatggtttgccatgttttctcctattgtgcctttttggataacagcaatctatgtattctgtatttctctgaagcactgccattggttctttaacaactatcttaggaaaagagtcatgtttaatggagttacatttagtaccttcgaggaggctgctttgtgtacctttttgctcaacaaggaaatgtacctaaaattgcgtagcgagacactgttgccacttacacagtataacaggtatcttgctctatataacaagtacaagtatttcagtggagccttagatactaccagctatcgtgaagcagcttgctgccacttagcaaaggctctaaatgactttagcaactcaggtgctgatgttctctaccaaccaccacagacatcaatcacttctgctgttctgcagagtggttttaggaaaatggcattcccgtcaggcaaagttgaagggtgcatggtacaagtaacctgtggaactacaactcttaatggattgtggttggatgacacagtatactgtccaagacatgtcatttgcacagcagaagacatgcttaatcctaactatgaagatctgctcattcgcaaatccaaccatagctttcttgttcaggctggcaatgttcaacttcgtgttattggccattctatgcaaaattgtctgcttaggcttaaagttgatacttctaaccctaagacacccaagtataaatttgtccgtatccaacctggtcaaacattttcagttctagcatgctacaatggttcaccatctggtgtttatcagtgtgccatgagacctaatcataccattaaaggttctttccttaatggatcatgtggtagtgttggttttaacattgattatgattgcgtgtctttctgctatatgcatcatatggagcttccaacaggagtacacgctggtactgacttagaaggtaaattctatggtccatttgttgacagacaaactgcacaggctgcaggtacagacacaaccataacattaaatgttttggcatggctgtatgctgctgttatcaatggtgataggtggtttcttaatagattcaccactactttgaatgactttaaccttgtggcaatgaagtacaactatgaacctttgacacaagatcatgttgacatattgggacctctttctgctcaaacaggaattgccgtcttagatatgtgtgctgctttgaaagagctgctgcagaatggtatgaatggtcgtactatccttggtagcactattttagaagatgagtttacaccatttgatgttgttagacaatgctctggtgttaccttccaaggtaagttcaagaaaattgttaagggcactcatcattggatgcttttaactttcttgacatcactattgattcttgttcaaagtacacagtggtcactgtttttctttgtttacgagaatgctttcttgccatttactcttggtattatggcaattgctgcatgtgctatgctgcttgttaagcataagcacgcattcttgtgcttgtttctgttaccttctcttgcaacagttgcttactttaatatggtctacatgcctgctagctgggtgatgcgtatcatgacatggcttgaattggctgacactagcttgtctggttataggcttaaggattgtgttatgtatgcttcagctttagttttgcttattctcatgacagctcgcactgtttatgatgatgctgctagacgtgtttggacactgatgaatgtcattacacttgtttacaaagtctactatggtaatgctttagatcaagctatttccatgtgggccttagttatttctgtaacctctaactattctggtgtcgttacgactatcatgtttttagctagagctatagtgtttgtgtgtgttgagtattacccattgttatttattactggcaacaccttacagtgtatcatgcttgtttattgtttcttaggctattgttgctgctgctactttggccttttctgtttactcaaccgttacttcaggcttactcttggtgtttatgactacttggtctctacacaagaatttaggtatatgaactcccaggggcttttgcctcctaagagtagtattgatgctttcaagcttaacattaagttgttgggtattggaggtaaaccatgtatcaaggttgctactgtacagtctaaaatgtctgacgtaaagtgcacatctgtggtactgctctcggttcttcaacaacttagagtagagtcatcttctaaattgtgggcacaatgtgtacaactccacaatgatattcttcttgcaaaagacacaactgaagctttcgagaagatggtttctcttttgtctgttttgctatccatgcagggtgctgtagacattaataggttgtgcgaggaaatgctcgataaccgtgctactcttcaggctattgcttcagaatttagttctttaccatcatatgccgcttatgccactgcccaggaggcctatgagcaggctgtagctaatggtgattctgaagtcgttctcaaaaagttaaagaaatctttgaatgtggctaaatctgagtttgaccgtgatgctgccatgcaacgcaagttggaaaagatggcagatcaggctatgacccaaatgtacaaacaggcaagatctgaggacaagagggcaaaagtaactagtgctatgcaaacaatgctcttcactatgcttaggaagcttgataatgatgcacttaacaacattatcaacaatgcgcgtgatggttgtgttccactcaacatcataccattgactacagcagccaaactcatggttgttgtccctgattatggtacctacaagaacacttgtgatggtaacacctttacatatgcatctgcactctgggaaatccagcaagttgttgatgcggatagcaagattgttcaacttagtgaaattaacatggacaattcaccaaatttggcttggcctcttattgttacagctctaagagccaactcagctgttaaactacagaataatgaactgagtccagtagcactacgacagatgtcctgtgcggctggtaccacacaaacagcttgtactgatgacaatgcacttgcctactataacaattcgaagggaggtaggtttgtgctggcattactatcagaccaccaagatctcaaatgggctagattccctaagagtgatggtacaggtacaatttacacagaactggaaccaccttgtaggtttgttacagacacaccaaaagggcctaaagtgaaatacttgtacttcatcaaaggcttaaacaacctaaatagaggtatggtgctgggcagtttagctgctacagtacgtcttcaggctggaaatgctacagaagtacctgccaattcaactgtgctttccttctgtgcttttgcagtagaccctgctaaagcatataaggattacctagcaagtggaggacaaccaatcaccaactgtgtgaagatgttgtgtacacacactggtacaggacaggcaattactgtaacaccagaagctaacatggaccaagagtcctttggtggtgcttcatgttgtctgtattgtagatgccacattgaccatccaaatcctaaaggattctgtgacttgaaaggtaagtacgtccaaatacctaccacttgtgctaatgacccagtgggttttacacttagaaacacagtctgtaccgtctgcggaatgtggaaaggttatggctgtagttgtgaccaactccgcgaacccttgatgcagtctgcggatgcatcaacgtttttaaacgggtttgcggtgtaagtgcagcccgtcttacaccgtgcggcacaggcactagtactgatgtcgtctacagggcttttgatatttacaacgaaaaagttgctggttttgcaaagttcctaaaaactaattgctgtcgcttccaggagaaggatgaggaaggcaatttattagactcttactttgtagttaagaggcatactatgtctaactaccaacatgaagagactatttataacttggttaaagattgtccagcggttgctgtccatgactttttcaagtttagagtagatggtgacatggtaccacatatatcacgtcagcgtctaactaaatacacaatggctgatttagtctatgctctacgtcattttgatgagggtaattgtgatacattaaaagaaatactcgtcacatacaattgctgtgatgatgattatttcaataagaaggattggtatgacttcgtagagaatcctgacatcttacgcgtatatgctaacttaggtgagcgtgtacgccaatcattattaaagactgtacaattctgcgatgctatgcgtgatgcaggcattgtaggcgtactgacattagataatcaggatcttaatgggaactggtacgatttcggtgatttcgtacaagtagcaccaggctgcggagttcctattgtggattcatattactcattgctgatgcccatcctcactttgactagggcattggctgctgagtcccatatggatgctgatctcgcaaaaccacttattaagtgggatttgctgaaatatgattttacggaagagagactttgtctcttcgaccgttattttaaatattgggaccagacataccatcccaattgtattaactgtttggatgataggtgtatccttcattgtgcaaactttaatgtgttattttctactgtgtttccacctacaagttttggaccactagtaagaaaaatatttgtagatggtgttccttttgttgtttcaactggataccattttcgtgagttaggagtcgtacataatcaggatgtaaacttacatagctcgcgtctcagtttcaaggaacttttagtgtatgctgctgatccagctatgcatgcagcttctggcaatttattgctagataaacgcactacatgcttttcagtagctgcactaacaaacaatgttgcttttcaaactgtcaaacccggtaattttaataaagacttttatgactttgctgtgtctaaaggtttctttaaggaaggaagttctgttgaactaaaacacttcttctttgctcaggatggcaacgctgctatcagtgattatgactattatcgttataatctgccaacaatgtgtgatatcagacaactcctattcgtagttgaagttgttgataaatactttgattgttacgatggtggctgtattaatgccaaccaagtaatcgttaacaatctggataaatcagctggtttcccatttaataaatggggtaaggctagactttattatgactcaatgagttatgaggatcaagatgcacttttcgcgtatactaagcgtaatgtcatccctactataactcaaatgaatcttaagtatgccattagtgcaaagaatagagctcgcaccgtagctggtgtctctatctgtagtactatgacaaatagacagtttcatcagaaattattgaagtcaatagccgccactagaggagctactgtggtaattggaacaagcaagttttacggtggctggcataatatgttaaaaactgtttacagtgatgtagaaactccacaccttatgggttgggattatccaaaatgtgacagagccatgcctaacatgcttaggataatggcctctcttgttcttgctcgcaaacataacacttgctgtaacttatcacaccgtttctacaggttagctaacgagtgtgcgcaagtattaagtgagatggtcatgtgtggcggctcactatatgttaaaccaggtggaacatcatccggtgatgctacaactgcttatgctaatagtgtctttaacatttgtcaagctgttacagccaatgtaaatgcacttctttcaactgatggtaataagatagctgacaagtatgtccgcaatctacaacacaggctctatgagtgtctctatagaaatagggatgttgatcatgaattcgtggatgagttttacgcttacctgcgtaaacatttctccatgatgattctttctgatgatgccgttgtgtgctataacagtaactatgcggctcaaggtttagtagctagcattaagaactttaaggcagttctttattatcaaaataatgtgttcatgtctgaggcaaaatgttggactgagactgaccttactaaaggacctcacgaattttgctcacagcatacaatgctagttaaacaaggagatgattacgtgtacctgccttacccagatccatcaagaatattaggcgcaggctgttttgtcgatgatattgtcaaaacagatggtacacttatgattgaaaggttcgtgtcactggctattgatgcttacccacttacaaaacatcctaatcaggagtatgctgatgtctttcacttgtatttacaatacattagaaagttacatgatgagcttactggccacatgttggacatgtattccgtaatgctaactaatgataacacctcacggtactgggaacctgagttttatgaggctatgtacacaccacatacagtcttgcaggctgtaggtgcttgtgtattgtgcaattcacagacttcacttcgttgcggtgcctgtattaggagaccattcctatgttgcaagtgctgctatgaccatgtcatttcaacatcacacaaattagtgttgtctgttaatccctatgtttgcaatgccccaggttgtgatgtcactgatgtgacacaactgtatctaggaggtatgagctattattgcaagtcacataagcctcccattagttttccattatgtgctaatggtcaggtttttggtttatacaaaaacacatgtgtaggcagtgacaatgtcactgacttcaatgcgatagcaacatgtgattggactaatgctggcgattacatacttgccaacacttgtactgagagactcaagcttttcgcagcagaaacgctcaaagccactgaggaaacatttaagctgtcatatggtattgccactgtacgcgaagtactctctgacagagaattgcatctttcatgggaggttggaaaacctagaccaccattgaacagaaactatgtctttactggttaccgtgtaactaaaaatagtaaagtacagattggagagtacacctttgaaaaaggtgactatggtgatgctgttgtgtacagaggtactacgacatacaagttgaatgttggtgattactttgtgttgacatctcacactgtaatgccacttagtgcacctactctagtgccacaagagcactatgtgagaattactggcttgtacccaacactcaacatctcagatgagttttctagcaatgttgcaaattatcaaaaggtcggcatgcaaaagtactctacactccaaggaccacctggtactggtaagagtcattttgccatcggacttgctctctattacccatctgctcgcatagtgtatacggcatgctctcatgcagctgttgatgccctatgtgaaaaggcattaaaatatttgcccatagataaatgtagtagaatcatacctgcgcgtgcgcgcgtagagtgttttgataaattcaaagtgaattcaacactagaacagtatgttttctgcactgtaaatgcattgccagaaacaactgctgacattgtagtctttgatgaaatctctatggctactaattatgacttgagtgttgtcaatgctagacttcgtgcaaaacactacgtctatattggcgatcctgctcaattaccagccccccgcacattgctgactaaaggcacactagaaccagaatattttaattcagtgtgcagacttatgaaaacaataggtccagacatgttccttggaacttgtcgccgttgtcctgctgaaattgttgacactgtgagtgctttagtttatgacaataagctaaaagcacacaaggataagtcagctcaatgcttcaaaatgttctacaaaggtgttattacacatgatgtttcatctgcaatcaacagacctcaaataggcgttgtaagagaatttcttacacgcaatcctgcttggagaaaagctgtttttatctcaccttataattcacagaacgctgtagcttcaaaaatcttaggattgcctacgcagactgttgattcatcacagggttctgaatatgactatgtcatattcacacaaactactgaaacagcacactcttgtaatgtcaaccgcttcaatgtggctatcacaagggcaaaaattggcattttgtgcataatgtctgatagagatctttatgacaaactgcaatttacaagtctagaaataccacgtcgcaatgtggctacattacaagcagaaaatgtaactggactttttaaggactgtagtaagatcattactggtcttcatcctacacaggcacctacacacctcagcgttgatataaagttcaagactgaaggattatgtgttgacataccaggcataccaaaggacatgacctaccgtagactcatctctatgatgggtttcaaaatgaattaccaagtcaatggttaccctaatatgtttatcacccgcgaagaagctattcgtcacgttcgtgcgtggattggctttgatgtagagggctgtcatgcaactagagatgctgtgggtactaacctacctctccagctaggattttctacaggtgttaacttagtagctgtaccgactggttatgttgacactgaaaataacacagaattcaccagagttaatgcaaaacctccaccaggtgaccagtttaaacatcttataccactcatgtataaaggcttgccctggaatgtagtgcgtattaagatagtacaaatgctcagtgatacactgaaaggattgtcagacagagtcgtgttcgtcctttgggcgcatggctttgagcttacatcaatgaagtactttgtcaagattggacctgaaagaacgtgttgtctgtgtgacaaacgtgcaacttgcttttctacttcatcagatacttatgcctgctggaatcattctgtgggttttgactatgtctataacccatttatgattgatgttcagcagtggggctttacgggtaaccttcagagtaaccatgaccaacattgccaggtacatggaaatgcacatgtggctagttgtgatgctatcatgactagatgtttagcagtccatgagtgctttgttaagcgcgttgattggtctgttgaataccctattataggagatgaactgagggttaattctgcttgcagaaaagtacaacacatggttgtgaagtctgcattgcttgctgataagtttccagttcttcatgacattggaaatccaaaggctatcaagtgtgtgcctcaggctgaagtagaatggaagttctacgatgctcagccatgtagtgacaaagcttacaaaatagaggaactcttctattcttatgctacacatcacgataaattcactgatggtgtttgtttgttttggaattgtaacgttgatcgttacccagccaatgcaattgtgtgtaggtttgacacaagagtcttgtcaaacttgaacttaccaggctgtgatggtggtagtttgtatgtgaataagcatgcattccacactccagctttcgataaaagtgcatttactaatttaaagcaattgcctttcttttactattctgatagtccttgtgagtctcatggcaaacaagtagtgtcggatattgattatgttccactcaaatctgctacgtgtattacacgatgcaatttaggtggtgctgtttgcagacaccatgcaaatgagtaccgacagtacttggatgcatataatatgatgatttctgctggatttagcctatggatttacaaacaatttgatacttataacctgtggaatacatttaccaggttacagagtttagaaaatgtggcttataatgttgttaataaaggacactttgatggacacgccggcgaagcacctgtttccatcattaataatgctgtttacacaaaggtagatggtattgatgtggagatctttgaaaataagacaacacttcctgttaatgttgcatttgagctttgggctaagcgtaacattaaaccagtgccagagattaagatactcaataatttgggtgttgatatcgctgctaatactgtaatctgggactacaaaagagaagccccagcacatgtatctacaataggtgtctgcacaatgactgacattgccaagaaacctactgagagtgcttgttcttcacttactgtcttgtttgatggtagagtggaaggacaggtagacctttttagaaacgcccgtaatggtgttttaataacagaaggttcagtcaaaggtctaacaccttcaaagggaccagcacaagctagcgtcaatggagtcacattaattggagaatcagtaaaaacacagtttaactactttaagaaagtagacggcattattcaacagttgcctgaaacctactttactcagagcagagacttagaggattttaagcccagatcacaaatggaaactgactttctcgagctcgctatggatgaattcatacagcgatataagctcgagggctatgccttcgaacacatcgtttatggagatttcagtcatggacaacttggcggtcttcatttaatgataggcttagccaagcgctcacaagattcaccacttaaattagaggattttatccctatggacagcacagtgaaaaattacttcataacagatgcgcaaacaggttcatcaaaatgtgtgtgttctgtgattgatcttttacttgatgactttgtcgagataataaagtcacaagatttgtcagtgatttcaaaagtggtcaaggttacaattgactatgctgaaatttcattcatgctttggtgtaaggatggacatgttgaaaccttctacccaaaactacaagcaagtcaagcgtggcaaccaggtgttgcgatgcctaacttgtacaagatgcaaagaatgcttcttgaaaagtgtgaccttcagaattatggtgaaaatgctgttataccaaaaggaataatgatgaatgtcgcaaagtatactcaactgtgtcaatacttaaatacacttactttagctgtaccctacaacatgagagttattcactttggtgctggctctgataaaggagttgcaccaggtacagctgtgctcagacaatggttgccaactggcacactacttgtcgattcagatcttaatgacttcgtctccgacgcagattctactttaattggagactgtgcaacagtacatacggctaataaatgggaccttattattagcgatatgtatgaccctaggaccaaacatgtgacaaaagagaatgactctaaagaagggtttttcacttatctgtgtggatttataaagcaaaaactagccctgggtggttctatagctgtaaagataacagagcattcttggaatgctgacctttacaagcttatgggccatttctcatggtggacagcttttgttacaaatgtaaatgcatcatcatcggaagcatttttaattggggctaactatcttggcaagccgaaggaacaaattgatggctataccatgcatgctaactacattttctggaggaacacaaatcctatccagttgtcttcctattcactctttgacatgagcaaatttcctcttaaattaagaggaactgctgtaatgtctcttaaggagaatcaaatcaatgatatgatttattctcttctggaaaaaggtaggcttatcattagagaaaacaacagagttgtggtttcaagtgatattcttgttaacaactaaacgaacatgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgcgcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataaacgaacttatggatttgtttatgagattttttactcttagatcaattactgcacagccagtaaaaattgacaatgcttctcctgcaagtactgttcatgctacagcaacgataccgctacaagcctcactccctttcggatggcttgttattggcgttgcatttcttgctgtttttcagagcgctaccaaaataattgcgctcaataaaagatggcagctagccctttataagggcttccagttcatttgcaatttactgctgctatttgttaccatctattcacatcttttgcttgtcgctgcaggtatggaggcgcaatttttgtacctctatgccttgatatattttctacaatgcatcaacgcatgtagaattattatgagatgttggctttgttggaagtgcaaatccaagaacccattactttatgatgccaactactttgtttgctggcacacacataactatgactactgtataccatataacagtgtcacagatacaattgtcgttactgaaggtgacggcatttcaacaccaaaactcaaagaagactaccaaattggtggttattctgaggataggcactcaggtgttaaagactatgtcgttgtacatggctatttcaccgaagtttactaccagcttgagtctacacaaattactacagacactggtattgaaaatgctacattcttcatctttaacaagcttgttaaagacccaccgaatgtgcaaatacacacaatcgacggctcttcaggagttgctaatccagcaatggatccaatttatgatgagccgacgacgactactagcgtgcctttgtaagcacaagaaagtgagtacgaacttatgtactcattcgtttcggaagaaacaggtacgttaatagttaatagcgtacttctttttcttgctttcgtggtattcttgctagtcacactagccatccttactgcgcttcgattgtgtgcgtactgctgcaatattgttaacgtgagtttagtaaaaccaacggtttacgtctactcgcgtgttaaaaatctgaactcttctgaaggagttcctgatcttctggtctaaacgaactaactattattattattctgtttggaactttaacattgcttatcatggcagacaacggtactattaccgttgaggagcttaaacaactcctggaacaatggaacctagtaataggtttcctattcctagcctggattatgttactacaatttgcctattctaatcggaacaggtttttgtacataataaagcttgttttcctctggctcttgtggccagtaacacttgcttgttttgtgcttgctgctgtctacagaattaattgggtgactggcgggattgcgattgcaatggcttgtattgtaggcttgatgtggcttagctacttcgttgcttccttcaggctgtttgctcgtacccgctcaatgtggtcattcaacccagaaacaaacattcttctcaatgtgcctctccgggggacaattgtgaccagaccgctcatggaaagtgaacttgtcattggtgctgtgatcattcgtggtcacttgcgaatggccggacactccctagggcgctgtgacattaaggacctgccaaaagagatcactgtggctacatcacgaacgctttcttattacaaattaggagcgtcgcagcgtgtaggcactgattcaggttttgctgcatacaaccgctaccgtattggaaactataaattaaatacagaccacgccggtagcaacgacaatattgctttgctagtacagtaagtgacaacagatgtttcatcttgttgacttccaggttacaatagcagagatattgattatcattatgaggactttcaggattgctatttggaatcttgacgttataataagttcaatagtgagacaattatttaagcctctaactaagaagaattattcggagttagatgatgaagaacctatggagttagattatccataaaacgaacatgaaaattattctcttcctgacattgattgtatttacatcttgcgagctatatcactatcaggagtgtgttagaggtacgactgtactactaaaagaaccttgcccatcaggaacatacgagggcaattcaccatttcaccctcttgctgacaataaatttgcactaacttgcactagcacacactttgcttttgcttgtgctgacggtactcgacatacctatcagctgcgtgcaagatcagtttcaccaaaacttttcatcagacaagaggaggttcaacaagagctctactcgccactttttctcattgttgctgctctagtatttttaatactttgcttcaccattaagagaaagacagaatgaatgagctcactttaattgacttctatttgtgctttttagcctttctgctattccttgttttaataatgcttattatattttggttttcactcgaaatccaggatctagaagaaccttgtaccaaagtctaaacgaacatgaaacttctcattgttttgacttgtatttctctatgcagttgcatatgcactgtagtacagcgctgtgcatctaataaacctcatgtgcttgaagatccttgtaaggtacaacactaggggtaatacttatagcactgcttggctttgtgctctaggaaaggttttaccttttcatagatggcacactatggttcaaacatgcacacctaatgttactatcaactgtcaagatccagctggtggtgcgcttatagctaggtgttggtaccttcatgaaggtcaccaaactgctgcatttagagacgtacttgttgttttaaataaacgaacaaattaaaatgtctgataatggaccccaatcaaaccaacgtagtgccccccgcattacatttggtggacccacagattcaactgacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgacatggatgatttctccagacaacttcaaaattccatgagtggagcttctgctgattcaactcaggcataaacactcatgatgaccacacaaggcagatgggctatgtaaacgttttcgcaattccgtttacgatacatagtctactcttgtgcagaatgaattctcgtaactaaacagcacaagtaggtttagttaactttaatctcacatagcaatctttaatcaatgtgtaacattagggaggacttgaaagagccaccacattttcatcgaggccacgcggagtacgatcgagggtacagtgaataatgctagggagagctgcctatatggaagagccctaatgtgtaaaattaattttagtagtgctatccccatgtgattttaatagcttcttaggagaatgacaaaaaaaaaaaaaaaaaaaaaaaa

SEQ ID NO: 4 - DNA, 30119 bp, Middle East respiratory syndrome-related coronavirus
  source 1..30119: mol_type = genomic DNA
gatttaagtgaatagcttggctatctcacttcccctcgttctcttgcagaactttgattttaacgaacttaaataaaagccctgttgtttagcgtatcgttgcacttgtctggtgggattgtggcattaatttgcctgctcatctaggcagtggacatatgctcaacactgggtataattctaattgaatactatttttcagttagagcgtcgtgtctcttgtacgtctcggtcacaatacacggtttcgtccggtgcgtggcaattcggggcacatcatgtctttcgtggctggtgtgaccgcgcaaggtgcgcgcggtacgtatcgagcagcgctcaactctgaaaaacatcaagaccatgtgtctctaactgtgccactctgtggttcaggaaacctggttgaaaaactttcaccatggttcatggatggcgaaaatgcctatgaagtggtgaaggccatgttacttaaaaaggagccacttctctatgtgcccatccggctggctggacacactagacacctcccaggtcctcgtgtgtacctggttgagaggctcattgcttgtgaaaatccattcatggttaaccaattggcttatagctctagtgcaaatggcagcctggttggcacaactttgcagggcaagcctattggtatgttcttcccttatgacatcgaacttgtcacaggaaagcaaaatattctcctgcgcaagtatggccgtggtggttatcactacaccccattccactatgagcgagacaacacctcttgccctgagtggatggacgattttgaggcggatcctaaaggcaaatatgcccagaatctgcttaagaagttgattggcggtgatgtcactccagttgaccaatacatgtgtggcgttgatggaaaacccattagtgcctacgcatttttaatggccaaggatggaataaccaaactggctgatgttgaagcggacgtcgcagcacgtgctgatgacgaaggcttcatcacattaaagaacaatctatatagattggtttggcatgttgagcgtaaagacgttccatatcctaagcaatctatttttactattaatagtgtggtccaaaaggatggtgttgaaaacactcctcctcactattttactcttggatgcaaaattttaacgctcaccccacgcaacaagtggagtggcgtttctgacttgtccctcaaacaaaaactcctttacaccttctatggtaaggagtcacttgagaacccaacctacatttaccactccgcattcattgagtgtggaagttgtggtaatgattcctggcttacagggaatgctatccaagggtttgcctgtggatgtggggcatcatatacagctaatgatgtcgaagtccaatcatctggcatgattaagccaaatgctcttctttgtgctacttgcccctttgctaagggtgatagctgttcttctaattgcaaacattcagttgctcagttggttagttacctttctgaacgctgtaatgttattgctgattctaagtccttcacacttatctttggtggcgtagcttacgcctactttggatgtgaggaaggtactatgtactttgtgcctagagctaagtctgttgtctcaaggattggagactccatctttacaggctgtactggctcttggaacaaggtcactcaaattgctaacatgttcttggaacagactcagcattcccttaactttgtgggagagttcgttgtcaacgatgttgtcctcgcaattctctctggaaccacaactaatgttgacaaaatacgccagcttctcaaaggtgtcacccttgacaagttgcgtgattatttagctgactatgacgtagcagtcactgccggcccattcatggataatgctattaatgttggtggtacaggattacagtatgccgccattactgcaccttatgtagttctcactggcttaggtgagtcctttaagaaagttgcaaccataccgtataaggtttgcaactctgttaaggatactctggcttattatgctcacagcgtgttgtacagagtttttccttatgacatggattctggtgtgtcatcctttagtgaactactttttgattgcgttgatctttcagtagcttctacctattttttagtccgcatcttgcaagataagactggcgactttatgtctacaattattacttcctgccaaactgctgttagtaagcttctagatacatgttttgaagctacagaagcaacatttaacttcttgttagatttggcaggattgttcagaatctttctccgcaatgcctatgtgtacacttcacaagggtttgtggtggtcaatggcaaagtttctacacttgtcaaacaagtgttagacttgcttaataagggtatgcaacttttgcatacaaaggtctcctgggctggttctaaaatcattgctgttatctacagcggcagggagtctctaatattcccatcgggaacctattactgtgtcaccactaaggctaagtccgttcaacaagatcttgacgttattttgcctggtgagttttccaagaagcagttaggactgctccaacctactgacaattctacaactgttagtgttactgtatccagtaacatggttgaaactgttgtgggtcaacttgagcaaactaatatgcatagtcctgatgttatagtaggtgactatgtcattattagtgaaaaattgtttgtgcgtagtaaggaagaagacggatttgccttctaccctgcttgcactaatggtcatgctgtaccgactctctttagacttaagggaggtgcacctgtaaaaaaagtagcctttggcggtgatcaagtacatgaggttgctgctgtaagaagtgttactgtcgagtacaacattcatgctgtattagacacactacttgcttcttctagtcttagaacctttgttgtagataagtctttgtcaattgaggagtttgctgacgtagtaaaggaacaagtctcagacttgcttgttaaattactgcgtggaatgccgattccagattttgatttagacgattttattgacgcaccatgctattgctttaacgctgagggtgatgcatcctggtcttctactatgatcttctctcttcaccccgtcgagtgtgacgaggagtgttctgaagtagaggcttcagatttagaagaaggtgaatcagagtgcatttctgagacttcaactgaacaagttgacgtttctcatgagacttctgacgacgagtgggctgctgcagttgatgaagcgttccctctcgatgaagcagaagatgttactgaatctgtgcaagaagaagcacaaccagtagaagtacctgttgaagatattgcgcaggttgtcatagctgacaccttacaggaaactcctgttgtgcctgatactgttgaagtcccaccgcaagtggtgaaacttccgtctgcacctcagactatccagcccgaggtaaaagaagttgcacctgtctatgaggctgataccgaacagacacagaatgttactgttaaacctaagaggttacgcaaaaagcgtaatgttgaccctttgtccaattttgaacataaggttattacagagtgcgttaccatagttttaggtgacgcaattcaagtagccaagtgctatggggagtctgtgttagttaatgctgctaacacacatcttaagcatggcggtggtatcgctggtgctattaatgcggcttcaaaaggggctgtccaaaaagagtcagatgagtatattctggctaaagggccgttacaagtaggagattcagttctcttgcaaggccattctctagctaagaatatcctgcatgtcgtaggcccagatgcccgcgctaaacaggatgtttctctccttagtaagtgctataaggctatgaatgcatatcctcttgtagtcactcctcttgtttcagcaggcatatttggtgtaaaaccagctgtgtcttttgattatcttattagggaggctaagactagagttttagtcgtcgttaattcccaagatgtctataagagtcttaccatagttgacattccacagagtttgactttttcatatgatgggttacgtggcgcaatacgtaaagctaaagattatggttttactgtttttgtgtgcacagacaactctgctaacactaaagttcttaggaacaagggtgttgattatactaagaagtttcttacagttgacggtgtgcaatattattgctacacgtctaaggacactttagatgatatcttacaacaggctaataagtctgttggtattatatctatgcctttgggatatgtgtctcatggtttagacttaatgcaagcagggagtgtcgtgcgtagagttaacgtgccctacgtgtgtctcctagctaataaagagcaagaagctattttgatgtctgaagacgttaagttaaacccttcagaagattttataaagcacgtccgcactaatggtggttacaattcttggcatttagtcgagggtgaactattggtgcaagacttacgcttaaataagctcctgcattggtctgatcaaaccatatgctacaaggatagtgtgttttatgttgtaaagaatagtacagcttttccatttgaaacactttcagcatgtcgtgcgtatttggattcacgcacgacacagcagttaacaatcgaagtcttagtgactgtcgatggtgtaaattttagaacagtcgttctaaataataagaacacttatagatcacagcttggatgcgttttctttaatggtgctgatatttctgacaccattcctgatgagaaacagaatggtcacagtttatatctagcagacaatttgactgctgatgaaacaaaggcgcttaaagagttatatggccccgttgatcctactttcttacacagattctattcacttaaggctgcagtccatgggtggaagatggttgtgtgtgataaggtacgttctctcaaattgagtgataataattgttatcttaatgcagttattatgacacttgatttattgaaggacattaaatttgttatacctgctctacagcatgcatttatgaaacataagggcggtgattcaactgacttcatagccctcattatggcttatggcaattgcacatttggtgctccagatgatgcctctcggttacttcataccgtgcttgcaaaggctgagttatgctgttctgcacgcatggtttggagagagtggtgcaatgtctgtggcataaaagatgttgttctacaaggcttaaaagcttgttgttacgtgggtgtgcaaactgttgaagatctgcgtgctcgcatgacatatgtatgccagtgtggtggtgaacgtcatcggcaattagtcgaacacaccaccccctggttgctgctctcaggcacaccaaatgaaaaattggtgacaacctccacggcgcctgattttgtagcatttaatgtctttcagggcattgaaacggctgttggccattatgttcatgctcgcctgaagggtggtcttattttaaagtttgactctggcaccgttagcaagacttcagactggaagtgcaaggtgacagatgtacttttccccggccaaaaatacagtagcgattgtaatgtcgtacggtattctttggacggtaatttcagaacagaggttgatcccgacctatctgctttctatgttaaggatggtaaatactttacaagtgaaccacccgtaacatattcaccagctacaattttagctggtagtgtctacactaatagctgccttgtatcgtctgatggacaacctggcggtgatgctattagtttgagttttaataaccttttagggtttgattctagtaaaccagtcactaagaaatacacttactccttcttgcctaaagaagacggcgatgtgttgttggctgagtttgacacttatgaccctatttataagaatggtgccatgtataaaggcaaaccaattctttgggtcaataaagcatcttatgatactaatcttaataagttcaatagagctagtttgcgtcaaatttttgacgtagcccccattgaactcgaaaataaattcacacctttgagtgtggagtctacaccagttgaacctccaactgtagatgtggtagcacttcaacaggaaatgacaattgtcaaatgtaagggtttaaataaacctttcgtgaaggacaatgtcagtttcgttgctgatgattcaggtactcccgttgttgagtatctgtctaaagaagacctacatacattgtatgtagaccctaagtatcaagtcattgtcttaaaagacaatgtactttcttctatgcttagattgcacaccgttgagtcaggtgatattaacgttgttgcagcttccggatctttgacacgtaaagtgaagttactatttagggcttcattttatttcaaagaatttgctacccgcactttcactgctaccactgctgtaggtagttgtataaagagtgtagtgcggcatctaggtgttactaaaggcatattgacaggctgttttagttttgccaagatgttatttatgcttccactagcttactttagtgattcaaaactcggcaccacagaggttaaagtgagtgctttgaaaacagccggcgttgtgacaggtaatgttgtaaaacagtgttgcactgctgctgttgatttaagtatggataagttgcgccgtgtggattggaaatcaaccctacggttgttacttatgttatgcacaactatggtattgttgtcttctgtgtatcacttgtatgtcttcaatcaggtcttatcaagtgatgttatgtttgaagatgcccaaggtttgaaaaagttctacaaagaagttagagcttacctaggaatctcttctgcttgtgacggtcttgcttcagcttatagggcgaattcctttgatgtacctacattctgcgcaaaccgttctgcaatgtgtaattggtgcttgattagccaagattccataactcactacccagctcttaagatggttcaaacacatcttagccactatgttcttaacatagattggttgtggtttgcatttgagactggtttggcatacatgctctatacctcggccttcaactggttgttgttggcaggtacattgcattatttctttgcacagacttccatatttgtagactggcggtcatacaattatgctgtgtctagtgccttctggttattcacccacattccaatggcgggtttggtacgaatgtataatttgttagcatgcctttggcttttacgcaagttttatcagcatgtaatcaatggttgcaaagatacggcatgcttgctctgctataagaggaaccgacttactagagttgaagcttctaccgttgtctgtggtggaaaacgtacgttttatatcacagcaaatggcggtatttcattctgtcgtaggcataattggaattgtgtggattgtgacactgcaggtgtggggaataccttcatctgtgaagaagtcgcaaatgacctcactaccgccctacgcaggcctattaacgctacggatagatcacattattatgtggattccgttacagttaaagagactgttgttcagtttaattatcgtagagacggtcaaccattctacgagcggtttcccctctgcgcttttacaaatctagataagttgaagttcaaagaggtctgtaaaactactactggtatacctgaatacaactttatcatctacgactcatcagatcgtggccaggaaagtttagctaggtctgcatgtgtttattattctcaagtcttgtgtaaatcaattcttttggttgactcaagtttggttacttctgttggtgattctagtgaaatcgccactaaaatgtttgattcctttgttaatagtttcgtctcgctgtataatgtcacacgcgataagttggaaaaacttatctctactgctcgtgatggcgtaaggcgaggcgataacttccatagtgtcttaacaacattcattgacgcagcacgaggccccgcaggtgtggagtctgatgttgagaccaatgaaattgttgactctgtgcagtatgctcataaacatgacatacaaattactaatgagagctacaataattatgtaccctcatatgttaaacctgatagtgtgtctaccagcgatttaggtagtctcattgattgtaatgcggcttcagttaaccaaattgtcttgcgtaattctaatggtgcttgcatttggaacgctgctgcatatatgaaactctcggatgcacttaaacgacagattcgcattgcatgccgtaagtgtaatttagctttccggttaaccacctcaaagctacgcgctaatgataatatcttatcagttagattcactgctaacaaaattgttggtggtgctcctacatggtttaatgcgttgcgtgactttacgttaaagggttatgttcttgctaccattattgtgtttctgtgtgctgtactgatgtatttgtgtttacctacattttctatggcacctgttgaattttatgaagaccgcatcttggactttaaagttcttgataatggtatcattagggatgtaaatcctgatgataagtgctttgctaataagcaccggtccttcacacaatggtatcatgagcatgttggtggtgtctatgacaactctatcacatgcccattgacagttgcagtaattgctggagttgctggtgctcgcattccagacgtacctactacattggcttgggtgaacaatcagataattttctttgtttctcgagtctttgctaatacaggcagtgtttgctacactcctatagatgagataccctataagagtttctctgatagtggttgcattcttccatctgagtgcactatgtttagggatgcagagggccgtatgacaccatactgccatgatcctactgttttgcctggggcttttgcgtacagtcagatgaggcctcatgttcgttacgacttgtatgatggtaacatgtttattaaatttcctgaagtagtatttgaaagtacacttaggattactagaactctgtcaactcagtactgccggttcggtagttgtgagtatgcacaagagggtgtttgtattaccacaaatggctcgtgggccatttttaatgaccaccatcttaatagacctggtgtctattgtggctctgattttattgacattgtcaggcggttagcagtatcactgttccagcctattacttatttccaattgactacctcattggtcttgggtataggtttgtgtgcgttcctgactttgctcttctattatattaataaagtaaaacgtgcttttgcagattacacccagtgtgctgtaattgctgttgttgctgctgttcttaatagcttgtgcatctgctttgttacctctataccattgtgtatagtaccttacactgcattgtactattatgctacattctattttactaatgagcctgcatttattatgcatgtttcttggtacattatgttcgggcctatcgttcccatatggatgacctgcgtctatacagttgcaatgtgctttagacacttcttctgggttttagcttattttagtaagaaacatgtagaagtttttactgatggtaagcttaattgtagtttccaggacgctgcctctaatatctttgttattaacaaggacacttatgcagctcttagaaactctttaactaatgatgcctattcacgatttttggggttgtttaacaagtataagtacttctctggtgctatggaaacagccgcttatcgtgaagctgcagcatgtcatcttgctaaagccttacaaacatacagcgagactggtagtgatcttctttaccaaccacccaactgtagcataacctctggcgtgttgcaaagcggtttggtgaaaatgtcacatcccagtggagatgttgaggcttgtatggttcaggttacctgcggtagcatgactcttaatggtctttggcttgacaacacagtctggtgcccacgacacgtaatgtgcccggctgaccagttgtctgatcctaattatgatgccttgttgatttctatgactaatcatagtttcagtgtgcaaaaacacattggcgctccagcaaacttgcgtgttgttggtcatgccatgcaaggcactcttttgaagttgactgtcgatgttgctaaccctagcactccagcctacacttttacaacagtgaaacctggcgcagcatttagtgtgttagcatgctataatggtcgtccgactggtacattcactgttgtaatgcgccctaactacacaattaagggttcctttctgtgtggttcttgtggtagtgttggttacaccaaggagggtagtgtgatcaatttctgttacatgcatcaaatggaacttgctaatggtacacataccggttcagcatttgatggtactatgtatggtgcctttatggataaacaagtgcaccaagttcagttaacagacaaatactgcagtgttaatgtagtagcttggctttacgcagcaatacttaatggttgcgcttggtttgtaaaacctaatcgcactagtgttgtttcttttaatgaatgggctcttgccaaccaattcactgaatttgttggcactcaatccgttgacatgttagctgtcaaaacaggcgttgctattgaacagctgctttatgcgatccaacaactgtatactgggttccagggaaagcaaatccttggcagtaccatgttggaagatgaattcacacctgaggatgttaatatgcagattatgggtgtggttatgcagagtggtgtgagaaaagttacatatggtactgcgcattggttgtttgcgacccttgtctcaacctatgtgataatcttacaagccactaaatttactttgtggaactacttgtttgagactattcccacacagttgttcccactcttatttgtgactatggccttcgttatgttgttggttaaacacaaacacacctttttgacacttttcttgttgcctgtggctatttgtttgacttatgcaaacatagtctacgagcccactactcccatttcgtcagcgctgattgcagttgcaaattggcttgcccccactaatgcttatatgcgcactacacatactgatattggtgtctacattagtatgtcacttgtattagtcattgtagtgaagagattgtacaacccatcactttctaactttgcgttagcattgtgcagtggtgtaatgtggttgtacacttatagcattggagaagcctcaagccccattgcctatctggtttttgtcactacactcactagtgattatacgattacagtctttgttactgtcaaccttgcaaaagtttgcacttatgccatctttgcttactcaccacagcttacacttgtgtttccggaagtgaagatgatacttttattatacacatgtttaggtttcatgtgtacttgctattttggtgtcttctctcttttgaaccttaagcttagagcacctatgggtgtctatgactttaaggtctcaacacaagagttcagattcatgactgctaacaatctaactgcacctagaaattcttgggaggctatggctctgaactttaagttaataggtattggcggtacaccttgtataaaggttgctgctatgcagtctaaacttacagatcttaaatgcacatctgtggttctcctctctgtgctccaacagttacacttagaggctaatagtagggcctgggctttctgtgttaaatgccataatgatatattggcagcaacagaccccagtgaggctttcgagaaattcgtaagtctctttgctactttaatgactttttctggtaatgtagatcttgatgcgttagctagtgatatttttgacactcctagcgtacttcaagctactctttctgagttttcacacttagctacctttgctgagttggaagctgcgcagaaagcctatcaggaagctatggactctggtgacacctcaccacaagttcttaaggctttgcagaaggctgttaatatagctaaaaacgcctatgagaaggataaggcagtggcccgtaagttagaacgtatggctgatcaggctatgacttctatgtataagcaagcacgtgctgaagacaagaaagcaaaaattgtcagtgctatgcaaactatgttgtttggtatgattaagaagctcgacaacgatgttcttaatggtatcatttctaacgctaggaatggttgtatacctcttagtgtcatcccactgtgtgcttcaaataaacttcgcgttgtaattcctgacttcaccgtctggaatcaggtagtcacatatccctcgcttaactacgctggggctttgtgggacattacagttataaacaatgtggacaatgaaattgttaagtcttcagatgttgtagacagcaatgaaaatttaacatggccacttgttttagaatgcactagggcatccacttctgccgttaagttgcaaaataatgagatcaaaccttcaggtctaaaaaccatggttgtgtctgcgggtcaagagcaaactaactgtaatactagttccttagcttattacgaacctgtgcagggtcgtaaaatgctgatggctcttctttctgataatgcctatctcaaatgggcgcgtgttgaaggtaaggacggatttgtcagtgtagagctacaacctccttgcaaattcttgattgcgggaccaaaaggacctgaaatccgatatctctattttgttaaaaatcttaacaaccttcatcgcgggcaagtgttagggcacattgctgcgactgttagattgcaagctggttctaacaccgagtttgcctctaattcctcggtgttgtcacttgttaacttcaccgttgatcctcaaaaagcttatctcgatttcgtcaatgcgggaggtgccccattgacaaattgtgttaagatgcttactcctaaaactggtacaggtatagctatatctgttaaaccagagagtacagctgatcaagagacttatggtggagcttcagtgtgtctctattgccgtgcgcatatagaacatcctgatgtctctggtgtttgtaaatataagggtaagtttgtccaaatccctgctcagtgtgtccgtgaccctgtgggattttgtttgtcaaataccccctgtaatgtctgtcaatattggattggatatgggtgcaattgtgactcgcttaggcaagcagcactgccccaatctaaagattccaattttttaaacgagtccggggttctattgtaaatgcccgaatagaaccctgttcaagtggtttgtccactgatgtcgtctttagggcatttgacatctgcaactataaggctaaggttgctggtattggaaaatactacaagactaatacttgtaggtttgtagaattagatgaccaagggcatcatttagactcctattttgtcgttaagaggcatactatggagaattatgaactagagaagcactgttacgacttgttacgtgactgtgatgctgtagctccccatgatttcttcatctttgatgtagacaaagttaaaacacctcatattgtacgtcagcgtttaactgagtacactatgatggatcttgtatatgccctgaggcactttgatcaaaatagcgaagtgcttaaggctatcttagtgaagtatggttgctgtgatgttacctactttgaaaataaactctggtttgattttgttgaaaatcccagtgttattggtgtttatcataaacttggagaacgtgtacgccaagctatcttaaacactgttaaattttgtgaccacatggtcaaggctggtttagtcggtgtgctcacactagacaaccaggaccttaatggcaagtggtatgattttggtgacttcgtaatcactcaacctggttcaggagtagctatagttgatagctactattcttatttgatgcctgtgctctcaatgaccgattgtctggccgctgagacacatagggattgtgattttaataaaccactcattgagtggccacttactgagtatgattttactgattataaggtacaactctttgagaagtactttaaatattgggatcagacgtatcacgcaaattgcgttaattgtactgatgaccgttgtgtgttacattgtgctaatttcaatgtattgtttgctatgaccatgcctaagacttgtttcggacccatagtccgaaagatctttgttgatggcgtgccatttgtagtatcttgtggttatcactacaaagaattaggtttagtcatgaatatggatgttagtctccatagacataggctctctcttaaggagttgatgatgtatgccgctgatccagccatgcacattgcctcctctaacgcttttcttgatttgaggacatcatgttttagtgtcgctgcacttacaactggtttgacttttcaaactgtgcggcctggcaattttaaccaagacttctatgatttcgtggtatctaaaggtttctttaaggagggctcttcagtgacgctcaaacattttttctttgctcaagatggtaatgctgctattacagattataattactattcttataatctgcctactatgtgtgacatcaaacaaatgttgttctgcatggaagttgtaaacaagtacttcgaaatctatgacggtggttgtcttaatgcttctgaagtggttgttaataatttagacaagagtgctggccatccttttaataagtttggcaaagctcgtgtctattatgagagcatgtcttaccaggagcaagatgaactttttgccatgacaaagcgtaacgtcattcctaccatgactcaaatgaatctaaaatatgctattagtgctaagaatagagctcgcactgttgcaggcgtgtccatacttagcacaatgactaatcgccagtaccatcagaaaatgcttaagtccatggctgcaactcgtggagcgacttgcgtcattggtactacaaagttctacggtggctgggatttcatgcttaaaacattgtacaaagatgttgataatccgcatcttatgggttgggattaccctaagtgtgatagagctatgcctaatatgtgtagaatcttcgcttcactcatattagctcgtaaacatggcacttgttgtactacaagggacagattttatcgcttggcaaatgagtgtgctcaggtgctaagcgaatatgttctatgtggtggtggttactacgtcaaacctggaggtaccagtagcggagatgccaccactgcatatgccaatagtgtctttaacattttgcaggcgacaactgctaatgtcagtgcacttatgggtgctaatggcaacaagattgttgacaaagaagttaaagacatgcagtttgatttgtatgtcaatgtttacaggagcactagcccagaccccaaatttgttgataaatactatgcttttcttaataagcacttttctatgatgatactgtctgatgacggtgtcgtttgctataatagtgattatgcagctaagggttacattgctggaatacagaattttaaggaaacgctgtattatcagaacaatgtctttatgtctgaagctaaatgctgggtggaaaccgatctgaagaaagggccacatgaattctgttcacagcatacgctttatattaaggatggcgacgatggttacttccttccttatccagacccttcaagaattttgtctgccggttgctttgtagatgatatcgttaagactgacggtacactcatggtagagcggtttgtgtctttggctatagatgcttaccctctcacaaagcatgaagatatagaataccagaatgtattctgggtctacttacagtatatagaaaaactgtataaagaccttacaggacacatgcttgacagttattctgtcatgctatgtggtgataattctgctaagttttgggaagaggcattctatagagatctctatagttcgcctaccactttgcaggctgtcggttcatgcgttgtatgccattcacagacttccctacgctgtgggacatgcatccgtagaccatttctctgctgtaaatgctgctatgatcatgttatagcaactccacataagatggttttgtctgtttctccttacgtttgtaatgcccctggttgtggcgtttcagacgttactaagctatatttaggtggtatgagctacttttgtgtagatcatagacctgtgtgtagttttccactttgcgctaatggtcttgtattcggcttatacaagaatatgtgcacaggtagtccttctatagttgaatttaataggttggctacctgtgactggactgaaagtggtgattacacccttgccaatactacaacagaaccactcaaactttttgctgctgagactttacgtgccactgaagaggcgtctaagcagtcttatgctattgccaccatcaaagaaattgttggtgagcgccaactattacttgtgtgggaggctggcaagtccaaaccaccactcaatcgtaattatgtttttactggttatcatataaccaaaaatagtaaagtgcagctcggtgagtacattttcgagcgcattgattatagtgatgctgtatcctacaagtctagtacaacgtataaactgactgtaggtgacatcttcgtacttacctctcactctgtggctaccttgacggcgcccacaattgtgaatcaagagaggtatgttaaaattactgggttgtacccaaccattacggtacctgaagagttcgcaagtcatgttgccaacttccaaaaatcaggttatagtaaatatgtcactgttcagggaccacctggcactggcaaaagtcattttgctatagggttagcgatttactaccctacagcacgtgttgtttatacagcatgttcacacgcagctgttgatgctttgtgtgaaaaagcttttaaatatttgaacattgctaaatgttcccgtatcattcctgcaaaggcacgtgttgagtgctatgacaggtttaaagttaatgagacaaattctcaatatttgtttagtactattaatgctctaccagaaacttctgccgatattctggtggttgatgaggttagtatgtgcactaattatgatctttcaattattaatgcacgtattaaagctaagcacattgtctatgtaggagatccagcacagttgccagctcctaggactttgttgactagaggcacattggaaccagaaaatttcaatagtgtcactagattgatgtgtaacttaggtcctgacatatttttaagtatgtgctacaggtgtcctaaggaaatagtaagcactgtgagcgctcttgtctacaataataaattgttagccaagaaggagctttcaggccagtgctttaaaatactctataagggcaatgtgacgcatgatgctagctctgccattaatagaccacaactcacatttgtgaagaattttattactgccaatccggcatggagtaaggcagtctttatttcgccttacaattcacagaatgctgtgtctcgttcaatgctgggtcttaccactcagactgttgattcctcacagggttcagaataccagtacgttatcttctgtcaaacagcagatacggcacatgctaacaacattaacagatttaatgttgcaatcactcgtgcccaaaaaggtattctttgtgttatgacatctcaggcactctttgagtccttagagtttactgaattgtcttttactaattacaagctccagtctcagattgtaactggcctttttaaagattgctctagagaaacttctggcctctcacctgcttatgcaccaacatatgttagtgttgatgacaagtataagacgagtgatgagctttgcgtgaatcttaatttacccgcaaatgtcccatactctcgtgttatttccaggatgggctttaaactcgatgcaacagttcctggatatcctaagcttttcattactcgtgaagaggctgtaaggcaagttcgaagctggataggcttcgatgttgagggtgctcatgcttcccgtaatgcatgtggcaccaatgtgcctctacaattaggattttcaactggtgtgaactttgttgttcagccagttggtgttgtagacactgagtggggtaacatgttaacgggcattgctgcacgtcctccaccaggtgaacagtttaagcacctcgtgcctcttatgcataagggggctgcgtggcctattgttagacgacgtatagtgcaaatgttgtcagacactttagacaaattgtctgattactgtacgtttgtttgttgggctcatggctttgaattaacgtctgcatcatacttttgcaagataggtaaggaacagaagtgttgcatgtgcaatagacgcgctgcagcgtactcttcacctctgcaatcttatgcctgctggactcattcctgcggttatgattatgtctacaaccctttctttgtcgatgttcaacagtggggttatgtaggcaatcttgctactaatcacgatcgttattgctctgtccatcaaggagctcatgtggcttctaatgatgcaataatgactcgttgtttagctattcattcttgttttatagaacgtgtggattgggatatagagtatccttatatctcacatgaaaagaaattgaattcctgttgtagaatcgttgagcgcaacgtcgtacgtgctgctcttcttgccggttcatttgacaaagtctatgatattggcaatcctaaaggaattcctattgttgatgaccctgtggttgattggcattattttgatgcacagcccttgaccaggaaggtacaacagcttttctatacagaggacatggcctcaagatttgctgatgggctctgcttattttggaactgtaatgtaccaaaatatcctaataatgcaattgtatgcaggtttgacacacgtgtgcattctgagttcaatttgccaggttgtgatggcggtagtttgtatgttaacaagcacgcttttcatacaccagcatatgatgtgagtgcattccgtgatctgaaacctttaccattcttttattattctactacaccatgtgaagtgcatggtaatggtagtatgatagaggatattgattatgtacccctaaaatctgcagtctgtattacagcttgtaatttagggggcgctgtttgtaggaagcatgctacagagtacagagagtatatggaagcatataatcttgtctctgcatcaggtttccgcctttggtgttataagacctttgatatttataatctctggtctacttttacaaaagttcaaggtttggaaaacattgcttttaatgttgttaaacaaggccattttattggtgttgagggtgaactacctgtagctgtagtcaatgataagatcttcaccaagagtggcgttaatgacatttgtatgtttgagaataaaaccactttgcctactaatatagcttttgaactctatgctaagcgtgctgtacgctcgcatcccgatttcaaattgctacacaatttacaagcagacatttgctacaagttcgtcctttgggattatgaacgtagcaatatttatggtactgctactattggtgtatgtaagtacactgatattgatgttaattcagctttgaatatatgttttgacatacgcgataattgttcattggagaagttcatgtctactcccaatgccatctttatttctgatagaaaaatcaagaaatacccttgtatggtaggtcctgattatgcttacttcaatggtgctatcatccgtgatagtgatgttgttaaacaaccagtgaagttctacttgtataagaaagtcaataatgagtttattgatcctactgagtgtatttacactcagagtcgctcttgtagtgacttcctacccctttctgacatggagaaagactttctatcttttgatagtgatgttttcattaagaagtatggcttggaaaactatgcttttgagcacgtagtctatggagacttctctcatactacgttaggcggtcttcacttgcttattggtttatacaagaagcaacaggaaggtcatattattatggaagaaatgctaaaaggtagctcaactattcataactattttattactgagactaacacagcggcttttaaggcggtgtgttctgttatagatttaaagcttgacgactttgttatgattttaaagagtcaagaccttggcgtagtatccaaggttgtcaaggttcctattgacttaacaatgattgagtttatgttatggtgtaaggatggacaggttcaaaccttctaccctcgactccaggcttctgcagattggaaacctggtcatgcaatgccatccctctttaaagttcaaaatgtaaaccttgaacgttgtgagcttgctaattacaagcaatctattcctatgcctcgcggtgtgcacatgaacatcgctaaatatatgcaattgtgccagtatttaaatacttgcacattagccgtgcctgccaatatgcgtgttatacattttggcgctggttctgataaaggtatcgctcctggtacctcagttttacgacagtggcttcctacagatgccattattatagataatgatttaaatgagttcgtgtcagatgctgacataactttatttggagattgtgtaactgtacgtgtcggccaacaagtggatcttgttatttccgacatgtatgatcctactactaagaatgtaacaggtagtaatgagtcaaaggctttattctttacttacctgtgtaacctcattaataataatcttgctcttggtgggtctgttgctattaaaataacagaacactcttggagcgttgaactttatgaacttatgggaaaatttgcttggtggactgttttctgcaccaatgcaaatgcatcctcatctgaaggattcctcttaggtattaattacttgggtactattaaagaaaatatagatggtggtgctatgcacgccaactatatattttggagaaattccactcctatgaatctgagtacttactcactttttgatttatccaagtttcaattaaaattaaaaggaacaccagttcttcaattaaaggagagtcaaattaacgaactcgtaatatctctcctgtcgcagggtaagttacttatccgtgacaatgatacactcagtgtttctactgatgttcttgttaacacctacagaaagttacgttgatgtagggccagattctgttaagtctgcttgtattgaggttgatatacaacagactttctttgataaaacttggcctaggccaattgatgtttctaaggctgacggtattatataccctcaaggccgtacatattctaacataactatcacttatcaaggtctttttccctatcagggagaccatggtgatatgtatgtttactctgcaggacatgctacaggcacaactccacaaaagttgtttgtagctaactattctcaggacgtcaaacagtttgctaatgggtttgtcgtccgtataggagcagctgccaattccactggcactgttattattagcccatctaccagcgctactatacgaaaaatttaccctgcttttatgctgggttcttcagttggtaatttctcagatggtaaaatgggccgcttcttcaatcatactctagttcttttgcccgatggatgtggcactttacttagagctttttattgtattctagagcctcgctctggaaatcattgtcctgctggcaattcctatacttcttttgccacttatcacactcctgcaacagattgttctgatggcaattacaatcgtaatgccagtctgaactcttttaaggagtattttaatttacgtaactgcacctttatgtacacttataacattaccgaagatgagattttagagtggtttggcattacacaaactgctcaaggtgttcacctcttctcatctcggtatgttgatttgtacggcggcaatatgtttcaatttgccaccttgcctgtttatgatactattaagtattattctatcattcctcacagtattcgttctatccaaagtgatagaaaagcttgggctgccttctacgtatataaacttcaaccgttaactttcctgttggatttttctgttgatggttatatacgcagagctatagactgtggttttaatgatttgtcacaactccactgctcatatgaatccttcgatgttgaatctggagtttattcagtttcgtctttcgaagcaaaaccttctggctcagttgtggaacaggctgaaggtgttgaatgtgatttttcacctcttctgtctggcacacctcctcaggtttataatttcaagcgtttggtttttaccaattgcaattataatcttaccaaattgctttcacttttttctgtgaatgattttacttgtagtcaaatatctccagcagcaattgctagcaactgttattcttcactgattttggattacttttcatacccacttagtatgaaatccgatctcagtgttagttctgctggtccaatatcccagtttaattataaacagtccttttctaatcccacatgtttgattttagcgactgttcctcataaccttactactattactaagcctcttaagtacagctatattaacaagtgctctcgtcttctttctgatgatcgtactgaagtacctcagttagtgaacgctaatcaatactcaccctgtgtatccattgtcccatccactgtgtgggaagacggtgattattataggaaacaactatctccacttgaaggtggtggctggcttgttgctagtggctcaactgttgccatgactgagcaattacagatgggctttggtattacagttcaatatggtacagacaccaatagtgtttgccccaagcttgaatttgctaatgacacaaaaattgcctctcaattaggcaattgcgtggaatattccctctatggtgtttcgggccgtggtgtttttcagaattgcacagctgtaggtgttcgacagcagcgctttgtttatgatgcgtaccagaatttagttggctattattctgatgatggcaactactactgtttgcgtgcttgtgttagtgttcctgtttctgtcatctatgataaagaaactaaaacccacgctactctatttggtagtgttgcatgtgaacacatttcttctaccatgtctcaatactcccgttctacgcgatcaatgcttaaacggcgagattctacatatggcccccttcagacacctgttggttgtgtcctaggacttgttaattcctctttgttcgtagaggactgcaagttgcctcttggtcaatctctctgtgctcttcctgacacacctagtactctcacacctcgcagtgtgcgctctgttccaggtgaaatgcgcttggcatccattgcttttaatcatcctattcaggttgatcaacttaatagtagttattttaaattaagtatacccactaatttttcctttggtgtgactcaggagtacattcagacaaccattcagaaagttactgttgattgtaaacagtacgtttgcaatggtttccagaagtgtgagcaattactgcgcgagtatggccagttttgttccaaaataaaccaggctctccatggtgccaatttacgccaggatgattctgtacgtaatttgtttgcgagcgtgaaaagctctcaatcatctcctatcataccaggttttggaggtgactttaatttgacacttctagaacctgtttctatatctactggcagtcgtagtgcacgtagtgctattgaggatttgctatttgacaaagtcactatagctgatcctggttatatgcaaggttacgatgattgcatgcagcaaggtccagcatcagctcgtgatcttatttgtgctcaatatgtggctggttacaaagtattacctcctcttatggatgttaatatggaagccgcgtatacttcatctttgcttggcagcatagcaggtgttggctggactgctggcttatcctcctttgctgctattccatttgcacagagtatcttttataggttaaacggtgttggcattactcaacaggttctttcagagaaccaaaagcttattgccaataagtttaatcaggctctgggagctatgcaaacaggcttcactacaactaatgaagcttttcagaaggttcaggatgctgtgaacaacaatgcacaggctctatccaaattagctagcgagctatctaatacttttggtgctatttccgcctctattggagacatcatacaacgtcttgatgttctcgaacaggacgcccaaatagacagacttattaatggccgtttgacaacactaaatgcttttgttgcacagcagcttgttcgttccgaatcagctgctctttccgctcaattggctaaagataaagtcaatgagtgtgtcaaggcacaatccaagcgttctggattttgcggtcaaggcacacatatagtgtcctttgttgtaaatgcccctaatggcctttacttcatgcatgttggttattaccctagcaaccacattgaggttgtttctgcttatggtctttgcgatgcagctaaccctactaattgtatagcccctgttaatggctactttattaaaactaataacactaggattgttgatgagtggtcatatactggctcgtccttctatgcacctgagcccattacctcccttaatactaagtatgttgcaccacaggtgacataccaaaacatttctactaacctccctcctcctcttctcggcaattccaccgggattgacttccaagatgagttggatgagtttttcaaaaatgttagcaccagtatacctaattttggttccctaacacagattaatactacattactcgatcttacctacgagatgttgtctcttcaacaagttgttaaagcccttaatgagtcttacatagaccttaaagagcttggcaattatacttattacaacaaatggccgtggtacatttggcttggtttcattgctgggcttgttgccttagctctatgcgtcttcttcatactgtgctgcactggttgtggcacaaactgtatgggaaaacttaagtgtaatcgttgttgtgatagatacgaggaatacgacctcgagccgcataaggttcatgttcactaattaacgaactattaatgagagttcaaagaccacccactctcttgttagtgttttcactctctcttttggtcactgcatcctcaaaacctctctatgtacctgagcattgtcagaattattctggttgcatgcttagggcttgtattaaaactgcccaagctgatacagctggtctttatacaaattttcgaattgacgtcccatctgcagaatcaactggtactcaatcagtttctgtcgatcttgagtcaacttcaactcatgatggtcctaccgaacatgttactagtgtgaatctttttgacgttggttactcagttaattaacgaactctatggattacgtgtctctgcttaatcaaatttggcagaagtaccttaactcaccgtatactacttgtttgtacatccctaaacccacagctaagtatacacctttagttggcacttcattgcaccctgtgctgtggaactgtcagctatcctttgctggttatactgaatctgctgttaattctacaaaagctttggccaaacaggacgcagctcagcgaatcgcttggttgctacataaggatggaggaatccctgatggatgttccctctacctccggcactcaagtttattcgcgcaaagcgaggaagaggagccattctccaactaagaaactgcgctacgttaagcgtagattttctcttctgcgccatgaagaccttagtgttattgtccaaccaacacactatgtcagggttacattttcagaccccaacatgtggtatctacgttcgggtcatcatttacactcagttcacaattggcttaaaccttatggcggccaacctgtttctgagtaccatattactctagctttgctaaatctcactgatgaagatttagctagagatttttcacccattgcgctctttttgcgcaatgtcagatttgagctacatgagttcgccttgctgcgcaaaactcttgttcttaatgcatcagagatctactgtgctaacatacatagatttaagcctgtgtatagagttaacacggcaatccctactattaaggattggcttctcgttcagggattttccctttaccatagtggcctccctttacatatgtcaatctctaaattgcatgcactggatgatgttactcgcaattacatcattacaatgccatgctttagaacttaccctcaacaaatgtttgttactcctttggccgtagatgttgtctccatacggtcttccaatcagggtaataaacaaattgttcattcttatcccattttacatcatccaggattttaacgaactatggctttctcggcgtctttatttaaacccgtccagctagtcccagtttctcctgcatttcatcgcattgagtctactgactctattgttttcacatacattcctgctagcggctatgtagctgctttagctgtcaatgtgtgtctcattcccctattattactgctacgtcaagatacttgtcgtcgcagcattatcagaactatggttctctatttccttgttctgtataactttttattagccattgtactagtcaatggtgtacattatccaactggaagttgcctgatagccttcttagttatcctcataatactttggtttgtagatagaattcgtttctgtctcatgctgaattcctacattccactgtttgacatgcgttcccactttattcgtgttagtacagtttcttctcatggtatggtccctgtaatacacaccaaaccattatttattagaaacttcgatcagcgttgcagctgttctcgttgtttttatttgcactcttccacttatatagagtgcacttatattagccgttttagtaagattagcctagtttctgtaactgacttctccttaaacggcaatgtttccactgttttcgtgcctgcaacgcgcgattcagttcctcttcacataatcgccccgagctcgcttatcgtttaagcagctctgcgctactatgggtcccgtgtagaggctaatccattagtctctctttggacatatggaaaacgaactatgttaccctttgtccaagaacgaatagggttgttcatagtaaactttttcatttttaccgtagtatgtgctataacactcttggtgtgtatggctttccttacggctactagattatgtgtgcaatgtatgacaggcttcaataccctgttagttcagcccgcattatacttgtataatactggacgttcagtctatgtaaaattccaggatagtaaaccccctctaccacctgacgagtgggtttaacgaactccttcataatgtctaatatgacgcaactcactgaggcgcagattattgccattattaaagactggaactttgcatggtccctgatctttctcttaattactatcgtactacagtatggatacccatcccgtagtatgactgtctatgtctttaaaatgtttgttttatggctcctatggccatcttccatggcgctatcaatatttagcgccgtttatccaattgatctagcttcccagataatctctggcattgtagcagctgtttcagctatgatgtggatttcctactttgtgcagagtatccggctgtttatgagaactggatcatggtggtcattcaatcctgagactaattgccttttgaacgttccatttggtggtacaactgtcgtacgtccactcgtagaggactctaccagtgtaactgctgttgtaaccaatggccacctcaaaatggctggcatgcatttcggtgcttgtgactacgacagacttcctaatgaagtcaccgtggccaaacccaatgtgctgattgctttaaaaatggtgaagcggcaaagctacggaactaattccggcgttgccatttaccatagatataaggcaggtaattacaggagtccgcctattacggcggatattgaacttgcattgcttcgagcttaggctctttagtaagagtatcttaattgattttaacgaatctcaatttcattgttatggcatcccctgctgcacctcgtgctgtttcctttgccgataacaatgatataacaaatacaaacctatctcgaggtagaggacgtaatccaaaaccacgagctgcaccaaataacactgtctcttggtacactgggcttacccaacacgggaaagtccctcttacctttccacctgggcagggtgtacctcttaatgccaattctacccctgcgcaaaatgctgggtattggcggagacaggacagaaaaattaataccgggaatggaattaagcaactggctcccaggtggtacttctactacactggaactggacccgaagcagcactcccattccgggctgttaaggatggcatcgtttgggtccatgaagatggcgccactgatgctccttcaacttttgggacgcggaaccctaacaatgattcagctattgttacacaattcgcgcccggtactaagcttcctaaaaacttccacattgaggggactggaggcaatagtcaatcatcttcaagagcctctagcttaagcagaaactcttccagatctagttcacaaggttcaagatcaggaaactctacccgcggcacttctccaggtccatctggaatcggagcagtaggaggtgatctactttaccttgatcttctgaacagactacaagcccttgagtctggcaaagtaaagcaatcgcagccaaaagtaatcactaagaaagatgctgctgctgctaaaaataagatgcgccacaagcgcacttccaccaaaagtttcaacatggtgcaagcttttggtcttcgcggaccaggagacctccagggaaactttggtgatcttcaattgaataaactcggcactgaggacccacgttggccccaaattgctgagcttgctcctacagccagtgcttttatgggtatgtcgcaatttaaacttacccatcagaacaatgatgatcatggcaaccctgtgtacttccttcggtacagtggagccattaaacttgacccaaagaatcccaactacaataagtggttggagcttcttgagcaaaatattgatgcctacaaaaccttccctaagaaggaaaagaaacaaaaggcaccaaaagaagaatcaacagaccaaatgtctgaacctccaaaggagcagcgtgtgcaaggtagcatcactcagcgcactcgcacccgtccaagtgttcagcctggtccaatgattgatgttaacactgattagtgtcactcaaagtaacaagatcgcggcaatcgtttgtgtttggcaaccccatctcaccatcgcttgtccactcttgcacagaatggaatcatgttgtaattacagtgcaataaggtaattataacccatttaattgatagctatgctttattaaagtgtgtagctgtagagagaatgttaaagactgtcacctctgcttgattgcaagtgaacagtgccccccgggaagagctctacagtgtgaaatgtaaataaaaaatagctattattcaattagattaggctaattagatgatttgcaaaaaaaaaaaa

(19) United States
(12) Patent Application Publication
(10) Pub. No.: US 2024/0285561 A1
(43) Pub. Date: Aug. 29, 2024

(54) METHODS OF USING PROBENECID FOR TREATMENT OF CORONAVIRUS INFECTIONS

(71) Applicant: University of Georgia Research Foundation, Inc., Athens, GA (US)

(72) Inventors: Ralph A. Tripp, Watkinsville, GA (US); Jackelyn Murray, Monroe, GA (US); Robert Jeff Hogan, Watkinsville, GA (US)

(21) Appl. No.: 18/443,918

(22) Filed: Feb. 16, 2024

Related U.S. Application Data
(63) Continuation of application No. 17/465,932, filed on Sep. 3, 2021, now Pat. No. 11,903,916.
Application No. 17/465,932 is a continuation of application No. 16/875,487, filed on May 15, 2020, now Pat. No. 11,116,737.
(60) Provisional application No. 63/008,624, filed on Apr. 10, 2020.
(60) Provisional application No. 63/023,021, filed on May 11, 2020.

(51) Int. Cl. A61K 31/195; A61K 9/00; A61P 31/14

(52) U.S. Cl. CPC A61K 31/195; A61P 31/14; A61K 9/0019; A61K 9/0053

(57) ABSTRACT

Compositions and methods of treating a subject for a coronavirus infection are provided. The methods typically include administering the subject an effective amount of probenecid, a metabolite or anaolg thereof, or a pharmaceutically acceptable salt thereof. The methods can by therapeutic and/or prophylactic. The amount of probenecid or a pharmaceutically acceptable salt thereof can be effective to, for example, reduce viral replication, reduce one or more symptoms of disease, disorder, or illness associated with virus, or a combination thereof. In preferred embodiments, the virus is a Severe acute respiratory syndrome-related coronavirus such as SARS-CoV-2 or SARS-CoV, a Middle East respiratory syndrome-related coronavirus such as MERS-CoV, or a coronavirus that causes the common cold.

### CROSS REFERENCE TO RELATED APPLICATIONS

This application is a continuation of U.S. application Ser. No. 17/465,932 filed Sep. 3, 2021, which is a continuation of U.S. application Ser. No. 16/875,487, filed May 15, 2020, now U.S. Pat. No. 11,116,737, issued Sep. 14, 2021, which claims benefit of and priority to U.S. Provisional Application No. 63/008,624, filed Apr. 10, 2020, and U.S. Provisional Application No. 63/023,021, filed May 11, 2020, each of which is specifically incorporated by reference herein in its entirety.

### REFERENCE TO SEQUENCE LISTING

The Sequence Listing XML submitted as a file named “UGA_2020_148_21_CON2_ST26.xml” created on May 6, 2024 and having a size of 124,427 bytes is hereby incorporated by reference pursuant to 37 C.F.R. § 1.834(c)(1).

### FIELD OF THE INVENTION

The field of the invention generally related to compositions and methods for the treatment of coronavirus-related illnesses.

### BACKGROUND OF THE INVENTION

A seventh human coronavirus was recently identified in Wuhan, China (Coronaviridae Study Group of the International Committee on Taxonomy of Viruses, Nat Microbiol 2020. DOI: 10.1038/s41564-020-0695-z, WHO “Pneumonia of unknown cause—China;” World Health Organization: Online, 2020). Initially described as COVID-19 after its discovery in December 2019, this virus has now been classified as a betacoronavirus within the same species as the Severe acute respiratory syndrome coronavirus (SARS-CoV), which was responsible for a pandemic in 2002-2003 (Coronaviridae Study Group of the International Committee on Taxonomy of Viruses, Nat Microbiol 2020. DOI: 10.1038/s41564-020-0695-z, Ratia, et al., Proc Natl Acad Sci U S A 2008, 105 (42), 16119-24. DOI: 10.1073/pnas.0805240105). Hence, COVID-19 has now been classified as SARS-CoV-2. Although SARS-CoV-2 does not appear to be lethal as SARS-CoV, it has rapidly spread worldwide according to a World Health Organization situation report. The rapid spread of SARS-CoV-2 and its ability to cause death particular in older individuals, or individuals with underlying conditions, has created an urgency for the need of antiviral therapeutics and vaccines for use against the virus (“CDC People at Risk for Serious Illness from COVID-19,” CDC website).

Thus, it is an object of the invention to provide compositions and methods of treating infections caused by coronaviruses, particularly coronaviruses that cause severe acute respiratory syndrome, including, but limited to SARS-CoV-2.

### SUMMARY OF THE INVENTION

Compositions and methods of treating a subject for a coronavirus infection are provided. The methods typically include administering the subject an effective amount of probenecid, a metabolite or analog thereof, or a pharmaceutically acceptable salt thereof. The amount of probenecid, metabolite or analog thereof, or a pharmaceutically acceptable salt thereof can be effective to, for example, reduce viral replication, reduce one or more symptoms of a disease, disorder, or illness associated with virus, or a combination thereof. Symptoms include, but are not limited to, fever, congestion in the nasal sinuses and/or lungs, runny or stuffy nose, cough, sneezing, sore throat, body aches, fatigue, shortness of breath, chest tightness, wheezing when exhaling, chills, muscle aches, headache, diarrhea, tiredness, nausea, vomiting, and combinations thereof. The subject can be, for example, a mammal or a bird. In preferred embodiments, the subject is a human.

The subject can be symptomatic or asymptomatic. In some embodiments, the subject has been, or will be, exposed to the virus. In some embodiments, treatment begins 1, 2, 3, 4, 5, or more hours, days, or weeks prior to or after exposure to the virus. In some embodiments, the subject has not been exposed to the virus. In some embodoiments, the subject anticipates being exposed to the virus. Thus, preventative and prophylactic methods are also provided.

The virus can be a Severe acute respiratory syndrome-related coronavirus, a Bat Hp-betacoronavirus Zhejiang2013, a Rousettus bat coronavirus GCCDC1, a Rousettus bat coronavirus HKU9, Eidolon bat coronavirus C704, a Pipistrellus bat coronavirus HKU5, a Tylonycteris bar coronovirus HKU4, a Middle East respiratory syndrome-related coronavirus, a Hedgehog coronavirus, a murine coronavirus, a Human coronavirus HKU1, a China Rattus coronavirus HKU24, a Betacoronavirus 1, a Myodes coronavirus 2JL14, a Human coronavirus NL63, a Human coronavirus 229E, or a Human coronavirus OC43.

In preferred embodiments, the virus is a Severe acute respiratory syndrome-related coronavirus, such as SARS-CoV-2, SARS-CoV, SARS-CoV RaTG13, SARS-CoV PC4-227, or SARSr-CoV BtKY72.

In some embodiments, the virus is a SARS-CoV-2 having a genome encoded by a nucleic acid sequence having at least 85%, 86%, 87%, 88%, 89%, 90%, 91%, 92%, 93%, 94%, 95%, 96%, 97%, 98%, 99%, or 100% sequence identity SEQ ID NO:1 or 2.

In some embodiments, the Severe acute respiratory syndrome-related coronavirus is SARS-CoV, for example, a SAR-CoV having a genome encoded by a nucleic acid sequence having at least 85%, 86%, 87%, 88%, 89%, 90%, 91%, 92%, 93%, 94%, 95%, 96%, 97%, 98%, 99%, or 100% sequence identity SEQ ID NO:3.

In some embodiments, the virus is a Middle East respiratory syndrome-related coronavirus, for example, a MERS-CoV having a genome encoded by a nucleic acid sequence having at least 85%, 86%, 87%, 88%, 89%, 90%, 91%, 92%, 93%, 94%, 95%, 96%, 97%, 98%, 99%, or 100% sequence identity SEQ ID NO:4.

In some embodiments, the subject has a disease or disorder associated with the virus. For example, in embodiments, a subject exposed or infected with SARS-CoV-2 has COVID-19.

The probenecid, metabolite or analog thereof, or pharmaceutically acceptable salt thereof is typically administered in a pharmaceutical composition including a pharmaceutically acceptable carrier and/or excipient. Thus, pharmaceutical compositions are also provided. Dosage forms are also provided and include, but not limited to 500 mg tablets of probenecid, a metabolite or analog thereof, or pharmaceutically acceptable salt thereof. In some embodiments, the subject is administered a 10 mg-1,000 mg or, 50 mg-500 mg dose of probenecid, metabolite or analog thereof, or pharmaceutically acceptable salt thereof 1, 2, 3, 4, or 5 times per day. In some embodiments, the dosage regimen is a pulse dosage regimen that include 1, 2, 3, or more large bolus doses in close proximity (e.g., minutes or hours apart). In some embodiments, the bolus doses are followed by a drug administration holiday, optionally until the drug level in the subject's serum drops to zero or near zero.

The probenecid, a metabolite or analog thereof, or a pharmaceutically acceptable salt thereof can be administered systemically or locally. Exemplary routes of administration include, but are not limited to, oral, parenteral, topical or mucosal. In some embodiments, the composition is administered to lungs (e.g., pulmonary administration) by oral inhalation or intranasal administration. In some embodiments, the composition is administered intranasally to the nasal mucosa.

### BRIEF DESCRIPTION OF THE DRAWINGS

FIG. 1 is a bar graph showing (left-to-right) the effect of probenecid (5 μM, 2.5 μM, 1 μM, or 0.1 μM) compared to controls (DMSO (infected), DMSO (only)) on viral replication using a plaque reduction assay.

FIG. 2 is a bar graph showing (left-to-right) the effect of probenecid pre-treatment (5 μM, 2.5 μM, 1 μM, 0.1 μM 0.01 μM, 0.001 μM, or 0.0001 μM) compared to controls (DMSO (infected), DMSO (only)) on viral replication using a plaque reduction assay.

FIG. 3 is a bar graph showing (left-to-right) the effect of probenecid post-treatment (5 μM, 2.5 μM, 1 μM, 0.1 μM 0.01 μM, 0.001 μM, or 0.0001 μM) compared to controls (DMSO (infected), DMSO (only)) on viral replication using a plaque reduction assay.

### DETAILED DESCRIPTION OF THE INVENTION

### I. Definitions

As used herein, the terms “individual”, “host”, “subject”, and “patient” are used interchangeably herein, and refer animals, particularly birds and mammals, including, but not limited to, primates such as humans, bats, rodents, such as mice and rats, and other laboratory animals.

As used herein the term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease, and the treatment being administered.

As used herein, the term “carrier” or “excipient” refers to an organic or inorganic ingredient, natural or synthetic inactive ingredient in a formulation, with which one or more active ingredients are combined.

As used herein, the term “pharmaceutically acceptable” means a non-toxic material that does not interfere with the effectiveness of the biological activity of the active ingredients.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values may range in value either above or below the stated value in a range of approx. +/−5%; in other forms the values may range in value either above or below the stated value in a range of approx. +/−2%; in other forms the values may range in value either above or below the stated value in a range of approx. +/−1%. The preceding ranges are intended to be made clear by context, and no further limitation is implied.

### II. Compositions

### A. Probenecid, Metabolites, Analogs, and Pharmaceutically Acceptable Salts Thereof

The disclosed methods include administering a subject in need thereof an effective amount of probenecid, a metabolite or analog thereof, or a pharmaceutically acceptable salt thereof, including, but not limited to a sodium salt thereof.

Probenecid (4-[(dipropylamino) sulfony 1] benzoic acid (CAS No. 57-66-9)) has the structure:

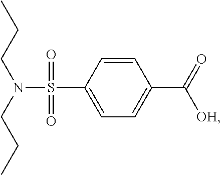 [SMILES: CCCN(CCC)S(=O)(=O)c1ccc(C(=O)O)cc1]

and has been sold under the brand names BENEMID and PROBALAN.

Probenecid is a highly lipid soluble benzoic acid derivative with an excellent safety profile that was developed in the 1950's to decrease the renal tubular excretion of penicillin. Probenecid, USP is a white or nearly white, fine, crystalline powder. Probenecid is soluble in dilute alkali, in alcohol, in chloroform, and in acetone; it is practically insoluble in water and in dilute acids. It has a half life of 6-12 hours. See also Drugbank Accession Number DB01032 (APRD00167).

Metabolites and analogs of probenecid are known, see, for example, Guarino, et al., “Mass spectral identification of probenecid metabolites in rat bile,” Eur. J. Pharmacol., 8, 244-252 (1969), Perel, et al., “Identification and renal excretion of probenecid metabolites in man,” Life Sciences, 9, 23, 1337-1343 (1970), Perel, et al., “Studies of the renal excretion of probenecid acyl glucuronide in man,” Eur. J. Clin. Pharmacol, 3, 106-112 (1971), Dayton and Perel, “The metabolism of probenecid in man,”. N. Y. Acad. Sci., 179, 399-402 (1971), Dayton, et al., “The effect of probenecid, phenylbutazone and their analogues on the excretion of L-ascorbic acid in rats,” J. Med. Chem. 9, 941-944 (1966), and Israili, et al., “Metabolites of probenecid. Chemical, physical, and pharmacological studies,” J. Med. Chem., 15, 7, 709-713 (1972), each of which is specifically incorporated by reference in its entirety.

In some embodiments, the metabolite is a glucuronide derivative of probenecid such as acyl glucuronide or a β-ether glucuronide.

Exemplary probenecid metabolites and analogs include, but are not limited to,

dl-p-(N-Propy-N-2-hydroxypropylsulfamoyl)benzoic Acid,

Propylaminopropyl Acetate,

Piopylaminopropan-3-ol,

p-(N-Propyl-N-3-hydroxypropylsulfamoyl)benzoic Acid,

Propylaminopropionitrile,

p-(N-Propyl-N-3-propionitrilosulfamoyl)benzoic Acid,

p-(N-Propyl-N-2-carboxyethylsulfamoyl)benzoic Acid,

p-(N-Propylsulfamoyl)benzoic Acid,

p-(N,N-Pentamethylenesulfamoyl)benzoic Acid (Piperidyl

Analog),

p-(N-Propyl-N-2-propenylsulfamoyl)benzoic Acid, and

p-(N-Propyl-N-2-oxopropylsulfamoyl)benzoic Acid.

Typically the metabolite or analog can on its own, or upon further metabolism thereof by a subject, treat a coronavirus when administered in an effective amount as discussed herein. For example, in some embodiments, the metabolite or analog can on its own, or upon further metabolism thereof by a subject, reduce viral replication.

### B. Formulations

Probenecid, metabolites and analogs thereof, and pharmaceutically acceptable salts thereof can be formulated in a pharmaceutical composition. Pharmaceutical compositions can be for administration by parenteral (intramuscular, intraperitoneal, intravenous (IV) or subcutaneous injection), enteral, transdermal (either passively or using iontophoresis or electroporation), or transmucosal (nasal, pulmonary, vaginal, rectal, or sublingual) routes of administration or using bioerodible inserts and can be formulated in dosage forms appropriate for each route of administration.

The compositions can be administered systemically.

The compositions can be formulated for immediate release, extended release, or modified release. A delayed release dosage form is one that releases a drug (or drugs) at a time other than promptly after administration. An extended release dosage form is one that allows at least a twofold reduction in dosing frequency as compared to that drug presented as a conventional dosage form (e.g. as a solution or prompt drug-releasing, conventional solid dosage form). A modified release dosage form is one for which the drug release characteristics of time course and/or location are chosen to accomplish therapeutic or convenience objectives not offered by conventional dosage forms such as solutions, ointments, or promptly dissolving dosage forms. Delayed release and extended release dosage forms and their combinations are types of modified release dosage forms.

Formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes, but is not limited to, diluents, binders, lubricants, desintegrators, fillers, and coating compositions.

“Carrier” also includes all components of the coating composition which may include plasticizers, pigments, colorants, stabilizing agents, and glidants. The delayed release dosage formulations may be prepared as described in references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6^{th} Edition, Ansel et.al., (Media, PA: Williams and Wilkins, 1995) which provides information on carriers, materials, equipment and process for preparing tablets and capsules and delayed release dosage forms of tablets, capsules, and granules.

The compound can be administered to a subject with or without the aid of a delivery vehicle. Appropriate delivery vehicles for the compounds are known in the art and can be selected to suit the particular active agent. For example, in some embodiments, the active agent(s) is incorporated into or encapsulated by, or bound to, a nanoparticle, microparticle, micelle, synthetic lipoprotein particle, or carbon nanotube. For example, the compositions can be incorporated into a vehicle such as polymeric microparticles which provide controlled release of the active agent(s). In some embodiments, release of the drug(s) is controlled by diffusion of the active agent(s) out of the microparticles and/or degradation of the polymeric particles by hydrolysis and/or enzymatic degradation.

Suitable polymers include ethylcellulose and other natural or synthetic cellulose derivatives. Polymers which are slowly soluble and form a gel in an aqueous environment, such as hydroxypropyl methylcellulose or polyethylene oxide, may also be suitable as materials for drug containing microparticles or particles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof. In some embodiments, both agents are incorporated into the same particles and are formulated for release at different times and/or over different time periods. For example, in some embodiments, one of the agents is released entirely from the particles before release of the second agent begins. In other embodiments, release of the first agent begins followed by release of the second agent before the all of the first agent is released. In still other embodiments, both agents are released at the same time over the same period of time or over different periods of time.

### 1. Oral Immediate Release Formulations

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit® (Roth Pharma, Westerstadt, Germany), Zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants.

Diluents, also termed “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powder sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydorxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, POLOXAMER® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, the tablets, beads granules or particles may also contain minor amount of nontoxic auxiliary substances such as wetting or emulsifying agents, dyes, pH buffering agents, and preservatives.

### 2. Extended Release Dosage Forms

The extended release formulations are generally prepared as diffusion or osmotic systems, for example, as described in “Remington—The science and practice of pharmacy” (20th ed., Lippincott Williams & Wilkins, Baltimore, MD, 2000). A diffusion system typically consists of two types of devices, reservoir and matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but not limited to, methyl acrylate-methyl methacrylate, polyvinyl chloride, and polyethylene. Hydrophilic polymers include, but are not limited to, methylcellulose, hydroxypropylcellulose, hydroxypropylmethylcellulose, sodium carboxymethylcellulose, and carbopol 934, polyethylene oxides. Fatty compounds include, but are not limited to, various waxes such as carnauba wax and glyceryl tristearate.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above could be combined in a final dosage form comprising single or multiple units. Examples of multiple units include multilayer tablets, capsules containing tablets, beads, granules, etc.

An immediate release portion can be added to the extended release system by means of either applying an immediate release layer on top of the extended release core using coating or compression process or in a multiple unit system such as a capsule containing extended and immediate release beads.

Extended release tablets containing hydrophilic polymers are prepared by techniques commonly known in the art such as direct compression, wet granulation, or dry granulation processes. Their formulations usually incorporate polymers, diluents, binders, and lubricants as well as the active pharmaceutical ingredient. The usual diluents include inert powdered substances such as any of many different kinds of starch, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidine can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In a congealing method, the drug is mixed with a wax material and either spray-congealed or congealed and screened and processed.

### 3. Delayed Release Dosage Forms

Delayed release formulations are created by coating a solid dosage form with a film of a polymer which is insoluble in the acid environment of the stomach, and soluble in the neutral environment of small intestines.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename EUDRAGIT®. (Rohm Pharma; Westerstadt, Germany), including EUDRAGIT®. L30D-55 and L100-55 (soluble at pH 5.5 and above), EUDRAGIT®. L-100 (soluble at pH 6.0 and above), EUDRAGIT®. S (soluble at pH 7.0 and above, as a result of a higher degree of esterification), and EUDRAGITS®. NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

### Methods of Manufacturing

As will be appreciated by those skilled in the art and as described in the pertinent texts and literature, a number of methods are available for preparing drug-containing tablets, beads, granules or particles that provide a variety of drug release profiles. Such methods include, but are not limited to, the following: coating a drug or drug-containing composition with an appropriate coating material, typically although not necessarily incorporating a polymeric material, increasing drug particle size, placing the drug within a matrix, and forming complexes of the drug with a suitable complexing agent.

The delayed release dosage units may be coated with the delayed release polymer coating using conventional techniques, e.g., using a conventional coating pan, an airless spray technique, fluidized bed coating equipment (with or without a Wurster insert). For detailed information concerning materials, equipment and processes for preparing tablets and delayed release dosage forms, see Pharmaceutical Dosage Forms: Tablets, eds. Lieberman et al. (New York: Marcel Dekker, Inc., 1989), and Ansel et al., Pharmaceutical Dosage Forms and Drug Delivery Systems, 6.sup.th Ed. (Media, PA: Williams & Wilkins, 1995).

A preferred method for preparing extended release tablets is by compressing a drug-containing blend, e.g., blend of granules, prepared using a direct blend, wet-granulation, or dry-granulation process. Extended release tablets may also be molded rather than compressed, starting with a moist material containing a suitable water-soluble lubricant. However, tablets are preferably manufactured using compression rather than molding. A preferred method for forming extended release drug-containing blend is to mix drug particles directly with one or more excipients such as diluents (or fillers), binders, disintegrants, lubricants, glidants, and colorants. As an alternative to direct blending, a drug-containing blend may be prepared by using wet-granulation or dry-granulation processes. Beads containing the active agent may also be prepared by any one of a number of conventional techniques, typically starting from a fluid dispersion. For example, a typical method for preparing drug-containing beads involves dispersing or dissolving the active agent in a coating suspension or solution containing pharmaceutical excipients such as polyvinylpyrrolidone, methylcellulose, talc, metallic stearates, silicone dioxide, plasticizers or the like. The admixture is used to coat a bead core such as a sugar sphere (or so-called “non-pareil”) having a size of approximately 60 to 20 mesh.

An alternative procedure for preparing drug beads is by blending drug with one or more pharmaceutically acceptable excipients, such as microcrystalline cellulose, lactose, cellulose, polyvinyl pyrrolidone, talc, magnesium stearate, a disintegrant, etc., extruding the blend, spheronizing the extrudate, drying and optionally coating to form the immediate release beads.

### 4. Formulations for Mucosal and Pulmonary Administration

The probenecid, metabolites and analogs thereof, and pharmaceutical compositions thereof can be formulated for pulmonary or mucosal administration. The administration can include delivery of the composition to the lungs, nasal, oral (sublingual, buccal), vaginal, or rectal mucosa. In a particular embodiment, the composition is formulated for and delivered to the subject sublingually.

In some embodiments, the compound is formulated for pulmonary delivery, such as intranasal administration or oral inhalation. The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where drug absorption occurs. The alveoli are covered by a thin epithelium without cilia or a mucus blanket and secrete surfactant phospholipids. The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchiole, which then lead to the ultimate respiratory zone, the alveoli, or deep lung. The deep lung, or alveoli, is the primary target of inhaled therapeutic aerosols for systemic drug delivery.

Pulmonary administration of therapeutic compositions comprised of low molecular weight drugs has been observed, for example, beta-androgenic antagonists to treat asthma. Other therapeutic agents that are active in the lungs have been administered systemically and targeted via pulmonary absorption.

Nasal delivery is considered to be a promising technique for administration of therapeutics for the following reasons: the nose has a large surface area available for drug absorption due to the coverage of the epithelial surface by numerous microvilli, the subepithelial layer is highly vascularized, the venous blood from the nose passes directly into the systemic circulation and therefore avoids the loss of drug by first-pass metabolism in the liver, it offers lower doses, more rapid attainment of therapeutic blood levels, quicker onset of pharmacological activity, fewer side effects, high total blood flow per cm³, porous endothelial basement membrane, and it is easily accessible.

The term aerosol as used herein refers to any preparation of a fine mist of particles, which can be in solution or a suspension, whether or not it is produced using a propellant. Aerosols can be produced using standard techniques, such as ultrasonication or high-pressure treatment.

Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into a solution, e.g., water or isotonic saline, buffered or un-buffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2. One skilled in the art can readily determine a suitable saline content and pH for an innocuous aqueous solution for nasal and/or upper respiratory administration.

Preferably, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In another embodiment, solvents that are low toxicity organic (i.e. nonaqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In one embodiment, compositions may contain minor amounts of polymers, surfactants, or other excipients well known to those of the art. In this context, “minor amounts” means no excipients are present that might affect or mediate uptake of the compounds in the lungs and that the excipients that are present are present in amount that do not adversely affect uptake of compounds in the lungs.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+ nebulizer (PARI Respiratory Equipment, Monterey, CA).

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits.

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art. The preferred methods of manufacture are by spray drying and freeze drying, which entails using a solution containing the surfactant, spraying to form droplets of the desired size, and removing the solvent.

The particles may be fabricated with the appropriate material, surface roughness, diameter, and tap density for localized delivery to selected regions of the respiratory tract such as the deep lung or upper airways. For example, higher density or larger particles may be used for upper airway delivery. Similarly, a mixture of different sized particles, provided with the same or different active agents may be administered to target different regions of the lung in one administration.

Compositions and methods of preparing inhalation-type pharmaceutical compositions including probenecid are described in U.S. Published Application No. 2015/0272870.

Thus, formulations and methods of administering the disclosed compositions to the nasal mucosa and/or the lungs by intranasal delivery, and to the lung by oral inhalation are provided. With respect to intranasal delivery, the formulation and delivery device can be selected and prepared to drive absorption through the nasal mucosa or the lungs. The nasal mucosa is—compared to other mucous membranes—easily accessible and provides a practical entrance portal for small and large molecules (Bitter, et al., “Nasal Drug Delivery in Humans,” in Surber, et al., (eds): Topical Applications and the Mucosa. Curr Probl Dermatol. Basel, Karger, 2011, vol 40, pp 20-35, Pires, et al., J Pharm Pharmaceut Sci., 12(3) 288-311, 2009, and Djupesland, Drug Deliv. and Transl. Res., 3:42-62 (2013) DOI 10.1007/s13346-012-0108-9). Intranasal administration offers a rapid onset of therapeutic effects, reduced first—pass effect, reduced gastrointestinal degradation and lung toxicity, noninvasiveness, essentially painless application, and easy and ready use by patients—particularly suited for children—or by physicians in emergency settings. Flu Mist®, for example, is an an exemplary effective nasal influenza vaccine spray.

Numerous delivery devices are available for intranasal administration. Devices vary in accuracy of delivery, dose reproducibility, cost and ease of use. Metered—dose systems provide dose accuracy and reproducibility. Differences also exist in force of delivery, spray patterns and emitted droplet size. The latter being important for drug deposition within the nasal cavity. Parameters can be can be modulated to enhance deposition while limiting the fraction of small particles able to bypass the nose and enter the lungs, or reduce deposition while increasing the fraction of small particles able to bypass the nose and enter the lungs.

The following aspects of nasal anatomy can influence drug delivery. During exhalation the soft palate closes automatically, separating the nasal and oral cavities. Thus, it becomes possible to use smaller particles in a nasal spray and still avoid lung deposition. Additionally, during closure of the soft palate there is a communication pathway between the two nostrils, located behind the walls separating the two passages. Under these circumstances, it is possible for airflow to enter via one nostril and leave by the other. This bidirectional delivery concept combines the two anatomical facts into one fully functional device. The device is inserted into one nostril by a sealing nozzle, and the patient blows into the mouthpiece. The combination of closed soft palate and sealed nozzle creates an airflow which enters one nostril, turns 180° through the communication pathway and exits through the other nostril (bidirectional flow). Since delivery occurs during exhalation, small particles cannot enter the lungs.

Particle size, flow rate and direction can be tuned for efficient delivery to the nasal mucosa. By adding an exit resistor to give additional control of the input pressure, it is possible to improve distribution to the sinuses and the middle ear. Manipulation of the flow pattern enables delivery to the olfactory region, thereby possibly achieving direct ‘nose-to-brain’ delivery. The 180-degree turn behind the septum will trap particles still airborne, allowing targeted delivery of cargo to the adenoid.

Strategies for enhancing drug absorption via nasal and pulmonary routes are also know in the art and can be utilized in the disclosed formulations and methods of delivery. Such strategies include, for example, use of absorption enhancers such as surfactants, cyclodextrins, protease inhibitors, and tight junction modulators, as well as application of carriers such as liposomes and nanoparticles. See, e.g., Ghadiri, et al., Pharmaceutics, 11(3): 113 (2019), which is specifically incorporated by reference herein in its entirety.

### 5. Formulations for Parenteral Administration

Probenecid, metabolites and analogs thereof, and pharmaceutical compositions thereof can be administered in an aqueous solution, by parenteral injection or infusion. The formulation may also be in the form of a suspension or emulsion. In general, pharmaceutical compositions are provided including effective amounts of the active agent(s) and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as POLYSORBATE® 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

### III. Methods of Treatment

Methods of treating a viral infection in subject in need thereof are provided. In some embodiments, the virus can be one that causes a respiratory disease or illness. Thus, methods of treating a respiratory disease or illness, particularly in subject infected with a virus are also provided.

The methods can include administering to a subject an effective amount of probenecid, a metabolite or analog thereof, or a pharmaceutically acceptable salt thereof to reduce viral replication, infection, or a combination thereof.

In some embodiments, the subject has been, or will be, exposed to the virus. In some embodiments, the subject has been exposed to the virus or is experiencing an active viral infection.

The compositions can also be administered prophylactically to, for example, reduce or prevent the effects of future exposure to virus and the infection that may associated therewith. Thus, in some embodiments, the subject has not been exposed to the virus and/or is not yet experiencing an active viral infection. In some embodiments, the subject is a healthy subject.

In some embodiments, the subject will be exposed to the virus. In some embodiments, treatment begins 1, 2, 3, 4, 5, or more hours, days, or weeks prior to or after exposure to the virus.

In some embodiments, the probenecid, a metabolite or analog thereof, or a pharmaceutically acceptable salt thereof is administered in an effective amount to reduce or prevent one or more symptoms of a viral infection. Symptoms include those of an acute respiratory illness, for example, fever, congestion in the nasal sinuses and/or lungs, runny or stuffy nose, cough, sneezing, sore throat, body aches, fatigue, shortness of breath, chest tightness, and wheezing when exhaling. Exemplary viruses and particular symptoms associated with infection thereby are discussed in more detail below. Most typically, the virus is a coronavirus.

In some embodiments, the subject does not have gout, need prolonged penicillin (or other antibiotic) serum levels, pelvic inflammatory disease, or gonorrhea.

In some embodiments, the subject has an influenza infection. See, e.g., Perwitasari, et al., Antimicrob Agents Chemother, 57(1):475-83 (2013). doi: 10.1128/AAC.01532-12.)). For example, in some embodiments, the subject has an influenza (e.g., influenza A, influenza B, influenza C, and/or influenza D) infection and an infection from another virus, such as a coronavirus. In some embodiments, the subject does not have an influenza viral infection.

### A. Exemplary Dosages and Regimens

Probenecid, metabolites and analogs thereof and pharmaceutically acceptable salt thereof can be administered to a subject in a pharmaceutical composition, such as those discussed above, and can be administered by parenteral (intramuscular, intraperitoneal, intravenous (IV) or subcutaneous injection), enteral, transdermal (either passively or using iontophoresis or electroporation), or transmucosal (nasal, pulmonary, vaginal, rectal, or sublingual) routes, as discussed in more detail above.

The precise dosage will vary according to a variety of factors such as subject-dependent variables (e.g., age, immune system health, clinical symptoms, route of delivery, etc.).

For treating gout, probenecid has been administered at 250 mg Per os/oral (PO) twice daily for 1 week; increasing to 500 mg PO twice daily to 2 g/day maximum with dosage increases of 500 mg.

For treating prolong penicillin serum levels, probenecid has been administered at 500 mg PO four times daily.

For pelvic inflammatory disease probenecid has been administered at 1 g PO with 2 g cefoxitin intramuscular (IM) as single dose.

For gonorrhea, probenecid has been administered at 1 g PO with 2 g cefoxitin IM as single dose.

A typically pediatric (e.g., age: 2 to 14 years and weight less than 50 kg) administration as an adjuvant to antibiotic therapy is Initial: 25 mg/kg (or 0.7 g/m2) orally once; Maintenance: 40 mg/kg (or 1.2 g/m2) per day orally administered in 4 equally divided doses 4 times a day.

Thus, in general, by way of example only, dosage forms useful in the disclosed methods may include doses in the range of 0.1 mg to 3,000 mg; 25 mg to 2,000 mg; 25 mg to 1,000 mg; 50 mg to 1,000 mg; 100 mg to 1,000 mg;

or 250 mg to 1,000 mg, with doses of 10 mg, 25 mg, 45 mg, 50 mg, 100 mg, 150 mg, 200 mg, 250 mg, 300 mg, 350 mg, 400 mg, 450 mg, 500 mg, 550 mg, 600 mg, 750 mg, and 1,000 mg being exemplary doses, which can be administered, for example, 1, 2, 3, 4, or 5 times daily, weekly, bi-week, etc., for 1, 2, 3, 4, or more weeks, and for example until symptoms improve or disappear. In some embodiments, a single treatment can be repeated 1, 2, 3, 4, 5, 6, 7, or more days, weeks, or months apart.

In some embodiments, the treatment regimen is similar to those describe above for, e.g., gout, prolonging penicillin serum levels, pelvic inflammatory disease, gonorrhea, etc.

In a particular embodiments, the probenecid or a metabolite or analog thereof or pharmaceutically acceptable salt thereof is administered as 250 mg twice per day.

As introduced above, recitation of ranges of values herein including the dosage ranges above and elsewhere herein, are merely intended to serve as a shorthand method of referring individually to each separate value falling within the range and each separate value is incorporated into the specification as if it were individually recited herein.

Dosing regimens may be, for example, intermittent dosing or continuous (e.g., constant infusion). The dosing regimens can include administrations of the same or different doses. Thus, the dosing regimen can include dose escalation, dose reduction, or a combination thereof.

In some embodiments, the composition is administered in a pulsed dosage regimen. Pulse dosing refers to dosing approach that produces escalating drug levels early in the dosing interval followed by a prolonged dose-free period. For example, in some embodiments, drug administration is frontloaded by means of, for example, 1, 2, 3, 4, or 5 sequential bolus administrations, after which drug levels are allowed to diminish until the next dose. In some embodiments, the serum drug level is allowed to diminish to about 0.

This type of drug delivery technology could offer therapeutic advantages such as reduced dose frequency and greater patient compliance. In comparison to intermittent dosing, pulse dosing front loads the drug, allowing an extended dose-free period during which drug concentration falls close to zero. However, unlike a single, large bolus dose (e.g., given once daily), short bursts of drug are separated by short dose-free periods, allowing the serum concentration to fluctuate (Ibrahim, et al., Antimicrobial Agents and Chemotherapy, 48(11):4195-4199 (2004)). In particular embodiments, pulse dosing is carried out by oral administration or intravenous administration. For example, in some embodiments, the therapy includes discontinuous/intermittent intravenous infusion of very high doses of probenecid, a metabolite or analog thereof, or a pharmaceutically acceptable salt thereof over a short period.

In some embodiments, a large bolus dose of probenecid, a metabolite or analog thereof, or pharmaceutically acceptable salt thereof is between about 1,000 mg and 5,000 mg inclusive, or any subrange or specific dosage there between.

The maximum recommended dosage for probenecid is 2 grams/day PO for adults, adolescents, and children of more than 50 kg, and 40 mg/kg/day (1.2 grams/m2/day) PO (not to exceed 2 grams/day PO) for adolescents and children of 50 kg or less. Thus, in some embodiments, administration does not exceed 5 g, 4 g, 3 g, or 2 g per day. In some embodiments, administration does not exceed 40 mg/kg/day. See also “probenecid—Drug Summary”, the Prescribers' Digital Reference.

In some embodiments, a tablet for oral administration contains e.g., 500 mg of probenecid and optionally, one or more of the following inactive ingredients: microcrystalline cellulose, sodium lauryl sulfate, sodium starch glycolate, starch (corn), povidone, colloidal silicon dioxide, magnesium stearate, polyvinyl alcohol, titanium dioxide, polyethylene glycol, talc, D&C Yellow #10 Aluminum Lake, FD&C Yellow #6 Aluminum Lake, and FD&C Blue #2 Aluminum Lake.

### B. Combination Therapies

In some embodiments, probenecid, a metabolite or analog thereof, or a pharmaceutically acceptable salt thereof is administered in combination with one or more additional active agents. The combination therapies can include administration of the active agents together in the same admixture, or in separate admixtures. Therefore, in some embodiments, the pharmaceutical composition includes two, three, or more active agents. Such formulations typically include an effective amount of probenecid, a metabolite or analog thereof, or a pharmaceutically acceptable salt thereof. In some embodiments, the second active agent is an antiviral (i.e., a second antiviral), a fever reducer, an antinflammatory, an analgesic, or a combination thereof. In a particular embodiment, the antiviral is oseltamivir phosphate (TAMIFLU®). Tamiflu is a prescription medicine used to treat the flu (influenza) in people 2 weeks of age and older who have had flu symptoms. Probenecid or metabolites or analogs or pharmaceutical salts thereof may enhance the efficacy of antivirals like oseltamivir phosphate as it helps retain excretion of the drug during treatment.

### C. Exemplary Viruses and Symptoms

Exemplary viruses and symptoms of illness stemming from infection by the viruses that are treatable by the disclosed methods are also provided. The virus is typically a coronavirus. The current classification of coronaviruses recognizes 39 species in 27 subgenera, five genera and two subfamilies that belong to the family Coronaviridae, suborder Cornidovirineae, order Nidovirales and realm Riboviria (Coronaviridae Study Group of the International Committee on Taxonomy of Viruses, Nat Microbiol 2020. DOI: 10.1038/s41564-020-0695-z). They are enveloped viruses with a positive-sense single-stranded RNA genome and a nucleocapsid of helical symmetry. The genome size of coronaviruses ranges from approximately 26 to 32 kilobases, one of the largest among RNA viruses.

Coronaviruses cause diseases in mammals and birds.

In preferred embodiments, the subject is a human. In humans, coronaviruses can cause respiratory tract infections that can range from mild to lethal. Mild illnesses include some cases of the common cold, while more lethal varieties can cause SARS, MERS, and COVID-19 (i.e., caused by SARS-CoV-2).

The subject may have one or more symptoms characteristic of SARS, MERS, or COVID-19.

SARS (i.e., SAR-CoV) usually begins with flu-like signs and symptoms such as fever, chills, muscle aches, headache and occasionally diarrhea. After about a week, signs and symptoms include fever of 100.5 F (38 C) or higher, dry cough, and shortness of breath.

Reported illnesses from COVID-19 (i.e., caused by SARS-CoV-2) have ranged from mild symptoms to severe illness and death for confirmed cases. The most common symptoms are fever, tiredness, dry cough, and shortness of breath. Runny nose, vomiting and diarrhea have also been reported. These symptoms may appear 2-14 days after exposure.

Most people confirmed to have MERS-CoV infection have had severe respiratory illness with symptoms of fever, cough, and/or shortness of breath. Some people also had diarrhea and nausea/vomiting. For many people with MERS, more severe complications followed, such as pneumonia and kidney failure. Some infected people had mild symptoms (such as cold-like symptoms) or no symptoms at all.

In some embodiments, the subject has an underlying condition such as asthma, heart disease, diabetes, cancer, chronic lung disease, chronic heart disease, chronic kidney disease, or a combination thereof.

In other embodiments, the subject is a non-human mammal or a bird. Symptoms caused by coronavirus infection in non-human species vary: in chickens, they cause an upper respiratory tract disease, while in cows and pigs they cause diarrhea.

Coronavirus species and representative viruses thereof include [representative virus (of species)]: SARSr-CoV BtKY72 (Severe acute respiratory syndrome-related coronavirus), SARS-CoV-2 (Severe acute respiratory syndrome-related coronavirus), SARSr-CoV RaTG13 (Severe acute respiratory syndrome-related coronavirus), SARS-CoV PC4-227 (Severe acute respiratory syndrome-related coronavirus), SARS-CoV (Severe acute respiratory syndrome-related coronavirus), Bat-Hp-BetaCovC (Bat Hp-betacoronavirus Zhejiang2013), Ro-BatCoV GCCDC1 (Rousettus bat coronavirus GCCDC1), Ro-BatCoV HKU9 (Rousettus bat coronavirus HKU9), Ei-BatCoV C704 (Eidolon bat coronavirus C704), Pi-BatCoV HKU5 (Pipistrellus bat coronavirus HKU5), Ty-BatCoV HKU4 (Tylonycteris bar coronovirus HKU4), MERS-CoV (Middle East respiratory syndrome-related coronavirus), EriCoV (Hedgehog coronavirus), MHV (murine coronavirus), HCoV HKU1 (Human coronavirus HKU1), ChRCoV HKU24 (China Rattus coronavirus HKU24), ChRCovC HKU24 (Betacoronavirus 1), MrufCoV 2JL14 (Myodes coronavirus 2JL14), HCoV NL63 (Human coronavirus NL63), HCoV 229E (Human coronavirus 229E), and HCoV OC43 (Human coronavirus OC43). See, e.g., Coronaviridae Study Group of the International Committee on Taxonomy of Viruses, Nat Microbiol 2020. DOI: 10.1038/s41564-020-0695-z), which is specifically incorporated by reference in its entirety. In some embodiments, the coronavirus is a common cold coronavirus such as 229E, NL63, OC43, and HKU1.

In particularly preferred embodiments, the virus is a Severe acute respiratory syndrome-related virus, such as, SARSr-CoV BtKY72, SARS-CoV-2, SARS-CoV RaTG13, SARS-CoV PC4-227, or SARS-CoV, preferably one that infects humans such as SARS-CoV or SARS-CoV-2.

In some embodiments, the virus is a Middle East respiratory syndrome-related virus such as MERS-CoV.

Various strains of the foregoing viruses are known and include the representative genomic sequences provided as, for example, SEQ ID NOS: 1-4, the accession numbers provided herein, and those sequences and accession numbers provided in, e.g., Coronaviridae Study Group of the International Committee on Taxonomy of Viruses, Nat Microbiol 2020. DOI: 10.1038/s41564-020-0695-z). These, however, are non-limiting examples, and the disclosed compositions and methods can also be used to treat other strains of coronavirus, particularly SARS and MERS coronaviruses. In some embodiments, the (DNA sequence) of the viral genome has a sequence at least 80%, preferably at 85%, more preferably at least 86%, 87%, 88%, 89%, 90%, 91%, 92%, 93%, 94%, 95%, 96%, 97%, 98%, 99%, or 100% sequence identity to one or more of SEQ ID NOS:1, 2, 3, or 4, or another viral accession number provided herein, or a sequence or accession number provided in Coronaviridae Study Group of the International Committee on Taxonomy of Viruses, Nat Microbiol 2020. DOI: 10.1038/s41564-020-0695-z, all of which are specifically incorporated by reference herein in their entireties. It will be appreciated that the sequences are provided as DNA sequences, but the viral genome itself will typically have the corresponding RNA sequences. Thus, the corresponding RNA sequences are also expressly provided herein.

GenBank Accession No. MN908947.3, which is specifically incorporated by reference herein in its entirety, provides the following (DNA) genomic sequence for SARS-CoV-2 (Severe acute respiratory syndrome coronavirus 2 isolate Wuhan-Hu-1, complete genome):

| (SEQ ID NO: 1) | |
|---|---|
| 1 | attaaaggtt tataccttcc caggtaacaa accaaccaac tttcgatctc ttgtagatct |
| 61 | gttctctaaa cgaactttaa aatctgtgtg gctgtcactc ggctgcatgc ttagtgcact |
| 121 | cacgcagtat aattaataac taattactgt cgttgacagg acacgagtaa ctcgtctatc |
| 181 | ttctgcaggc tgcttacggt ttcgtccgtg ttgcagccga tcatcagcac atctaggttt |
| 241 | cgtccgggtg tgaccgaaag gtaagatgga gagccttgtc cctggtttca acgagaaaac |
| 301 | acacgtccaa ctcagtttgc ctgttttaca ggttcgcgac gtgctcgtac gtggctttgg |
| 361 | agactccgtg gaggaggtct tatcagaggc acgtcaacat cttaaagatg gcacttgtgg |
| 421 | cttagtagaa gttgaaaaag gcgttttgcc tcaacttgaa cagccctatg tgttcatcaa |
| 481 | acgttcggat gctcgaactg cacctcatgg tcatgttatg gttgagctgg tagcagaact |
| 541 | cgaaggcatt cagtacggtc gtagtggtga gacacttggt gtccttgtcc ctcatgtggg |
| 601 | cgaaatacca gtggcttacc gcaaggttct tcttcgtaag aacggtaata aaggagctgg |
| 661 | tggccatagt tacggcgccg atctaaagtc atttgactta ggcgacgagc ttggcactga |
| 721 | tccttatgaa gattttcaag aaaactggaa cactaaacat agcagtggtg ttacccgtga |
| 781 | actcatgcgt gagcttaacg gaggggcata cactcgctat gtcgataaca acttctgtgg |
| 841 | ccctgatggc taccctcttg agtgcattaa agaccttcta gcacgtgctg gtaaagcttc |
| 901 | atgcactttg tccgaacaac tggactttat tgacactaag aggggtgtat actgctgccg |
| 961 | tgaacatgag catgaaattg cttggtacac ggaacgttct gaaaagagct atgaattgca |
| 1021 | gacacctttt gaaattaaat tggcaaagaa atttgacacc ttcaatgggg aatgtccaaa |
| 1081 | ttttgtattt cccttaaatt ccataatcaa gactattcaa ccaagggttg aaaagaaaaa |
| 1141 | gcttgatggc tttatgggta gaattcgatc tgtctatcca gttgcgtcac caaatgaatg |
| 1201 | caaccaaatg tgcctttcaa ctctcatgaa gtgtgatcat tgtggtgaaa cttcatggca |
| 1261 | gacgggcgat tttgttaaag ccacttgcga attttgtggc actgagaatt tgactaaaga |
| 1321 | aggtgccact acttgtggtt acttacccca aaatgctgtt gttaaaattt attgtccagc |
| 1381 | atgtcacaat tcagaagtag gacctgagca tagtcttgcc gaataccata atgaatctgg |
| 1441 | cttgaaaacc attcttcgta agggtggtcg cactattgcc tttggaggct gtgtgttctc |
| 1501 | ttatgttggt tgccataaca agtgtgccta ttgggttcca cgtgctagcg ctaacatagg |
| 1561 | ttgtaaccat acaggtgttg ttggagaagg ttccgaaggt cttaatgaca accttcttga |
| 1621 | aatactccaa aaagagaaag tcaacatcaa tattgttggt gactttaaac ttaatgaaga |
| 1681 | gatcgccatt attttggcat ctttttctgc ttccacaagt gcttttgtgg aaactgtgaa |
| 1741 | aggtttggat tataaagcat tcaaacaaat tgttgaatcc tgtggtaatt ttaaagttac |
| 1801 | aaaaggaaaa gctaaaaaag gtgcctggaa tattggtgaa cagaaatcaa tactgagtcc |
| 1861 | tctttatgca tttgcatcag aggctgctcg tgttgtacga tcaattttct cccgcactct |
| 1921 | tgaaactgct caaaattctg tgcgtgtttt acagaaggcc gctataacaa tactagatgg |
| 1981 | aatttcacag tattcactga gactcattga tgctatgatg ttcacatctg atttggctac |
| 2041 | taacaatcta gttgtaatgg cctacattac aggtggtgtt gttcagttga cttcgcagtg |
| 2101 | gctaactaac atctttggca ctgtttatga aaaactcaaa cccgtccttg attggcttga |
| 2161 | agagaagttt aaggaaggtg tagagtttct tagagacggt tgggaaattg ttaaatttat |
| 2221 | ctcaacctgt gcttgtgaaa ttgtcggtgg acaaattgtc acctgtgcaa aggaaattaa |
| 2281 | ggagagtgtt cagacattct ttaagcttgt aaataaattt ttggctttgt gtgctgactc |
| 2341 | tatcattatt ggtggagcta aacttaaagc cttgaattta ggtgaaacat ttgtcacgca |
| 2401 | ctcaaaggga ttgtacagaa agtgtgttaa atccagagaa gaaactggcc tactcatgcc |
| 2461 | tctaaaagcc ccaaaagaaa ttatcttctt agagggagaa acacttccca cagaagtgtt |
| 2521 | aacagaggaa gttgtcttga aaactggtga tttacaacca ttagaacaac ctactagtga |
| 2581 | agctgttgaa gctccattgg ttggtacacc agtttgtatt aacgggctta tgttgctcga |
| 2641 | aatcaaagac acagaaaagt actgtgccct tgcacctaat atgatggtaa caaacaatac |
| 2701 | cttcacactc aaaggcggtg caccaacaaa ggttactttt ggtgatgaca ctgtgataga |
| 2761 | agtgcaaggt tacaagagtg tgaatatcac ttttgaactt gatgaaagga ttgataaagt |
| 2821 | acttaatgag aagtgctctg cctatacagt tgaactcggt acagaagtaa atgagttcgc |
| 2881 | ctgtgttgtg gcagatgctg tcataaaaac tttgcaacca gtatctgaat tacttacacc |
| 2941 | actgggcatt gatttagatg agtggagtat ggctacatac tacttatttg atgagtctgg |
| 3001 | tgagtttaaa ttggcttcac atatgtattg ttctttctac cctccagatg aggatgaaga |
| 3061 | agaaggtgat tgtgaagaag aagagtttga gccatcaact caatatgagt atggtactga |
| 3121 | agatgattac caaggtaaac ctttggaatt tggtgccact tctgctgctc ttcaacctga |
| 3181 | agaagagcaa gaagaagatt ggttagatga tgatagtcaa caaactgttg gtcaacaaga |
| 3241 | cggcagtgag gacaatcaga caactactat tcaaacaatt gttgaggttc aacctcaatt |
| 3301 | agagatggaa cttacaccag ttgttcagac tattgaagtg aatagtttta gtggttattt |
| 3361 | aaaacttact gacaatgtat acattaaaaa tgcagacatt gtggaagaag ctaaaaaggt |
| 3421 | aaaaccaaca gtggttgtta atgcagccaa tgtttacctt aaacatggag gaggtgttgc |
| 3481 | aggagcctta aataaggcta ctaacaatgc catgcaagtt gaatctgatg attacatagc |
| 3541 | tactaatgga ccacttaaag tgggtggtag ttgtgtttta agcggacaca atcttgctaa |
| 3601 | acactgtctt catgttgtcg gcccaaatgt taacaaaggt gaagacattc aacttcttaa |
| 3661 | gagtgcttat gaaaatttta atcagcacga agttctactt gcaccattat tatcagctgg |
| 3721 | tatttttggt gctgacccta tacattcttt aagagtttgt gtagatactg ttcgcacaaa |
| 3781 | tgtctactta gctgtctttg ataaaaatct ctatgacaaa cttgtttcaa gctttttgga |
| 3841 | aatgaagagt gaaaagcaag ttgaacaaaa gatcgctgag attcctaaag aggaagttaa |
| 3901 | gccatttata actgaaagta aaccttcagt tgaacagaga aaacaagatg ataagaaaat |
| 3961 | caaagcttgt gttgaagaag ttacaacaac tctggaagaa actaagttcc tcacagaaaa |
| 4021 | cttgttactt tatattgaca ttaatggcaa tcttcatcca gattctgcca ctcttgttag |
| 4081 | tgacattgac atcactttct taaagaaaga tgctccatat atagtgggtg atgttgttca |
| 4141 | agagggtgtt ttaactgctg tggttatacc tactaaaaag gctggtggca ctactgaaat |
| 4201 | gctagcgaaa gctttgagaa aagtgccaac agacaattat ataaccactt acccgggtca |
| 4261 | gggtttaaat ggttacactg tagaggaggc aaagacagtg cttaaaaagt gtaaaagtgc |
| 4321 | cttttacatt ctaccatcta ttatctctaa tgagaagcaa gaaattcttg gaactgtttc |
| 4381 | ttggaatttg cgagaaatgc ttgcacatgc agaagaaaca cgcaaattaa tgcctgtctg |
| 4441 | tgtggaaact aaagccatag tttcaactat acagcgtaaa tataagggta ttaaaataca |
| 4501 | agagggtgtg gttgattatg gtgctagatt ttacttttac accagtaaaa caactgtagc |
| 4561 | gtcacttatc aacacactta acgatctaaa tgaaactctt gttacaatgc cacttggcta |
| 4621 | tgtaacacat ggcttaaatt tggaagaagc tgctcggtat atgagatctc tcaaagtgcc |
| 4681 | agctacagtt tctgtttctt cacctgatgc tgttacagcg tataatggtt atcttacttc |
| 4741 | ttcttctaaa acacctgaag aacattttat tgaaaccatc tcacttgctg gttcctataa |
| 4801 | agattggtcc tattctggac aatctacaca actaggtata gaatttctta agagaggtga |
| 4861 | taaaagtgta tattacacta gtaatcctac cacattccac ctagatggtg aagttatcac |
| 4921 | ctttgacaat cttaagacac ttctttcttt gagagaagtg aggactatta aggtgtttac |
| 4981 | aacagtagac aacattaacc tccacacgca agttgtggac atgtcaatga catatggaca |
| 5041 | acagtttggt ccaacttatt tggatggagc tgatgttact aaaataaaac ctcataattc |
| 5101 | acatgaaggt aaaacatttt atgttttacc taatgatgac actctacgtg ttgaggcttt |
| 5161 | tgagtactac cacacaactg atcctagttt tctgggtagg tacatgtcag cattaaatca |
| 5221 | cactaaaaag tggaaatacc cacaagttaa tggtttaact tctattaaat gggcagataa |
| 5281 | caactgttat cttgccactg cattgttaac actccaacaa atagagttga agtttaatcc |
| 5341 | acctgctcta caagatgctt attacagagc aagggctggt gaagctgcta acttttgtgc |
| 5401 | acttatctta gcctactgta ataagacagt aggtgagtta ggtgatgtta gagaaacaat |
| 5461 | gagttacttg tttcaacatg ccaatttaga ttcttgcaaa agagtcttga acgtggtgtg |
| 5521 | taaaacttgt ggacaacagc agacaaccct taagggtgta gaagctgtta tgtacatggg |
| 5581 | cacactttct tatgaacaat ttaagaaagg tgttcagata ccttgtacgt gtggtaaaca |
| 5641 | agctacaaaa tatctagtac aacaggagtc accttttgtt atgatgtcag caccacctgc |
| 5701 | tcagtatgaa cttaagcatg gtacatttac ttgtgctagt gagtacactg gtaattacca |
| 5761 | gtgtggtcac tataaacata taacttctaa agaaactttg tattgcatag acggtgcttt |
| 5821 | acttacaaag tcctcagaat acaaaggtcc tattacggat gttttctaca aagaaaacag |
| 5881 | ttacacaaca accataaaac cagttactta taaattggat ggtgttgttt gtacagaaat |
| 5941 | tgaccctaag ttggacaatt attataagaa agacaattct tatttcacag agcaaccaat |
| 6001 | tgatcttgta ccaaaccaac catatccaaa cgcaagcttc gataatttta agtttgtatg |
| 6061 | tgataatatc aaatttgctg atgatttaaa ccagttaact ggttataaga aacctgcttc |
| 6121 | aagagagctt aaagttacat ttttccctga cttaaatggt gatgtggtgg ctattgatta |
| 6181 | taaacactac acaccctctt ttaagaaagg agctaaattg ttacataaac ctattgtttg |
| 6241 | gcatgttaac aatgcaacta ataaagccac gtataaacca aatacctggt gtatacgttg |
| 6301 | tctttggagc acaaaaccag ttgaaacatc aaattcgttt gatgtactga agtcagagga |
| 6361 | cgcgcaggga atggataatc ttgcctgcga agatctaaaa ccagtctctg aagaagtagt |
| 6421 | ggaaaatcct accatacaga aagacgttct tgagtgtaat gtgaaaacta ccgaagttgt |
| 6481 | aggagacatt atacttaaac cagcaaataa tagtttaaaa attacagaag aggttggcca |
| 6541 | cacagatcta atggctgctt atgtagacaa ttctagtctt actattaaga aacctaatga |
| 6601 | attatctaga gtattaggtt tgaaaaccct tgctactcat ggtttagctg ctgttaatag |
| 6661 | tgtcccttgg gatactatag ctaattatgc taagcctttt cttaacaaag ttgttagtac |
| 6721 | aactactaac atagttacac ggtgtttaaa ccgtgtttgt actaattata tgccttattt |
| 6781 | ctttacttta ttgctacaat tgtgtacttt tactagaagt acaaattcta gaattaaagc |
| 6841 | atctatgccg actactatag caaagaatac tgttaagagt gtcggtaaat tttgtctaga |
| 6901 | ggcttcattt aattatttga agtcacctaa tttttctaaa ctgataaata ttataatttg |
| 6961 | gtttttacta ttaagtgttt gcctaggttc tttaatctac tcaaccgctg ctttaggtgt |
| 7021 | tttaatgtct aatttaggca tgccttctta ctgtactggt tacagagaag gctatttgaa |
| 7081 | ctctactaat gtcactattg caacctactg tactggttct ataccttgta gtgtttgtct |
| 7141 | tagtggttta gattctttag acacctatcc ttctttagaa actatacaaa ttaccatttc |
| 7201 | atcttttaaa tgggatttaa ctgcttttgg cttagttgca gagtggtttt tggcatatat |
| 7261 | tcttttcact aggtttttct atgtacttgg attggctgca atcatgcaat tgtttttcag |
| 7321 | ctattttgca gtacatttta ttagtaattc ttggcttatg tggttaataa ttaatcttgt |
| 7381 | acaaatggcc ccgatttcag ctatggttag aatgtacatc ttctttgcat cattttatta |
| 7441 | tgtatggaaa agttatgtgc atgttgtaga cggttgtaat tcatcaactt gtatgatgtg |
| 7501 | ttacaaacgt aatagagcaa caagagtcga atgtacaact attgttaatg gtgttagaag |
| 7561 | gtccttttat gtctatgcta atggaggtaa aggcttttgc aaactacaca attggaattg |
| 7621 | tgttaattgt gatacattct gtgctggtag tacatttatt agtgatgaag ttgcgagaga |
| 7681 | cttgtcacta cagtttaaaa gaccaataaa tcctactgac cagtcttctt acatcgttga |
| 7741 | tagtgttaca gtgaagaatg gttccatcca tctttacttt gataaagctg gtcaaaagac |
| 7801 | ttatgaaaga cattctctct ctcattttgt taacttagac aacctgagag ctaataacac |
| 7861 | taaaggttca ttgcctatta atgttatagt ttttgatggt aaatcaaaat gtgaagaatc |
| 7921 | atctgcaaaa tcagcgtctg tttactacag tcagcttatg tgtcaaccta tactgttact |
| 7981 | agatcaggca ttagtgtctg atgttggtga tagtgcggaa gttgcagtta aaatgtttga |
| 8041 | tgcttacgtt aatacgtttt catcaacttt taacgtacca atggaaaaac tcaaaacact |
| 8101 | agttgcaact gcagaagctg aacttgcaaa gaatgtgtcc ttagacaatg tcttatctac |
| 8161 | ttttatttca gcagctcggc aagggtttgt tgattcagat gtagaaacta aagatgttgt |
| 8221 | tgaatgtctt aaattgtcac atcaatctga catagaagtt actggcgata gttgtaataa |
| 8281 | ctatatgctc acctataaca aagttgaaaa catgacaccc cgtgaccttg gtgcttgtat |
| 8341 | tgactgtagt gcgcgtcata ttaatgcgca ggtagcaaaa agtcacaaca ttgctttgat |
| 8401 | atggaacgtt aaagatttca tgtcattgtc tgaacaacta cgaaaacaaa tacgtagtgc |
| 8461 | tgctaaaaag aataacttac cttttaagtt gacatgtgca actactagac aagttgttaa |
| 8521 | tgttgtaaca acaaagatag cacttaaggg tggtaaaatt gttaataatt ggttgaagca |
| 8581 | gttaattaaa gttacacttg tgttcctttt tgttgctgct attttctatt taataacacc |
| 8641 | tgttcatgtc atgtctaaac atactgactt ttcaagtgaa atcataggat acaaggctat |
| 8701 | tgatggtggt gtcactcgtg acatagcatc tacagatact tgttttgcta acaaacatgc |
| 8761 | tgattttgac acatggttta gccagcgtgg tggtagttat actaatgaca aagcttgccc |
| 8821 | attgattgct gcagtcataa caagagaagt gggttttgtc gtgcctggtt tgcctggcac |
| 8881 | gatattacgc acaactaatg gtgacttttt gcatttctta cctagagttt ttagtgcagt |
| 8941 | tggtaacatc tgttacacac catcaaaact tatagagtac actgactttg caacatcagc |
| 9001 | ttgtgttttg gctgctgaat gtacaatttt taaagatgct tctggtaagc cagtaccata |
| 9061 | ttgttatgat accaatgtac tagaaggttc tgttgcttat gaaagtttac gccctgacac |
| 9121 | acgttatgtg ctcatggatg gctctattat tcaatttcct aacacctacc ttgaaggttc |
| 9181 | tgttagagtg gtaacaactt ttgattctga gtactgtagg cacggcactt gtgaaagatc |
| 9241 | agaagctggt gtttgtgtat ctactagtgg tagatgggta cttaacaatg attattacag |
| 9301 | atctttacca ggagttttct gtggtgtaga tgctgtaaat ttacttacta atatgtttac |
| 9361 | accactaatt caacctattg gtgctttgga catatcagca tctatagtag ctggtggtat |
| 9421 | tgtagctatc gtagtaacat gccttgccta ctattttatg aggtttagaa gagcttttgg |
| 9481 | tgaatacagt catgtagttg cctttaatac tttactattc cttatgtcat tcactgtact |
| 9541 | ctgtttaaca ccagtttact cattcttacc tggtgtttat tctgttattt acttgtactt |
| 9601 | gacattttat cttactaatg atgtttcttt tttagcacat attcagtgga tggttatgtt |
| 9661 | cacaccttta gtacctttct ggataacaat tgcttatatc atttgtattt ccacaaagca |
| 9721 | tttctattgg ttctttagta attacctaaa gagacgtgta gtctttaatg gtgtttcctt |
| 9781 | tagtactttt gaagaagctg cgctgtgcac ctttttgtta aataaagaaa tgtatctaaa |
| 9841 | gttgcgtagt gatgtgctat tacctcttac gcaatataat agatacttag ctctttataa |
| 9901 | taagtacaag tattttagtg gagcaatgga tacaactagc tacagagaag ctgcttgttg |
| 9961 | tcatctcgca aaggctctca atgacttcag taactcaggt tctgatgttc tttaccaacc |
| 10021 | accacaaacc tctatcacct cagctgtttt gcagagtggt tttagaaaaa tggcattccc |
| 10081 | atctggtaaa gttgagggtt gtatggtaca agtaacttgt ggtacaacta cacttaacgg |
| 10141 | tctttggctt gatgacgtag tttactgtcc aagacatgtg atctgcacct ctgaagacat |
| 10201 | gcttaaccct aattatgaag atttactcat tcgtaagtct aatcataatt tcttggtaca |
| 10261 | ggctggtaat gttcaactca gggttattgg acattctatg caaaattgtg tacttaagct |
| 10321 | taaggttgat acagccaatc ctaagacacc taagtataag tttgttcgca ttcaaccagg |
| 10381 | acagactttt tcagtgttag cttgttacaa tggttcacca tctggtgttt accaatgtgc |
| 10441 | tatgaggccc aatttcacta ttaagggttc attccttaat ggttcatgtg gtagtgttgg |
| 10501 | ttttaacata gattatgact gtgtctcttt ttgttacatg caccatatgg aattaccaac |
| 10561 | tggagttcat gctggcacag acttagaagg taacttttat ggaccttttg ttgacaggca |
| 10621 | aacagcacaa gcagctggta cggacacaac tattacagtt aatgttttag cttggttgta |
| 10681 | cgctgctgtt ataaatggag acaggtggtt tctcaatcga tttaccacaa ctcttaatga |
| 10741 | ctttaacctt gtggctatga agtacaatta tgaacctcta acacaagacc atgttgacat |
| 10801 | actaggacct ctttctgctc aaactggaat tgccgtttta gatatgtgtg cttcattaaa |
| 10861 | agaattactg caaaatggta tgaatggacg taccatattg ggtagtgctt tattagaaga |
| 10921 | tgaatttaca ccttttgatg ttgttagaca atgctcaggt gttactttcc aaagtgcagt |
| 10981 | gaaaagaaca atcaagggta cacaccactg gttgttactc acaattttga cttcactttt |
| 11041 | agttttagtc cagagtactc aatggtcttt gttctttttt ttgtatgaaa atgccttttt |
| 11101 | accttttgct atgggtatta ttgctatgtc tgcttttgca atgatgtttg tcaaacataa |
| 11161 | gcatgcattt ctctgtttgt ttttgttacc ttctcttgcc actgtagctt attttaatat |
| 11221 | ggtctatatg cctgctagtt gggtgatgcg tattatgaca tggttggata tggttgatac |
| 11281 | tagtttgtct ggttttaagc taaaagactg tgttatgtat gcatcagctg tagtgttact |
| 11341 | aatccttatg acagcaagaa ctgtgtatga tgatggtgct aggagagtgt ggacacttat |
| 11401 | gaatgtcttg acactcgttt ataaagttta ttatggtaat gctttagatc aagccatttc |
| 11461 | catgtgggct cttataatct ctgttacttc taactactca ggtgtagtta caactgtcat |
| 11521 | gtttttggcc agaggtattg tttttatgtg tgttgagtat tgccctattt tcttcataac |
| 11581 | tggtaataca cttcagtgta taatgctagt ttattgtttc ttaggctatt tttgtacttg |
| 11641 | ttactttggc ctcttttgtt tactcaaccg ctactttaga ctgactcttg gtgtttatga |
| 11701 | ttacttagtt tctacacagg agtttagata tatgaattca cagggactac tcccacccaa |
| 11761 | gaatagcata gatgccttca aactcaacat taaattgttg ggtgttggtg gcaaaccttg |
| 11821 | tatcaaagta gccactgtac agtctaaaat gtcagatgta aagtgcacat cagtagtctt |
| 11881 | actctcagtt ttgcaacaac tcagagtaga atcatcatct aaattgtggg ctcaatgtgt |
| 11941 | ccagttacac aatgacattc tcttagctaa agatactact gaagcctttg aaaaaatggt |
| 12001 | ttcactactt tctgttttgc tttccatgca gggtgctgta gacataaaca agctttgtga |
| 12061 | agaaatgctg gacaacaggg caaccttaca agctatagcc tcagagttta gttcccttcc |
| 12121 | atcatatgca gcttttgcta ctgctcaaga agcttatgag caggctgttg ctaatggtga |
| 12181 | ttctgaagtt gttcttaaaa agttgaagaa gtctttgaat gtggctaaat ctgaatttga |
| 12241 | ccgtgatgca gccatgcaac gtaagttgga aaagatggct gatcaagcta tgacccaaat |
| 12301 | gtataaacag gctagatctg aggacaagag ggcaaaagtt actagtgcta tgcagacaat |
| 12361 | gcttttcact atgcttagaa agttggataa tgatgcactc aacaacatta tcaacaatgc |
| 12421 | aagagatggt tgtgttccct tgaacataat acctcttaca acagcagcca aactaatggt |
| 12481 | tgtcatacca gactataaca catataaaaa tacgtgtgat ggtacaacat ttacttatgc |
| 12541 | atcagcattg tgggaaatcc aacaggttgt agatgcagat agtaaaattg ttcaacttag |
| 12601 | tgaaattagt atggacaatt cacctaattt agcatggcct cttattgtaa cagctttaag |
| 12661 | ggccaattct gctgtcaaat tacagaataa tgagcttagt cctgttgcac tacgacagat |
| 12721 | gtcttgtgct gccggtacta cacaaactgc ttgcactgat gacaatgcgt tagcttacta |
| 12781 | caacacaaca aagggaggta ggtttgtact tgcactgtta tccgatttac aggatttgaa |
| 12841 | atgggctaga ttccctaaga gtgatggaac tggtactatc tatacagaac tggaaccacc |
| 12901 | ttgtaggttt gttacagaca cacctaaagg tcctaaagtg aagtatttat actttattaa |
| 12961 | aggattaaac aacctaaata gaggtatggt acttggtagt ttagctgcca cagtacgtct |
| 13021 | acaagctggt aatgcaacag aagtgcctgc caattcaact gtattatctt tctgtgcttt |
| 13081 | tgctgtagat gctgctaaag cttacaaaga ttatctagct agtgggggac aaccaatcac |
| 13141 | taattgtgtt aagatgttgt gtacacacac tggtactggt caggcaataa cagttacacc |
| 13201 | ggaagccaat atggatcaag aatcctttgg tggtgcatcg tgttgtctgt actgccgttg |
| 13261 | ccacatagat catccaaatc ctaaaggatt ttgtgactta aaaggtaagt atgtacaaat |
| 13321 | acctacaact tgtgctaatg accctgtggg ttttacactt aaaaacacag tctgtaccgt |
| 13381 | ctgcggtatg tggaaaggtt atggctgtag ttgtgatcaa ctccgcgaac ccatgcttca |
| 13441 | gtcagctgat gcacaatcgt ttttaaacgg gtttgcggtg taagtgcagc ccgtcttaca |
| 13501 | ccgtgcggca caggcactag tactgatgtc gtatacaggg cttttgacat ctacaatgat |
| 13561 | aaagtagctg gttttgctaa attcctaaaa actaattgtt gtcgcttcca agaaaaggac |
| 13621 | gaagatgaca atttaattga ttcttacttt gtagttaaga gacacacttt ctctaactac |
| 13681 | caacatgaag aaacaattta taatttactt aaggattgtc cagctgttgc taaacatgac |
| 13741 | ttctttaagt ttagaataga cggtgacatg gtaccacata tatcacgtca acgtcttact |
| 13801 | aaatacacaa tggcagacct cgtctatgct ttaaggcatt ttgatgaagg taattgtgac |
| 13861 | acattaaaag aaatacttgt cacatacaat tgttgtgatg atgattattt caataaaaag |
| 13921 | gactggtatg attttgtaga aaacccagat atattacgcg tatacgccaa cttaggtgaa |
| 13981 | cgtgtacgcc aagctttgtt aaaaacagta caattctgtg atgccatgcg aaatgctggt |
| 14041 | attgttggtg tactgacatt agataatcaa gatctcaatg gtaactggta tgatttcggt |
| 14101 | gatttcatac aaaccacgcc aggtagtgga gttcctgttg tagattctta ttattcattg |
| 14161 | ttaatgccta tattaacctt gaccagggct ttaactgcag agtcacatgt tgacactgac |
| 14221 | ttaacaaagc cttacattaa gtgggatttg ttaaaatatg acttcacgga agagaggtta |
| 14281 | aaactctttg accgttattt taaatattgg gatcagacat accacccaaa ttgtgttaac |
| 14341 | tgtttggatg acagatgcat tctgcattgt gcaaacttta atgttttatt ctctacagtg |
| 14401 | ttcccaccta caagttttgg accactagtg agaaaaatat ttgttgatgg tgttccattt |
| 14461 | gtagtttcaa ctggatacca cttcagagag ctaggtgttg tacataatca ggatgtaaac |
| 14521 | ttacatagct ctagacttag ttttaaggaa ttacttgtgt atgctgctga ccctgctatg |
| 14581 | cacgctgctt ctggtaatct attactagat aaacgcacta cgtgcttttc agtagctgca |
| 14641 | cttactaaca atgttgcttt tcaaactgtc aaacccggta attttaacaa agacttctat |
| 14701 | gactttgctg tgtctaaggg tttctttaag gaaggaagtt ctgttgaatt aaaacacttc |
| 14761 | ttctttgctc aggatggtaa tgctgctatc agcgattatg actactatcg ttataatcta |
| 14821 | ccaacaatgt gtgatatcag acaactacta tttgtagttg aagttgttga taagtacttt |
| 14881 | gattgttacg atggtggctg tattaatgct aaccaagtca tcgtcaacaa cctagacaaa |
| 14941 | tcagctggtt ttccatttaa taaatggggt aaggctagac tttattatga ttcaatgagt |
| 15001 | tatgaggatc aagatgcact tttcgcatat acaaaacgta atgtcatccc tactataact |
| 15061 | caaatgaatc ttaagtatgc cattagtgca aagaatagag ctcgcaccgt agctggtgtc |
| 15121 | tctatctgta gtactatgac caatagacag tttcatcaaa aattattgaa atcaatagcc |
| 15181 | gccactagag gagctactgt agtaattgga acaagcaaat tctatggtgg ttggcacaac |
| 15241 | atgttaaaaa ctgtttatag tgatgtagaa aaccctcacc ttatgggttg ggattatcct |
| 15301 | aaatgtgata gagccatgcc taacatgctt agaattatgg cctcacttgt tcttgctcgc |
| 15361 | aaacatacaa cgtgttgtag cttgtcacac cgtttctata gattagctaa tgagtgtgct |
| 15421 | caagtattga gtgaaatggt catgtgtggc ggttcactat atgttaaacc aggtggaacc |
| 15481 | tcatcaggag atgccacaac tgcttatgct aatagtgttt ttaacatttg tcaagctgtc |
| 15541 | acggccaatg ttaatgcact tttatctact gatggtaaca aaattgccga taagtatgtc |
| 15601 | cgcaatttac aacacagact ttatgagtgt ctctatagaa atagagatgt tgacacagac |
| 15661 | tttgtgaatg agttttacgc atatttgcgt aaacatttct caatgatgat actctctgac |
| 15721 | gatgctgttg tgtgtttcaa tagcacttat gcatctcaag gtctagtggc tagcataaag |
| 15781 | aactttaagt cagttcttta ttatcaaaac aatgttttta tgtctgaagc aaaatgttgg |
| 15841 | actgagactg accttactaa aggacctcat gaattttgct ctcaacatac aatgctagtt |
| 15901 | aaacagggtg atgattatgt gtaccttcct tacccagatc catcaagaat cctaggggcc |
| 15961 | ggctgttttg tagatgatat cgtaaaaaca gatggtacac ttatgattga acggttcgtg |
| 16021 | tctttagcta tagatgctta cccacttact aaacatccta atcaggagta tgctgatgtc |
| 16081 | tttcatttgt acttacaata cataagaaag ctacatgatg agttaacagg acacatgtta |
| 16141 | gacatgtatt ctgttatgct tactaatgat aacacttcaa ggtattggga acctgagttt |
| 16201 | tatgaggcta tgtacacacc gcatacagtc ttacaggctg ttggggcttg tgttctttgc |
| 16261 | aattcacaga cttcattaag atgtggtgct tgcatacgta gaccattctt atgttgtaaa |
| 16321 | tgctgttacg accatgtcat atcaacatca cataaattag tcttgtctgt taatccgtat |
| 16381 | gtttgcaatg ctccaggttg tgatgtcaca gatgtgactc aactttactt aggaggtatg |
| 16441 | agctattatt gtaaatcaca taaaccaccc attagttttc cattgtgtgc taatggacaa |
| 16501 | gtttttggtt tatataaaaa tacatgtgtt ggtagcgata atgttactga ctttaatgca |
| 16561 | attgcaacat gtgactggac aaatgctggt gattacattt tagctaacac ctgtactgaa |
| 16621 | agactcaagc tttttgcagc agaaacgctc aaagctactg aggagacatt taaactgtct |
| 16681 | tatggtattg ctactgtacg tgaagtgctg tctgacagag aattacatct ttcatgggaa |
| 16741 | gttggtaaac ctagaccacc acttaaccga aattatgtct ttactggtta tcgtgtaact |
| 16801 | aaaaacagta aagtacaaat aggagagtac acctttgaaa aaggtgacta tggtgatgct |
| 16861 | gttgtttacc gaggtacaac aacttacaaa ttaaatgttg gtgattattt tgtgctgaca |
| 16921 | tcacatacag taatgccatt aagtgcacct acactagtgc cacaagagca ctatgttaga |
| 16981 | attactggct tatacccaac actcaatatc tcagatgagt tttctagcaa tgttgcaaat |
| 17041 | tatcaaaagg ttggtatgca aaagtattct acactccagg gaccacctgg tactggtaag |
| 17101 | agtcattttg ctattggcct agctctctac tacccttctg ctcgcatagt gtatacagct |
| 17161 | tgctctcatg ccgctgttga tgcactatgt gagaaggcat taaaatattt gcctatagat |
| 17221 | aaatgtagta gaattatacc tgcacgtgct cgtgtagagt gttttgataa attcaaagtg |
| 17281 | aattcaacat tagaacagta tgtcttttgt actgtaaatg cattgcctga gacgacagca |
| 17341 | gatatagttg tctttgatga aatttcaatg gccacaaatt atgatttgag tgttgtcaat |
| 17401 | gccagattac gtgctaagca ctatgtgtac attggcgacc ctgctcaatt acctgcacca |
| 17461 | cgcacattgc taactaaggg cacactagaa ccagaatatt tcaattcagt gtgtagactt |
| 17521 | atgaaaacta taggtccaga catgttcctc ggaacttgtc ggcgttgtcc tgctgaaatt |
| 17581 | gttgacactg tgagtgcttt ggtttatgat aataagctta aagcacataa agacaaatca |
| 17641 | gctcaatgct ttaaaatgtt ttataagggt gttatcacgc atgatgtttc atctgcaatt |
| 17701 | aacaggccac aaataggcgt ggtaagagaa ttccttacac gtaaccctgc ttggagaaaa |
| 17761 | gctgtcttta tttcacctta taattcacag aatgctgtag cctcaaagat tttgggacta |
| 17821 | ccaactcaaa ctgttgattc atcacagggc tcagaatatg actatgtcat attcactcaa |
| 17881 | accactgaaa cagctcactc ttgtaatgta aacagattta atgttgctat taccagagca |
| 17941 | aaagtaggca tactttgcat aatgtctgat agagaccttt atgacaagtt gcaatttaca |
| 18001 | agtcttgaaa ttccacgtag gaatgtggca actttacaag ctgaaaatgt aacaggactc |
| 18061 | tttaaagatt gtagtaaggt aatcactggg ttacatccta cacaggcacc tacacacctc |
| 18121 | agtgttgaca ctaaattcaa aactgaaggt ttatgtgttg acatacctgg catacctaag |
| 18181 | gacatgacct atagaagact catctctatg atgggtttta aaatgaatta tcaagttaat |
| 18241 | ggttacccta acatgtttat cacccgcgaa gaagctataa gacatgtacg tgcatggatt |
| 18301 | ggcttcgatg tcgaggggtg tcatgctact agagaagctg ttggtaccaa tttaccttta |
| 18361 | cagctaggtt tttctacagg tgttaaccta gttgctgtac ctacaggtta tgttgataca |
| 18421 | cctaataata cagatttttc cagagttagt gctaaaccac cgcctggaga tcaatttaaa |
| 18481 | cacctcatac cacttatgta caaaggactt ccttggaatg tagtgcgtat aaagattgta |
| 18541 | caaatgttaa gtgacacact taaaaatctc tctgacagag tcgtatttgt cttatgggca |
| 18601 | catggctttg agttgacatc tatgaagtat tttgtgaaaa taggacctga gcgcacctgt |
| 18661 | tgtctatgtg atagacgtgc cacatgcttt tccactgctt cagacactta tgcctgttgg |
| 18721 | catcattcta ttggatttga ttacgtctat aatccgttta tgattgatgt tcaacaatgg |
| 18781 | ggttttacag gtaacctaca aagcaaccat gatctgtatt gtcaagtcca tggtaatgca |
| 18841 | catgtagcta gttgtgatgc aatcatgact aggtgtctag ctgtccacga gtgctttgtt |
| 18901 | aagcgtgttg actggactat tgaatatcct ataattggtg atgaactgaa gattaatgcg |
| 18961 | gcttgtagaa aggttcaaca catggttgtt aaagctgcat tattagcaga caaattccca |
| 19021 | gttcttcacg acattggtaa ccctaaagct attaagtgtg tacctcaagc tgatgtagaa |
| 19081 | tggaagttct atgatgcaca gccttgtagt gacaaagctt ataaaataga agaattattc |
| 19141 | tattcttatg ccacacattc tgacaaattc acagatggtg tatgcctatt ttggaattgc |
| 19201 | aatgtcgata gatatcctgc taattccatt gtttgtagat ttgacactag agtgctatct |
| 19261 | aaccttaact tgcctggttg tgatggtggc agtttgtatg taaataaaca tgcattccac |
| 19321 | acaccagctt ttgataaaag tgcttttgtt aatttaaaac aattaccatt tttctattac |
| 19381 | tctgacagtc catgtgagtc tcatggaaaa caagtagtgt cagatataga ttatgtacca |
| 19441 | ctaaagtctg ctacgtgtat aacacgttgc aatttaggtg gtgctgtctg tagacatcat |
| 19501 | gctaatgagt acagattgta tctcgatgct tataacatga tgatctcagc tggctttagc |
| 19561 | ttgtgggttt acaaacaatt tgatacttat aacctctgga acacttttac aagacttcag |
| 19621 | agtttagaaa atgtggcttt taatgttgta aataagggac actttgatgg acaacagggt |
| 19681 | gaagtaccag tttctatcat taataacact gtttacacaa aagttgatgg tgttgatgta |
| 19741 | gaattgtttg aaaataaaac aacattacct gttaatgtag catttgagct ttgggctaag |
| 19801 | cgcaacatta aaccagtacc agaggtgaaa atactcaata atttgggtgt ggacattgct |
| 19861 | gctaatactg tgatctggga ctacaaaaga gatgctccag cacatatatc tactattggt |
| 19921 | gtttgttcta tgactgacat agccaagaaa ccaactgaaa cgatttgtgc accactcact |
| 19981 | gtcttttttg atggtagagt tgatggtcaa gtagacttat ttagaaatgc ccgtaatggt |
| 20041 | gttcttatta cagaaggtag tgttaaaggt ttacaaccat ctgtaggtcc caaacaagct |
| 20101 | agtcttaatg gagtcacatt aattggagaa gccgtaaaaa cacagttcaa ttattataag |
| 20161 | aaagttgatg gtgttgtcca acaattacct gaaacttact ttactcagag tagaaattta |
| 20221 | caagaattta aacccaggag tcaaatggaa attgatttct tagaattagc tatggatgaa |
| 20281 | ttcattgaac ggtataaatt agaaggctat gccttcgaac atatcgttta tggagatttt |
| 20341 | agtcatagtc agttaggtgg tttacatcta ctgattggac tagctaaacg ttttaaggaa |
| 20401 | tcaccttttg aattagaaga ttttattcct atggacagta cagttaaaaa ctatttcata |
| 20461 | acagatgcgc aaacaggttc atctaagtgt gtgtgttctg ttattgattt attacttgat |
| 20521 | gattttgttg aaataataaa atcccaagat ttatctgtag tttctaaggt tgtcaaagtg |
| 20581 | actattgact atacagaaat ttcatttatg ctttggtgta aagatggcca tgtagaaaca |
| 20641 | ttttacccaa aattacaatc tagtcaagcg tggcaaccgg gtgttgctat gcctaatctt |
| 20701 | tacaaaatgc aaagaatgct attagaaaag tgtgaccttc aaaattatgg tgatagtgca |
| 20761 | acattaccta aaggcataat gatgaatgtc gcaaaatata ctcaactgtg tcaatattta |
| 20821 | aacacattaa cattagctgt accctataat atgagagtta tacattttgg tgctggttct |
| 20881 | gataaaggag ttgcaccagg tacagctgtt ttaagacagt ggttgcctac gggtacgctg |
| 20941 | cttgtcgatt cagatcttaa tgactttgtc tctgatgcag attcaacttt gattggtgat |
| 21001 | tgtgcaactg tacatacagc taataaatgg gatctcatta ttagtgatat gtacgaccct |
| 21061 | aagactaaaa atgttacaaa agaaaatgac tctaaagagg gttttttcac ttacatttgt |
| 21121 | gggtttatac aacaaaagct agctcttgga ggttccgtgg ctataaagat aacagaacat |
| 21181 | tcttggaatg ctgatcttta taagctcatg ggacacttcg catggtggac agcctttgtt |
| 21241 | actaatgtga atgcgtcatc atctgaagca tttttaattg gatgtaatta tcttggcaaa |
| 21301 | ccacgcgaac aaatagatgg ttatgtcatg catgcaaatt acatattttg gaggaataca |
| 21361 | aatccaattc agttgtcttc ctattcttta tttgacatga gtaaatttcc ccttaaatta |
| 21421 | aggggtactg ctgttatgtc tttaaaagaa ggtcaaatca atgatatgat tttatctctt |
| 21481 | cttagtaaag gtagacttat aattagagaa aacaacagag ttgttatttc tagtgatgtt |
| 21541 | cttgttaaca actaaacgaa caatgtttgt ttttcttgtt ttattgccac tagtctctag |
| 21601 | tcagtgtgtt aatcttacaa ccagaactca attaccccct gcatacacta attctttcac |
| 21661 | acgtggtgtt tattaccctg acaaagtttt cagatcctca gttttacatt caactcagga |
| 21721 | cttgttctta cctttctttt ccaatgttac ttggttccat gctatacatg tctctgggac |
| 21781 | caatggtact aagaggtttg ataaccctgt cctaccattt aatgatggtg tttattttgc |
| 21841 | ttccactgag aagtctaaca taataagagg ctggattttt ggtactactt tagattcgaa |
| 21901 | gacccagtcc ctacttattg ttaataacgc tactaatgtt gttattaaag tctgtgaatt |
| 21961 | tcaattttgt aatgatccat ttttgggtgt ttattaccac aaaaacaaca aaagttggat |
| 22021 | ggaaagtgag ttcagagttt attctagtgc gaataattgc acttttgaat atgtctctca |
| 22081 | gccttttctt atggaccttg aaggaaaaca gggtaatttc aaaaatctta gggaatttgt |
| 22141 | gtttaagaat attgatggtt attttaaaat atattctaag cacacgccta ttaatttagt |
| 22201 | gcgtgatctc cctcagggtt tttcggcttt agaaccattg gtagatttgc caataggtat |
| 22261 | taacatcact aggtttcaaa ctttacttgc tttacataga agttatttga ctcctggtga |
| 22321 | ttcttcttca ggttggacag ctggtgctgc agcttattat gtgggttatc ttcaacctag |
| 22381 | gacttttcta ttaaaatata atgaaaatgg aaccattaca gatgctgtag actgtgcact |
| 22441 | tgaccctctc tcagaaacaa agtgtacgtt gaaatccttc actgtagaaa aaggaatcta |
| 22501 | tcaaacttct aactttagag tccaaccaac agaatctatt gttagatttc ctaatattac |
| 22561 | aaacttgtgc ccttttggtg aagtttttaa cgccaccaga tttgcatctg tttatgcttg |
| 22621 | gaacaggaag agaatcagca actgtgttgc tgattattct gtcctatata attccgcatc |
| 22681 | attttccact tttaagtgtt atggagtgtc tcctactaaa ttaaatgatc tctgctttac |
| 22741 | taatgtctat gcagattcat ttgtaattag aggtgatgaa gtcagacaaa tcgctccagg |
| 22801 | gcaaactgga aagattgctg attataatta taaattacca gatgatttta caggctgcgt |
| 22861 | tatagcttgg aattctaaca atcttgattc taaggttggt ggtaattata attacctgta |
| 22921 | tagattgttt aggaagtcta atctcaaacc ttttgagaga gatatttcaa ctgaaatcta |
| 22981 | tcaggccggt agcacacctt gtaatggtgt tgaaggtttt aattgttact ttcctttaca |
| 23041 | atcatatggt ttccaaccca ctaatggtgt tggttaccaa ccatacagag tagtagtact |
| 23101 | ttcttttgaa cttctacatg caccagcaac tgtttgtgga cctaaaaagt ctactaattt |
| 23161 | ggttaaaaac aaatgtgtca atttcaactt caatggttta acaggcacag gtgttcttac |
| 23221 | tgagtctaac aaaaagtttc tgcctttcca acaatttggc agagacattg ctgacactac |
| 23281 | tgatgctgtc cgtgatccac agacacttga gattcttgac attacaccat gttcttttgg |
| 23341 | tggtgtcagt gttataacac caggaacaaa tacttctaac caggttgctg ttctttatca |
| 23401 | ggatgttaac tgcacagaag tccctgttgc tattcatgca gatcaactta ctcctacttg |
| 23461 | gcgtgtttat tctacaggtt ctaatgtttt tcaaacacgt gcaggctgtt taataggggc |
| 23521 | tgaacatgtc aacaactcat atgagtgtga catacccatt ggtgcaggta tatgcgctag |
| 23581 | ttatcagact cagactaatt ctcctcggcg ggcacgtagt gtagctagtc aatccatcat |
| 23641 | tgcctacact atgtcacttg gtgcagaaaa ttcagttgct tactctaata actctattgc |
| 23701 | catacccaca aattttacta ttagtgttac cacagaaatt ctaccagtgt ctatgaccaa |
| 23761 | gacatcagta gattgtacaa tgtacatttg tggtgattca actgaatgca gcaatctttt |
| 23821 | gttgcaatat ggcagttttt gtacacaatt aaaccgtgct ttaactggaa tagctgttga |
| 23881 | acaagacaaa aacacccaag aagtttttgc acaagtcaaa caaatttaca aaacaccacc |
| 23941 | aattaaagat tttggtggtt ttaatttttc acaaatatta ccagatccat caaaaccaag |
| 24001 | caagaggtca tttattgaag atctactttt caacaaagtg acacttgcag atgctggctt |
| 24061 | catcaaacaa tatggtgatt gccttggtga tattgctgct agagacctca tttgtgcaca |
| 24121 | aaagtttaac ggccttactg ttttgccacc tttgctcaca gatgaaatga ttgctcaata |
| 24181 | cacttctgca ctgttagcgg gtacaatcac ttctggttgg acctttggtg caggtgctgc |
| 24241 | attacaaata ccatttgcta tgcaaatggc ttataggttt aatggtattg gagttacaca |
| 24301 | gaatgttctc tatgagaacc aaaaattgat tgccaaccaa tttaatagtg ctattggcaa |
| 24361 | aattcaagac tcactttctt ccacagcaag tgcacttgga aaacttcaag atgtggtcaa |
| 24421 | ccaaaatgca caagctttaa acacgcttgt taaacaactt agctccaatt ttggtgcaat |
| 24481 | ttcaagtgtt ttaaatgata tcctttcacg tcttgacaaa gttgaggctg aagtgcaaat |
| 24541 | tgataggttg atcacaggca gacttcaaag tttgcagaca tatgtgactc aacaattaat |
| 24601 | tagagctgca gaaatcagag cttctgctaa tcttgctgct actaaaatgt cagagtgtgt |
| 24661 | acttggacaa tcaaaaagag ttgatttttg tggaaagggc tatcatctta tgtccttccc |
| 24721 | tcagtcagca cctcatggtg tagtcttctt gcatgtgact tatgtccctg cacaagaaaa |
| 24781 | gaacttcaca actgctcctg ccatttgtca tgatggaaaa gcacactttc ctcgtgaagg |
| 24841 | tgtctttgtt tcaaatggca cacactggtt tgtaacacaa aggaattttt atgaaccaca |
| 24901 | aatcattact acagacaaca catttgtgtc tggtaactgt gatgttgtaa taggaattgt |
| 24961 | caacaacaca gtttatgatc ctttgcaacc tgaattagac tcattcaagg aggagttaga |
| 25021 | taaatatttt aagaatcata catcaccaga tgttgattta ggtgacatct ctggcattaa |
| 25081 | tgcttcagtt gtaaacattc aaaaagaaat tgaccgcctc aatgaggttg ccaagaattt |
| 25141 | aaatgaatct ctcatcgatc tccaagaact tggaaagtat gagcagtata taaaatggcc |
| 25201 | atggtacatt tggctaggtt ttatagctgg cttgattgcc atagtaatgg tgacaattat |
| 25261 | gctttgctgt atgaccagtt gctgtagttg tctcaagggc tgttgttctt gtggatcctg |
| 25321 | ctgcaaattt gatgaagacg actctgagcc agtgctcaaa ggagtcaaat tacattacac |
| 25381 | ataaacgaac ttatggattt gtttatgaga atcttcacaa ttggaactgt aactttgaag |
| 25441 | caaggtgaaa tcaaggatgc tactccttca gattttgttc gcgctactgc aacgataccg |
| 25501 | atacaagcct cactcccttt cggatggctt attgttggcg ttgcacttct tgctgttttt |
| 25561 | cagagcgctt ccaaaatcat aaccctcaaa aagagatggc aactagcact ctccaagggt |
| 25621 | gttcactttg tttgcaactt gctgttgttg tttgtaacag tttactcaca ccttttgctc |
| 25681 | gttgctgctg gccttgaagc cccttttctc tatctttatg ctttagtcta cttcttgcag |
| 25741 | agtataaact ttgtaagaat aataatgagg ctttggcttt gctggaaatg ccgttccaaa |
| 25801 | aacccattac tttatgatgc caactatttt ctttgctggc atactaattg ttacgactat |
| 25861 | tgtatacctt acaatagtgt aacttcttca attgtcatta cttcaggtga tggcacaaca |
| 25921 | agtcctattt ctgaacatga ctaccagatt ggtggttata ctgaaaaatg ggaatctgga |
| 25981 | gtaaaagact gtgttgtatt acacagttac ttcacttcag actattacca gctgtactca |
| 26041 | actcaattga gtacagacac tggtgttgaa catgttacct tcttcatcta caataaaatt |
| 26101 | gttgatgagc ctgaagaaca tgtccaaatt cacacaatcg acggttcatc cggagttgtt |
| 26161 | aatccagtaa tggaaccaat ttatgatgaa ccgacgacga ctactagcgt gcctttgtaa |
| 26221 | gcacaagctg atgagtacga acttatgtac tcattcgttt cggaagagac aggtacgtta |
| 26281 | atagttaata gcgtacttct ttttcttgct ttcgtggtat tcttgctagt tacactagcc |
| 26341 | atccttactg cgcttcgatt gtgtgcgtac tgctgcaata ttgttaacgt gagtcttgta |
| 26401 | aaaccttctt tttacgttta ctctcgtgtt aaaaatctga attcttctag agttcctgat |
| 26461 | cttctggtct aaacgaacta aatattatat tagtttttct gtttggaact ttaattttag |
| 26521 | ccatggcaga ttccaacggt actattaccg ttgaagagct taaaaagctc cttgaacaat |
| 26581 | ggaacctagt aataggtttc ctattcctta catggatttg tcttctacaa tttgcctatg |
| 26641 | ccaacaggaa taggtttttg tatataatta agttaatttt cctctggctg ttatggccag |
| 26701 | taactttagc ttgttttgtg cttgctgctg tttacagaat aaattggatc accggtggaa |
| 26761 | ttgctatcgc aatggcttgt cttgtaggct tgatgtggct cagctacttc attgcttctt |
| 26821 | tcagactgtt tgcgcgtacg cgttccatgt ggtcattcaa tccagaaact aacattcttc |
| 26881 | tcaacgtgcc actccatggc actattctga ccagaccgct tctagaaagt gaactcgtaa |
| 26941 | tcggagctgt gatccttcgt ggacatcttc gtattgctgg acaccatcta ggacgctgtg |
| 27001 | acatcaagga cctgcctaaa gaaatcactg ttgctacatc acgaacgctt tcttattaca |
| 27061 | aattgggagc ttcgcagcgt gtagcaggtg actcaggttt tgctgcatac agtcgctaca |
| 27121 | ggattggcaa ctataaatta aacacagacc attccagtag cagtgacaat attgctttgc |
| 27181 | ttgtacagta agtgacaaca gatgtttcat ctcgttgact ttcaggttac tatagcagag |
| 27241 | atattactaa ttattatgag gacttttaaa gtttccattt ggaatcttga ttacatcata |
| 27301 | aacctcataa ttaaaaattt atctaagtca ctaactgaga ataaatattc tcaattagat |
| 27361 | gaagagcaac caatggagat tgattaaacg aacatgaaaa ttattctttt cttggcactg |
| 27421 | ataacactcg ctacttgtga gctttatcac taccaagagt gtgttagagg tacaacagta |
| 27481 | cttttaaaag aaccttgctc ttctggaaca tacgagggca attcaccatt tcatcctcta |
| 27541 | gctgataaca aatttgcact gacttgcttt agcactcaat ttgcttttgc ttgtcctgac |
| 27601 | ggcgtaaaac acgtctatca gttacgtgcc agatcagttt cacctaaact gttcatcaga |
| 27661 | caagaggaag ttcaagaact ttactctcca atttttctta ttgttgcggc aatagtgttt |
| 27721 | ataacacttt gcttcacact caaaagaaag acagaatgat tgaactttca ttaattgact |
| 27781 | tctatttgtg ctttttagcc tttctgctat tccttgtttt aattatgctt attatctttt |
| 27841 | ggttctcact tgaactgcaa gatcataatg aaacttgtca cgcctaaacg aacatgaaat |
| 27901 | ttcttgtttt cttaggaatc atcacaactg tagctgcatt tcaccaagaa tgtagtttac |
| 27961 | agtcatgtac tcaacatcaa ccatatgtag ttgatgaccc gtgtcctatt cacttctatt |
| 28021 | ctaaatggta tattagagta ggagctagaa aatcagcacc tttaattgaa ttgtgcgtgg |
| 28081 | atgaggctgg ttctaaatca cccattcagt acatcgatat cggtaattat acagtttcct |
| 28141 | gtttaccttt tacaattaat tgccaggaac ctaaattggg tagtcttgta gtgcgttgtt |
| 28201 | cgttctatga agacttttta gagtatcatg acgttcgtgt tgttttagat ttcatctaaa |
| 28261 | cgaacaaact aaaatgtctg ataatggacc ccaaaatcag cgaaatgcac cccgcattac |
| 28321 | gtttggtgga ccctcagatt caactggcag taaccagaat ggagaacgca gtggggcgcg |
| 28381 | atcaaaacaa cgtcggcccc aaggtttacc caataatact gcgtcttggt tcaccgctct |
| 28441 | cactcaacat ggcaaggaag accttaaatt ccctcgagga caaggcgttc caattaacac |
| 28501 | caatagcagt ccagatgacc aaattggcta ctaccgaaga gctaccagac gaattcgtgg |
| 28561 | tggtgacggt aaaatgaaag atctcagtcc aagatggtat ttctactacc taggaactgg |
| 28621 | gccagaagct ggacttccct atggtgctaa caaagacggc atcatatggg ttgcaactga |
| 28681 | gggagccttg aatacaccaa aagatcacat tggcacccgc aatcctgcta acaatgctgc |
| 28741 | aatcgtgcta caacttcctc aaggaacaac attgccaaaa ggcttctacg cagaagggag |
| 28801 | cagaggcggc agtcaagcct cttctcgttc ctcatcacgt agtcgcaaca gttcaagaaa |
| 28861 | ttcaactcca ggcagcagta ggggaacttc tcctgctaga atggctggca atggcggtga |
| 28921 | tgctgctctt gctttgctgc tgcttgacag attgaaccag cttgagagca aaatgtctgg |
| 28981 | taaaggccaa caacaacaag gccaaactgt cactaagaaa tctgctgctg aggcttctaa |
| 29041 | gaagcctcgg caaaaacgta ctgccactaa agcatacaat gtaacacaag ctttcggcag |
| 29101 | acgtggtcca gaacaaaccc aaggaaattt tggggaccag gaactaatca gacaaggaac |
| 29161 | tgattacaaa cattggccgc aaattgcaca atttgccccc agcgcttcag cgttcttcgg |
| 29221 | aatgtcgcgc attggcatgg aagtcacacc ttcgggaacg tggttgacct acacaggtgc |
| 29281 | catcaaattg gatgacaaag atccaaattt caaagatcaa gtcattttgc tgaataagca |
| 29341 | tattgacgca tacaaaacat tcccaccaac agagcctaaa aaggacaaaa agaagaaggc |
| 29401 | tgatgaaact caagccttac cgcagagaca gaagaaacag caaactgtga ctcttcttcc |
| 29461 | tgctgcagat ttggatgatt tctccaaaca attgcaacaa tccatgagca gtgctgactc |
| 29521 | aactcaggcc taaactcatg cagaccacac aaggcagatg ggctatataa acgttttcgc |
| 29581 | ttttccgttt acgatatata gtctactctt gtgcagaatg aattctcgta actacatagc |
| 29641 | acaagtagat gtagttaact ttaatctcac atagcaatct ttaatcagtg tgtaacatta |
| 29701 | gggaggactt gaaagagcca ccacattttc accgaggcca cgcggagtac gatcgagtgt |
| 29761 | acagtgaaca atgctaggga gagctgccta tatggaagag ccctaatgtg taaaattaat |
| 29821 | tttagtagtg ctatccccat gtgattttaa tagcttctta ggagaatgac aaaaaaaaaa |
| 29881 | aaaaaaaaaa aaaaaaaaaa aaa. |

GenBank Accession No. MN985325.1. which is specifically incorporated by reference herein in its entirety, provides the following (DNA) genomic sequence for SARS-CoV-2 (Severe acute respiratory syndrome coronavirus 2 isolate 2019-nCoV/USA-WA1/2020, complete genome):

| (SEQ ID NO: 2) | |
|---|---|
| 1 | attaaaggtt tataccttcc caggtaacaa accaaccaac tttcgatctc ttgtagatct |
| 61 | gttctctaaa cgaactttaa aatctgtgtg gctgtcactc ggctgcatgc ttagtgcact |
| 121 | cacgcagtat aattaataac taattactgt cgttgacagg acacgagtaa ctcgtctatc |
| 181 | ttctgcaggc tgcttacggt ttcgtccgtg ttgcagccga tcatcagcac atctaggttt |
| 241 | cgtccgggtg tgaccgaaag gtaagatgga gagccttgtc cctggtttca acgagaaaac |
| 301 | acacgtccaa ctcagtttgc ctgttttaca ggttcgcgac gtgctcgtac gtggctttgg |
| 361 | agactccgtg gaggaggtct tatcagaggc acgtcaacat cttaaagatg gcacttgtgg |
| 421 | cttagtagaa gttgaaaaag gcgttttgcc tcaacttgaa cagccctatg tgttcatcaa |
| 481 | acgttcggat gctcgaactg cacctcatgg tcatgttatg gttgagctgg tagcagaact |
| 541 | cgaaggcatt cagtacggtc gtagtggtga gacacttggt gtccttgtcc ctcatgtggg |
| 601 | cgaaatacca gtggcttacc gcaaggttct tcttcgtaag aacggtaata aaggagctgg |
| 661 | tggccatagt tacggcgccg atctaaagtc atttgactta ggcgacgagc ttggcactga |
| 721 | tccttatgaa gattttcaag aaaactggaa cactaaacat agcagtggtg ttacccgtga |
| 781 | actcatgcgt gagcttaacg gaggggcata cactcgctat gtcgataaca acttctgtgg |
| 841 | ccctgatggc taccctcttg agtgcattaa agaccttcta gcacgtgctg gtaaagcttc |
| 901 | atgcactttg tccgaacaac tggactttat tgacactaag aggggtgtat actgctgccg |
| 961 | tgaacatgag catgaaattg cttggtacac ggaacgttct gaaaagagct atgaattgca |
| 1021 | gacacctttt gaaattaaat tggcaaagaa atttgacacc ttcaatgggg aatgtccaaa |
| 1081 | ttttgtattt cccttaaatt ccataatcaa gactattcaa ccaagggttg aaaagaaaaa |
| 1141 | gcttgatggc tttatgggta gaattcgatc tgtctatcca gttgcgtcac caaatgaatg |
| 1201 | caaccaaatg tgcctttcaa ctctcatgaa gtgtgatcat tgtggtgaaa cttcatggca |
| 1261 | gacgggcgat tttgttaaag ccacttgcga attttgtggc actgagaatt tgactaaaga |
| 1321 | aggtgccact acttgtggtt acttacccca aaatgctgtt gttaaaattt attgtccagc |
| 1381 | atgtcacaat tcagaagtag gacctgagca tagtcttgcc gaataccata atgaatctgg |
| 1441 | cttgaaaacc attcttcgta agggtggtcg cactattgcc tttggaggct gtgtgttctc |
| 1501 | ttatgttggt tgccataaca agtgtgccta ttgggttcca cgtgctagcg ctaacatagg |
| 1561 | ttgtaaccat acaggtgttg ttggagaagg ttccgaaggt cttaatgaca accttcttga |
| 1621 | aatactccaa aaagagaaag tcaacatcaa tattgttggt gactttaaac ttaatgaaga |
| 1681 | gatcgccatt attttggcat ctttttctgc ttccacaagt gcttttgtgg aaactgtgaa |
| 1741 | aggtttggat tataaagcat tcaaacaaat tgttgaatcc tgtggtaatt ttaaagttac |
| 1801 | aaaaggaaaa gctaaaaaag gtgcctggaa tattggtgaa cagaaatcaa tactgagtcc |
| 1861 | tctttatgca tttgcatcag aggctgctcg tgttgtacga tcaattttct cccgcactct |
| 1921 | tgaaactgct caaaattctg tgcgtgtttt acagaaggcc gctataacaa tactagatgg |
| 1981 | aatttcacag tattcactga gactcattga tgctatgatg ttcacatctg atttggctac |
| 2041 | taacaatcta gttgtaatgg cctacattac aggtggtgtt gttcagttga cttcgcagtg |
| 2101 | gctaactaac atctttggca ctgtttatga aaaactcaaa cccgtccttg attggcttga |
| 2161 | agagaagttt aaggaaggtg tagagtttct tagagacggt tgggaaattg ttaaatttat |
| 2221 | ctcaacctgt gcttgtgaaa ttgtcggtgg acaaattgtc acctgtgcaa aggaaattaa |
| 2281 | ggagagtgtt cagacattct ttaagcttgt aaataaattt ttggctttgt gtgctgactc |
| 2341 | tatcattatt ggtggagcta aacttaaagc cttgaattta ggtgaaacat ttgtcacgca |
| 2401 | ctcaaaggga ttgtacagaa agtgtgttaa atccagagaa gaaactggcc tactcatgcc |
| 2461 | tctaaaagcc ccaaaagaaa ttatcttctt agagggagaa acacttccca cagaagtgtt |
| 2521 | aacagaggaa gttgtcttga aaactggtga tttacaacca ttagaacaac ctactagtga |
| 2581 | agctgttgaa gctccattgg ttggtacacc agtttgtatt aacgggctta tgttgctcga |
| 2641 | aatcaaagac acagaaaagt actgtgccct tgcacctaat atgatggtaa caaacaatac |
| 2701 | cttcacactc aaaggcggtg caccaacaaa ggttactttt ggtgatgaca ctgtgataga |
| 2761 | agtgcaaggt tacaagagtg tgaatatcac ttttgaactt gatgaaagga ttgataaagt |
| 2821 | acttaatgag aagtgctctg cctatacagt tgaactcggt acagaagtaa atgagttcgc |
| 2881 | ctgtgttgtg gcagatgctg tcataaaaac tttgcaacca gtatctgaat tacttacacc |
| 2941 | actgggcatt gatttagatg agtggagtat ggctacatac tacttatttg atgagtctgg |
| 3001 | tgagtttaaa ttggcttcac atatgtattg ttctttctac cctccagatg aggatgaaga |
| 3061 | agaaggtgat tgtgaagaag aagagtttga gccatcaact caatatgagt atggtactga |
| 3121 | agatgattac caaggtaaac ctttggaatt tggtgccact tctgctgctc ttcaacctga |
| 3181 | agaagagcaa gaagaagatt ggttagatga tgatagtcaa caaactgttg gtcaacaaga |
| 3241 | cggcagtgag gacaatcaga caactactat tcaaacaatt gttgaggttc aacctcaatt |
| 3301 | agagatggaa cttacaccag ttgttcagac tattgaagtg aatagtttta gtggttattt |
| 3361 | aaaacttact gacaatgtat acattaaaaa tgcagacatt gtggaagaag ctaaaaaggt |
| 3421 | aaaaccaaca gtggttgtta atgcagccaa tgtttacctt aaacatggag gaggtgttgc |
| 3481 | aggagcctta aataaggcta ctaacaatgc catgcaagtt gaatctgatg attacatagc |
| 3541 | tactaatgga ccacttaaag tgggtggtag ttgtgtttta agcggacaca atcttgctaa |
| 3601 | acactgtctt catgttgtcg gcccaaatgt taacaaaggt gaagacattc aacttcttaa |
| 3661 | gagtgcttat gaaaatttta atcagcacga agttctactt gcaccattat tatcagctgg |
| 3721 | tatttttggt gctgacccta tacattcttt aagagtttgt gtagatactg ttcgcacaaa |
| 3781 | tgtctactta gctgtctttg ataaaaatct ctatgacaaa cttgtttcaa gctttttgga |
| 3841 | aatgaagagt gaaaagcaag ttgaacaaaa gatcgctgag attcctaaag aggaagttaa |
| 3901 | gccatttata actgaaagta aaccttcagt tgaacagaga aaacaagatg ataagaaaat |
| 3961 | caaagcttgt gttgaagaag ttacaacaac tctggaagaa actaagttcc tcacagaaaa |
| 4021 | cttgttactt tatattgaca ttaatggcaa tcttcatcca gattctgcca ctcttgttag |
| 4081 | tgacattgac atcactttct taaagaaaga tgctccatat atagtgggtg atgttgttca |
| 4141 | agagggtgtt ttaactgctg tggttatacc tactaaaaag gctggtggca ctactgaaat |
| 4201 | gctagcgaaa gctttgagaa aagtgccaac agacaattat ataaccactt acccgggtca |
| 4261 | gggtttaaat ggttacactg tagaggaggc aaagacagtg cttaaaaagt gtaaaagtgc |
| 4321 | cttttacatt ctaccatcta ttatctctaa tgagaagcaa gaaattcttg gaactgtttc |
| 4381 | ttggaatttg cgagaaatgc ttgcacatgc agaagaaaca cgcaaattaa tgcctgtctg |
| 4441 | tgtggaaact aaagccatag tttcaactat acagcgtaaa tataagggta ttaaaataca |
| 4501 | agagggtgtg gttgattatg gtgctagatt ttacttttac accagtaaaa caactgtagc |
| 4561 | gtcacttatc aacacactta acgatctaaa tgaaactctt gttacaatgc cacttggcta |
| 4621 | tgtaacacat ggcttaaatt tggaagaagc tgctcggtat atgagatctc tcaaagtgcc |
| 4681 | agctacagtt tctgtttctt cacctgatgc tgttacagcg tataatggtt atcttacttc |
| 4741 | ttcttctaaa acacctgaag aacattttat tgaaaccatc tcacttgctg gttcctataa |
| 4801 | agattggtcc tattctggac aatctacaca actaggtata gaatttctta agagaggtga |
| 4861 | taaaagtgta tattacacta gtaatcctac cacattccac ctagatggtg aagttatcac |
| 4921 | ctttgacaat cttaagacac ttctttcttt gagagaagtg aggactatta aggtgtttac |
| 4981 | aacagtagac aacattaacc tccacacgca agttgtggac atgtcaatga catatggaca |
| 5041 | acagtttggt ccaacttatt tggatggagc tgatgttact aaaataaaac ctcataattc |
| 5101 | acatgaaggt aaaacatttt atgttttacc taatgatgac actctacgtg ttgaggcttt |
| 5161 | tgagtactac cacacaactg atcctagttt tctgggtagg tacatgtcag cattaaatca |
| 5221 | cactaaaaag tggaaatacc cacaagttaa tggtttaact tctattaaat gggcagataa |
| 5281 | caactgttat cttgccactg cattgttaac actccaacaa atagagttga agtttaatcc |
| 5341 | acctgctcta caagatgctt attacagagc aagggctggt gaagctgcta acttttgtgc |
| 5401 | acttatctta gcctactgta ataagacagt aggtgagtta ggtgatgtta gagaaacaat |
| 5461 | gagttacttg tttcaacatg ccaatttaga ttcttgcaaa agagtcttga acgtggtgtg |
| 5521 | taaaacttgt ggacaacagc agacaaccct taagggtgta gaagctgtta tgtacatggg |
| 5581 | cacactttct tatgaacaat ttaagaaagg tgttcagata ccttgtacgt gtggtaaaca |
| 5641 | agctacaaaa tatctagtac aacaggagtc accttttgtt atgatgtcag caccacctgc |
| 5701 | tcagtatgaa cttaagcatg gtacatttac ttgtgctagt gagtacactg gtaattacca |
| 5761 | gtgtggtcac tataaacata taacttctaa agaaactttg tattgcatag acggtgcttt |
| 5821 | acttacaaag tcctcagaat acaaaggtcc tattacggat gttttctaca aagaaaacag |
| 5881 | ttacacaaca accataaaac cagttactta taaattggat ggtgttgttt gtacagaaat |
| 5941 | tgaccctaag ttggacaatt attataagaa agacaattct tatttcacag agcaaccaat |
| 6001 | tgatcttgta ccaaaccaac catatccaaa cgcaagcttc gataatttta agtttgtatg |
| 6061 | tgataatatc aaatttgctg atgatttaaa ccagttaact ggttataaga aacctgcttc |
| 6121 | aagagagctt aaagttacat ttttccctga cttaaatggt gatgtggtgg ctattgatta |
| 6181 | taaacactac acaccctctt ttaagaaagg agctaaattg ttacataaac ctattgtttg |
| 6241 | gcatgttaac aatgcaacta ataaagccac gtataaacca aatacctggt gtatacgttg |
| 6301 | tctttggagc acaaaaccag ttgaaacatc aaattcgttt gatgtactga agtcagagga |
| 6361 | cgcgcaggga atggataatc ttgcctgcga agatctaaaa ccagtctctg aagaagtagt |
| 6421 | ggaaaatcct accatacaga aagacgttct tgagtgtaat gtgaaaacta ccgaagttgt |
| 6481 | aggagacatt atacttaaac cagcaaataa tagtttaaaa attacagaag aggttggcca |
| 6541 | cacagatcta atggctgctt atgtagacaa ttctagtctt actattaaga aacctaatga |
| 6601 | attatctaga gtattaggtt tgaaaaccct tgctactcat ggtttagctg ctgttaatag |
| 6661 | tgtcccttgg gatactatag ctaattatgc taagcctttt cttaacaaag ttgttagtac |
| 6721 | aactactaac atagttacac ggtgtttaaa ccgtgtttgt actaattata tgccttattt |
| 6781 | ctttacttta ttgctacaat tgtgtacttt tactagaagt acaaattcta gaattaaagc |
| 6841 | atctatgccg actactatag caaagaatac tgttaagagt gtcggtaaat tttgtctaga |
| 6901 | ggcttcattt aattatttga agtcacctaa tttttctaaa ctgataaata ttataatttg |
| 6961 | gtttttacta ttaagtgttt gcctaggttc tttaatctac tcaaccgctg ctttaggtgt |
| 7021 | tttaatgtct aatttaggca tgccttctta ctgtactggt tacagagaag gctatttgaa |
| 7081 | ctctactaat gtcactattg caacctactg tactggttct ataccttgta gtgtttgtct |
| 7141 | tagtggttta gattctttag acacctatcc ttctttagaa actatacaaa ttaccatttc |
| 7201 | atcttttaaa tgggatttaa ctgcttttgg cttagttgca gagtggtttt tggcatatat |
| 7261 | tcttttcact aggtttttct atgtacttgg attggctgca atcatgcaat tgtttttcag |
| 7321 | ctattttgca gtacatttta ttagtaattc ttggcttatg tggttaataa ttaatcttgt |
| 7381 | acaaatggcc ccgatttcag ctatggttag aatgtacatc ttctttgcat cattttatta |
| 7441 | tgtatggaaa agttatgtgc atgttgtaga cggttgtaat tcatcaactt gtatgatgtg |
| 7501 | ttacaaacgt aatagagcaa caagagtcga atgtacaact attgttaatg gtgttagaag |
| 7561 | gtccttttat gtctatgcta atggaggtaa aggcttttgc aaactacaca attggaattg |
| 7621 | tgttaattgt gatacattct gtgctggtag tacatttatt agtgatgaag ttgcgagaga |
| 7681 | cttgtcacta cagtttaaaa gaccaataaa tcctactgac cagtcttctt acatcgttga |
| 7741 | tagtgttaca gtgaagaatg gttccatcca tctttacttt gataaagctg gtcaaaagac |
| 7801 | ttatgaaaga cattctctct ctcattttgt taacttagac aacctgagag ctaataacac |
| 7861 | taaaggttca ttgcctatta atgttatagt ttttgatggt aaatcaaaat gtgaagaatc |
| 7921 | atctgcaaaa tcagcgtctg tttactacag tcagcttatg tgtcaaccta tactgttact |
| 7981 | agatcaggca ttagtgtctg atgttggtga tagtgcggaa gttgcagtta aaatgtttga |
| 8041 | tgcttacgtt aatacgtttt catcaacttt taacgtacca atggaaaaac tcaaaacact |
| 8101 | agttgcaact gcagaagctg aacttgcaaa gaatgtgtcc ttagacaatg tcttatctac |
| 8161 | ttttatttca gcagctcggc aagggtttgt tgattcagat gtagaaacta aagatgttgt |
| 8221 | tgaatgtctt aaattgtcac atcaatctga catagaagtt actggcgata gttgtaataa |
| 8281 | ctatatgctc acctataaca aagttgaaaa catgacaccc cgtgaccttg gtgcttgtat |
| 8341 | tgactgtagt gcgcgtcata ttaatgcgca ggtagcaaaa agtcacaaca ttgctttgat |
| 8401 | atggaacgtt aaagatttca tgtcattgtc tgaacaacta cgaaaacaaa tacgtagtgc |
| 8461 | tgctaaaaag aataacttac cttttaagtt gacatgtgca actactagac aagttgttaa |
| 8521 | tgttgtaaca acaaagatag cacttaaggg tggtaaaatt gttaataatt ggttgaagca |
| 8581 | gttaattaaa gttacacttg tgttcctttt tgttgctgct attttctatt taataacacc |
| 8641 | tgttcatgtc atgtctaaac atactgactt ttcaagtgaa atcataggat acaaggctat |
| 8701 | tgatggtggt gtcactcgtg acatagcatc tacagatact tgttttgcta acaaacatgc |
| 8761 | tgattttgac acatggttta gtcagcgtgg tggtagttat actaatgaca aagcttgccc |
| 8821 | attgattgct gcagtcataa caagagaagt gggttttgtc gtgcctggtt tgcctggcac |
| 8881 | gatattacgc acaactaatg gtgacttttt gcatttctta cctagagttt ttagtgcagt |
| 8941 | tggtaacatc tgttacacac catcaaaact tatagagtac actgactttg caacatcagc |
| 9001 | ttgtgttttg gctgctgaat gtacaatttt taaagatgct tctggtaagc cagtaccata |
| 9061 | ttgttatgat accaatgtac tagaaggttc tgttgcttat gaaagtttac gccctgacac |
| 9121 | acgttatgtg ctcatggatg gctctattat tcaatttcct aacacctacc ttgaaggttc |
| 9181 | tgttagagtg gtaacaactt ttgattctga gtactgtagg cacggcactt gtgaaagatc |
| 9241 | agaagctggt gtttgtgtat ctactagtgg tagatgggta cttaacaatg attattacag |
| 9301 | atctttacca ggagttttct gtggtgtaga tgctgtaaat ttacttacta atatgtttac |
| 9361 | accactaatt caacctattg gtgctttgga catatcagca tctatagtag ctggtggtat |
| 9421 | tgtagctatc gtagtaacat gccttgccta ctattttatg aggtttagaa gagcttttgg |
| 9481 | tgaatacagt catgtagttg cctttaatac tttactattc cttatgtcat tcactgtact |
| 9541 | ctgtttaaca ccagtttact cattcttacc tggtgtttat tctgttattt acttgtactt |
| 9601 | gacattttat cttactaatg atgtttcttt tttagcacat attcagtgga tggttatgtt |
| 9661 | cacaccttta gtacctttct ggataacaat tgcttatatc atttgtattt ccacaaagca |
| 9721 | tttctattgg ttctttagta attacctaaa gagacgtgta gtctttaatg gtgtttcctt |
| 9781 | tagtactttt gaagaagctg cgctgtgcac ctttttgtta aataaagaaa tgtatctaaa |
| 9841 | gttgcgtagt gatgtgctat tacctcttac gcaatataat agatacttag ctctttataa |
| 9901 | taagtacaag tattttagtg gagcaatgga tacaactagc tacagagaag ctgcttgttg |
| 9961 | tcatctcgca aaggctctca atgacttcag taactcaggt tctgatgttc tttaccaacc |
| 10021 | accacaaacc tctatcacct cagctgtttt gcagagtggt tttagaaaaa tggcattccc |
| 10081 | atctggtaaa gttgagggtt gtatggtaca agtaacttgt ggtacaacta cacttaacgg |
| 10141 | tctttggctt gatgacgtag tttactgtcc aagacatgtg atctgcacct ctgaagacat |
| 10201 | gcttaaccct aattatgaag atttactcat tcgtaagtct aatcataatt tcttggtaca |
| 10261 | ggctggtaat gttcaactca gggttattgg acattctatg caaaattgtg tacttaagct |
| 10321 | taaggttgat acagccaatc ctaagacacc taagtataag tttgttcgca ttcaaccagg |
| 10381 | acagactttt tcagtgttag cttgttacaa tggttcacca tctggtgttt accaatgtgc |
| 10441 | tatgaggccc aatttcacta ttaagggttc attccttaat ggttcatgtg gtagtgttgg |
| 10501 | ttttaacata gattatgact gtgtctcttt ttgttacatg caccatatgg aattaccaac |
| 10561 | tggagttcat gctggcacag acttagaagg taacttttat ggaccttttg ttgacaggca |
| 10621 | aacagcacaa gcagctggta cggacacaac tattacagtt aatgttttag cttggttgta |
| 10681 | cgctgctgtt ataaatggag acaggtggtt tctcaatcga tttaccacaa ctcttaatga |
| 10741 | ctttaacctt gtggctatga agtacaatta tgaacctcta acacaagacc atgttgacat |
| 10801 | actaggacct ctttctgctc aaactggaat tgccgtttta gatatgtgtg cttcattaaa |
| 10861 | agaattactg caaaatggta tgaatggacg taccatattg ggtagtgctt tattagaaga |
| 10921 | tgaatttaca ccttttgatg ttgttagaca atgctcaggt gttactttcc aaagtgcagt |
| 10981 | gaaaagaaca atcaagggta cacaccactg gttgttactc acaattttga cttcactttt |
| 11041 | agttttagtc cagagtactc aatggtcttt gttctttttt ttgtatgaaa atgccttttt |
| 11101 | accttttgct atgggtatta ttgctatgtc tgcttttgca atgatgtttg tcaaacataa |
| 11161 | gcatgcattt ctctgtttgt ttttgttacc ttctcttgcc actgtagctt attttaatat |
| 11221 | ggtctatatg cctgctagtt gggtgatgcg tattatgaca tggttggata tggttgatac |
| 11281 | tagtttgtct ggttttaagc taaaagactg tgttatgtat gcatcagctg tagtgttact |
| 11341 | aatccttatg acagcaagaa ctgtgtatga tgatggtgct aggagagtgt ggacacttat |
| 11401 | gaatgtcttg acactcgttt ataaagttta ttatggtaat gctttagatc aagccatttc |
| 11461 | catgtgggct cttataatct ctgttacttc taactactca ggtgtagtta caactgtcat |
| 11521 | gtttttggcc agaggtattg tttttatgtg tgttgagtat tgccctattt tcttcataac |
| 11581 | tggtaataca cttcagtgta taatgctagt ttattgtttc ttaggctatt tttgtacttg |
| 11641 | ttactttggc ctcttttgtt tactcaaccg ctactttaga ctgactcttg gtgtttatga |
| 11701 | ttacttagtt tctacacagg agtttagata tatgaattca cagggactac tcccacccaa |
| 11761 | gaatagcata gatgccttca aactcaacat taaattgttg ggtgttggtg gcaaaccttg |
| 11821 | tatcaaagta gccactgtac agtctaaaat gtcagatgta aagtgcacat cagtagtctt |
| 11881 | actctcagtt ttgcaacaac tcagagtaga atcatcatct aaattgtggg ctcaatgtgt |
| 11941 | ccagttacac aatgacattc tcttagctaa agatactact gaagcctttg aaaaaatggt |
| 12001 | ttcactactt tctgttttgc tttccatgca gggtgctgta gacataaaca agctttgtga |
| 12061 | agaaatgctg gacaacaggg caaccttaca agctatagcc tcagagttta gttcccttcc |
| 12121 | atcatatgca gcttttgcta ctgctcaaga agcttatgag caggctgttg ctaatggtga |
| 12181 | ttctgaagtt gttcttaaaa agttgaagaa gtctttgaat gtggctaaat ctgaatttga |
| 12241 | ccgtgatgca gccatgcaac gtaagttgga aaagatggct gatcaagcta tgacccaaat |
| 12301 | gtataaacag gctagatctg aggacaagag ggcaaaagtt actagtgcta tgcagacaat |
| 12361 | gcttttcact atgcttagaa agttggataa tgatgcactc aacaacatta tcaacaatgc |
| 12421 | aagagatggt tgtgttccct tgaacataat acctcttaca acagcagcca aactaatggt |
| 12481 | tgtcatacca gactataaca catataaaaa tacgtgtgat ggtacaacat ttacttatgc |
| 12541 | atcagcattg tgggaaatcc aacaggttgt agatgcagat agtaaaattg ttcaacttag |
| 12601 | tgaaattagt atggacaatt cacctaattt agcatggcct cttattgtaa cagctttaag |
| 12661 | ggccaattct gctgtcaaat tacagaataa tgagcttagt cctgttgcac tacgacagat |
| 12721 | gtcttgtgct gccggtacta cacaaactgc ttgcactgat gacaatgcgt tagcttacta |
| 12781 | caacacaaca aagggaggta ggtttgtact tgcactgtta tccgatttac aggatttgaa |
| 12841 | atgggctaga ttccctaaga gtgatggaac tggtactatc tatacagaac tggaaccacc |
| 12901 | ttgtaggttt gttacagaca cacctaaagg tcctaaagtg aagtatttat actttattaa |
| 12961 | aggattaaac aacctaaata gaggtatggt acttggtagt ttagctgcca cagtacgtct |
| 13021 | acaagctggt aatgcaacag aagtgcctgc caattcaact gtattatctt tctgtgcttt |
| 13081 | tgctgtagat gctgctaaag cttacaaaga ttatctagct agtgggggac aaccaatcac |
| 13141 | taattgtgtt aagatgttgt gtacacacac tggtactggt caggcaataa cagttacacc |
| 13201 | ggaagccaat atggatcaag aatcctttgg tggtgcatcg tgttgtctgt actgccgttg |
| 13261 | ccacatagat catccaaatc ctaaaggatt ttgtgactta aaaggtaagt atgtacaaat |
| 13321 | acctacaact tgtgctaatg accctgtggg ttttacactt aaaaacacag tctgtaccgt |
| 13381 | ctgcggtatg tggaaaggtt atggctgtag ttgtgatcaa ctccgcgaac ccatgcttca |
| 13441 | gtcagctgat gcacaatcgt ttttaaacgg gtttgcggtg taagtgcagc ccgtcttaca |
| 13501 | ccgtgcggca caggcactag tactgatgtc gtatacaggg cttttgacat ctacaatgat |
| 13561 | aaagtagctg gttttgctaa attcctaaaa actaattgtt gtcgcttcca agaaaaggac |
| 13621 | gaagatgaca atttaattga ttcttacttt gtagttaaga gacacacttt ctctaactac |
| 13681 | caacatgaag aaacaattta taatttactt aaggattgtc cagctgttgc taaacatgac |
| 13741 | ttctttaagt ttagaataga cggtgacatg gtaccacata tatcacgtca acgtcttact |
| 13801 | aaatacacaa tggcagacct cgtctatgct ttaaggcatt ttgatgaagg taattgtgac |
| 13861 | acattaaaag aaatacttgt cacatacaat tgttgtgatg atgattattt caataaaaag |
| 13921 | gactggtatg attttgtaga aaacccagat atattacgcg tatacgccaa cttaggtgaa |
| 13981 | cgtgtacgcc aagctttgtt aaaaacagta caattctgtg atgccatgcg aaatgctggt |
| 14041 | attgttggtg tactgacatt agataatcaa gatctcaatg gtaactggta tgatttcggt |
| 14101 | gatttcatac aaaccacgcc aggtagtgga gttcctgttg tagattctta ttattcattg |
| 14161 | ttaatgccta tattaacctt gaccagggct ttaactgcag agtcacatgt tgacactgac |
| 14221 | ttaacaaagc cttacattaa gtgggatttg ttaaaatatg acttcacgga agagaggtta |
| 14281 | aaactctttg accgttattt taaatattgg gatcagacat accacccaaa ttgtgttaac |
| 14341 | tgtttggatg acagatgcat tctgcattgt gcaaacttta atgttttatt ctctacagtg |
| 14401 | ttcccaccta caagttttgg accactagtg agaaaaatat ttgttgatgg tgttccattt |
| 14461 | gtagtttcaa ctggatacca cttcagagag ctaggtgttg tacataatca ggatgtaaac |
| 14521 | ttacatagct ctagacttag ttttaaggaa ttacttgtgt atgctgctga ccctgctatg |
| 14581 | cacgctgctt ctggtaatct attactagat aaacgcacta cgtgcttttc agtagctgca |
| 14641 | cttactaaca atgttgcttt tcaaactgtc aaacccggta attttaacaa agacttctat |
| 14701 | gactttgctg tgtctaaggg tttctttaag gaaggaagtt ctgttgaatt aaaacacttc |
| 14761 | ttctttgctc aggatggtaa tgctgctatc agcgattatg actactatcg ttataatcta |
| 14821 | ccaacaatgt gtgatatcag acaactacta tttgtagttg aagttgttga taagtacttt |
| 14881 | gattgttacg atggtggctg tattaatgct aaccaagtca tcgtcaacaa cctagacaaa |
| 14941 | tcagctggtt ttccatttaa taaatggggt aaggctagac tttattatga ttcaatgagt |
| 15001 | tatgaggatc aagatgcact tttcgcatat acaaaacgta atgtcatccc tactataact |
| 15061 | caaatgaatc ttaagtatgc cattagtgca aagaatagag ctcgcaccgt agctggtgtc |
| 15121 | tctatctgta gtactatgac caatagacag tttcatcaaa aattattgaa atcaatagcc |
| 15181 | gccactagag gagctactgt agtaattgga acaagcaaat tctatggtgg ttggcacaac |
| 15241 | atgttaaaaa ctgtttatag tgatgtagaa aaccctcacc ttatgggttg ggattatcct |
| 15301 | aaatgtgata gagccatgcc taacatgctt agaattatgg cctcacttgt tcttgctcgc |
| 15361 | aaacatacaa cgtgttgtag cttgtcacac cgtttctata gattagctaa tgagtgtgct |
| 15421 | caagtattga gtgaaatggt catgtgtggc ggttcactat atgttaaacc aggtggaacc |
| 15481 | tcatcaggag atgccacaac tgcttatgct aatagtgttt ttaacatttg tcaagctgtc |
| 15541 | acggccaatg ttaatgcact tttatctact gatggtaaca aaattgccga taagtatgtc |
| 15601 | cgcaatttac aacacagact ttatgagtgt ctctatagaa atagagatgt tgacacagac |
| 15661 | tttgtgaatg agttttacgc atatttgcgt aaacatttct caatgatgat actctctgac |
| 15721 | gatgctgttg tgtgtttcaa tagcacttat gcatctcaag gtctagtggc tagcataaag |
| 15781 | aactttaagt cagttcttta ttatcaaaac aatgttttta tgtctgaagc aaaatgttgg |
| 15841 | actgagactg accttactaa aggacctcat gaattttgct ctcaacatac aatgctagtt |
| 15901 | aaacagggtg atgattatgt gtaccttcct tacccagatc catcaagaat cctaggggcc |
| 15961 | ggctgttttg tagatgatat cgtaaaaaca gatggtacac ttatgattga acggttcgtg |
| 16021 | tctttagcta tagatgctta cccacttact aaacatccta atcaggagta tgctgatgtc |
| 16081 | tttcatttgt acttacaata cataagaaag ctacatgatg agttaacagg acacatgtta |
| 16141 | gacatgtatt ctgttatgct tactaatgat aacacttcaa ggtattggga acctgagttt |
| 16201 | tatgaggcta tgtacacacc gcatacagtc ttacaggctg ttggggcttg tgttctttgc |
| 16261 | aattcacaga cttcattaag atgtggtgct tgcatacgta gaccattctt atgttgtaaa |
| 16321 | tgctgttacg accatgtcat atcaacatca cataaattag tcttgtctgt taatccgtat |
| 16381 | gtttgcaatg ctccaggttg tgatgtcaca gatgtgactc aactttactt aggaggtatg |
| 16441 | agctattatt gtaaatcaca taaaccaccc attagttttc cattgtgtgc taatggacaa |
| 16501 | gtttttggtt tatataaaaa tacatgtgtt ggtagcgata atgttactga ctttaatgca |
| 16561 | attgcaacat gtgactggac aaatgctggt gattacattt tagctaacac ctgtactgaa |
| 16621 | agactcaagc tttttgcagc agaaacgctc aaagctactg aggagacatt taaactgtct |
| 16681 | tatggtattg ctactgtacg tgaagtgctg tctgacagag aattacatct ttcatgggaa |
| 16741 | gttggtaaac ctagaccacc acttaaccga aattatgtct ttactggtta tcgtgtaact |
| 16801 | aaaaacagta aagtacaaat aggagagtac acctttgaaa aaggtgacta tggtgatgct |
| 16861 | gttgtttacc gaggtacaac aacttacaaa ttaaatgttg gtgattattt tgtgctgaca |
| 16921 | tcacatacag taatgccatt aagtgcacct acactagtgc cacaagagca ctatgttaga |
| 16981 | attactggct tatacccaac actcaatatc tcagatgagt tttctagcaa tgttgcaaat |
| 17041 | tatcaaaagg ttggtatgca aaagtattct acactccagg gaccacctgg tactggtaag |
| 17101 | agtcattttg ctattggcct agctctctac tacccttctg ctcgcatagt gtatacagct |
| 17161 | tgctctcatg ccgctgttga tgcactatgt gagaaggcat taaaatattt gcctatagat |
| 17221 | aaatgtagta gaattatacc tgcacgtgct cgtgtagagt gttttgataa attcaaagtg |
| 17281 | aattcaacat tagaacagta tgtcttttgt actgtaaatg cattgcctga gacgacagca |
| 17341 | gatatagttg tctttgatga aatttcaatg gccacaaatt atgatttgag tgttgtcaat |
| 17401 | gccagattac gtgctaagca ctatgtgtac attggcgacc ctgctcaatt acctgcacca |
| 17461 | cgcacattgc taactaaggg cacactagaa ccagaatatt tcaattcagt gtgtagactt |
| 17521 | atgaaaacta taggtccaga catgttcctc ggaacttgtc ggcgttgtcc tgctgaaatt |
| 17581 | gttgacactg tgagtgcttt ggtttatgat aataagctta aagcacataa agacaaatca |
| 17641 | gctcaatgct ttaaaatgtt ttataagggt gttatcacgc atgatgtttc atctgcaatt |
| 17701 | aacaggccac aaataggcgt ggtaagagaa ttccttacac gtaaccctgc ttggagaaaa |
| 17761 | gctgtcttta tttcacctta taattcacag aatgctgtag cctcaaagat tttgggacta |
| 17821 | ccaactcaaa ctgttgattc atcacagggc tcagaatatg actatgtcat attcactcaa |
| 17881 | accactgaaa cagctcactc ttgtaatgta aacagattta atgttgctat taccagagca |
| 17941 | aaagtaggca tactttgcat aatgtctgat agagaccttt atgacaagtt gcaatttaca |
| 18001 | agtcttgaaa ttccacgtag gaatgtggca actttacaag ctgaaaatgt aacaggactt |
| 18061 | tttaaagatt gtagtaaggt aatcactggg ttacatccta cacaggcacc tacacacctc |
| 18121 | agtgttgaca ctaaattcaa aactgaaggt ttatgtgttg acatacctgg catacctaag |
| 18181 | gacatgacct atagaagact catctctatg atgggtttta aaatgaatta tcaagttaat |
| 18241 | ggttacccta acatgtttat cacccgcgaa gaagctataa gacatgtacg tgcatggatt |
| 18301 | ggcttcgatg tcgaggggtg tcatgctact agagaagctg ttggtaccaa tttaccttta |
| 18361 | cagctaggtt tttctacagg tgttaaccta gttgctgtac ctacaggtta tgttgataca |
| 18421 | cctaataata cagatttttc cagagttagt gctaaaccac cgcctggaga tcaatttaaa |
| 18481 | cacctcatac cacttatgta caaaggactt ccttggaatg tagtgcgtat aaagattgta |
| 18541 | caaatgttaa gtgacacact taaaaatctc tctgacagag tcgtatttgt cttatgggca |
| 18601 | catggctttg agttgacatc tatgaagtat tttgtgaaaa taggacctga gcgcacctgt |
| 18661 | tgtctatgtg atagacgtgc cacatgcttt tccactgctt cagacactta tgcctgttgg |
| 18721 | catcattcta ttggatttga ttacgtctat aatccgttta tgattgatgt tcaacaatgg |
| 18781 | ggttttacag gtaacctaca aagcaaccat gatctgtatt gtcaagtcca tggtaatgca |
| 18841 | catgtagcta gttgtgatgc aatcatgact aggtgtctag ctgtccacga gtgctttgtt |
| 18901 | aagcgtgttg actggactat tgaatatcct ataattggtg atgaactgaa gattaatgcg |
| 18961 | gcttgtagaa aggttcaaca catggttgtt aaagctgcat tattagcaga caaattccca |
| 19021 | gttcttcacg acattggtaa ccctaaagct attaagtgtg tacctcaagc tgatgtagaa |
| 19081 | tggaagttct atgatgcaca gccttgtagt gacaaagctt ataaaataga agaattattc |
| 19141 | tattcttatg ccacacattc tgacaaattc acagatggtg tatgcctatt ttggaattgc |
| 19201 | aatgtcgata gatatcctgc taattccatt gtttgtagat ttgacactag agtgctatct |
| 19261 | aaccttaact tgcctggttg tgatggtggc agtttgtatg taaataaaca tgcattccac |
| 19321 | acaccagctt ttgataaaag tgcttttgtt aatttaaaac aattaccatt tttctattac |
| 19381 | tctgacagtc catgtgagtc tcatggaaaa caagtagtgt cagatataga ttatgtacca |
| 19441 | ctaaagtctg ctacgtgtat aacacgttgc aatttaggtg gtgctgtctg tagacatcat |
| 19501 | gctaatgagt acagattgta tctcgatgct tataacatga tgatctcagc tggctttagc |
| 19561 | ttgtgggttt acaaacaatt tgatacttat aacctctgga acacttttac aagacttcag |
| 19621 | agtttagaaa atgtggcttt taatgttgta aataagggac actttgatgg acaacagggt |
| 19681 | gaagtaccag tttctatcat taataacact gtttacacaa aagttgatgg tgttgatgta |
| 19741 | gaattgtttg aaaataaaac aacattacct gttaatgtag catttgagct ttgggctaag |
| 19801 | cgcaacatta aaccagtacc agaggtgaaa atactcaata atttgggtgt ggacattgct |
| 19861 | gctaatactg tgatctggga ctacaaaaga gatgctccag cacatatatc tactattggt |
| 19921 | gtttgttcta tgactgacat agccaagaaa ccaactgaaa cgatttgtgc accactcact |
| 19981 | gtcttttttg atggtagagt tgatggtcaa gtagacttat ttagaaatgc ccgtaatggt |
| 20041 | gttcttatta cagaaggtag tgttaaaggt ttacaaccat ctgtaggtcc caaacaagct |
| 20101 | agtcttaatg gagtcacatt aattggagaa gccgtaaaaa cacagttcaa ttattataag |
| 20161 | aaagttgatg gtgttgtcca acaattacct gaaacttact ttactcagag tagaaattta |
| 20221 | caagaattta aacccaggag tcaaatggaa attgatttct tagaattagc tatggatgaa |
| 20281 | ttcattgaac ggtataaatt agaaggctat gccttcgaac atatcgttta tggagatttt |
| 20341 | agtcatagtc agttaggtgg tttacatcta ctgattggac tagctaaacg ttttaaggaa |
| 20401 | tcaccttttg aattagaaga ttttattcct atggacagta cagttaaaaa ctatttcata |
| 20461 | acagatgcgc aaacaggttc atctaagtgt gtgtgttctg ttattgattt attacttgat |
| 20521 | gattttgttg aaataataaa atcccaagat ttatctgtag tttctaaggt tgtcaaagtg |
| 20581 | actattgact atacagaaat ttcatttatg ctttggtgta aagatggcca tgtagaaaca |
| 20641 | ttttacccaa aattacaatc tagtcaagcg tggcaaccgg gtgttgctat gcctaatctt |
| 20701 | tacaaaatgc aaagaatgct attagaaaag tgtgaccttc aaaattatgg tgatagtgca |
| 20761 | acattaccta aaggcataat gatgaatgtc gcaaaatata ctcaactgtg tcaatattta |
| 20821 | aacacattaa cattagctgt accctataat atgagagtta tacattttgg tgctggttct |
| 20881 | gataaaggag ttgcaccagg tacagctgtt ttaagacagt ggttgcctac gggtacgctg |
| 20941 | cttgtcgatt cagatcttaa tgactttgtc tctgatgcag attcaacttt gattggtgat |
| 21001 | tgtgcaactg tacatacagc taataaatgg gatctcatta ttagtgatat gtacgaccct |
| 21061 | aagactaaaa atgttacaaa agaaaatgac tctaaagagg gttttttcac ttacatttgt |
| 21121 | gggtttatac aacaaaagct agctcttgga ggttccgtgg ctataaagat aacagaacat |
| 21181 | tcttggaatg ctgatcttta taagctcatg ggacacttcg catggtggac agcctttgtt |
| 21241 | actaatgtga atgcgtcatc atctgaagca tttttaattg gatgtaatta tcttggcaaa |
| 21301 | ccacgcgaac aaatagatgg ttatgtcatg catgcaaatt acatattttg gaggaataca |
| 21361 | aatccaattc agttgtcttc ctattcttta tttgacatga gtaaatttcc ccttaaatta |
| 21421 | aggggtactg ctgttatgtc tttaaaagaa ggtcaaatca atgatatgat tttatctctt |
| 21481 | cttagtaaag gtagacttat aattagagaa aacaacagag ttgttatttc tagtgatgtt |
| 21541 | cttgttaaca actaaacgaa caatgtttgt ttttcttgtt ttattgccac tagtctctag |
| 21601 | tcagtgtgtt aatcttacaa ccagaactca attaccccct gcatacacta attctttcac |
| 21661 | acgtggtgtt tattaccctg acaaagtttt cagatcctca gttttacatt caactcagga |
| 21721 | cttgttctta cctttctttt ccaatgttac ttggttccat gctatacatg tctctgggac |
| 21781 | caatggtact aagaggtttg ataaccctgt cctaccattt aatgatggtg tttattttgc |
| 21841 | ttccactgag aagtctaaca taataagagg ctggattttt ggtactactt tagattcgaa |
| 21901 | gacccagtcc ctacttattg ttaataacgc tactaatgtt gttattaaag tctgtgaatt |
| 21961 | tcaattttgt aatgatccat ttttgggtgt ttattaccac aaaaacaaca aaagttggat |
| 22021 | ggaaagtgag ttcagagttt attctagtgc gaataattgc acttttgaat atgtctctca |
| 22081 | gccttttctt atggaccttg aaggaaaaca gggtaatttc aaaaatctta gggaatttgt |
| 22141 | gtttaagaat attgatggtt attttaaaat atattctaag cacacgccta ttaatttagt |
| 22201 | gcgtgatctc cctcagggtt tttcggcttt agaaccattg gtagatttgc caataggtat |
| 22261 | taacatcact aggtttcaaa ctttacttgc tttacataga agttatttga ctcctggtga |
| 22321 | ttcttcttca ggttggacag ctggtgctgc agcttattat gtgggttatc ttcaacctag |
| 22381 | gacttttcta ttaaaatata atgaaaatgg aaccattaca gatgctgtag actgtgcact |
| 22441 | tgaccctctc tcagaaacaa agtgtacgtt gaaatccttc actgtagaaa aaggaatcta |
| 22501 | tcaaacttct aactttagag tccaaccaac agaatctatt gttagatttc ctaatattac |
| 22561 | aaacttgtgc ccttttggtg aagtttttaa cgccaccaga tttgcatctg tttatgcttg |
| 22621 | gaacaggaag agaatcagca actgtgttgc tgattattct gtcctatata attccgcatc |
| 22681 | attttccact tttaagtgtt atggagtgtc tcctactaaa ttaaatgatc tctgctttac |
| 22741 | taatgtctat gcagattcat ttgtaattag aggtgatgaa gtcagacaaa tcgctccagg |
| 22801 | gcaaactgga aagattgctg attataatta taaattacca gatgatttta caggctgcgt |
| 22861 | tatagcttgg aattctaaca atcttgattc taaggttggt ggtaattata attacctgta |
| 22921 | tagattgttt aggaagtcta atctcaaacc ttttgagaga gatatttcaa ctgaaatcta |
| 22981 | tcaggccggt agcacacctt gtaatggtgt tgaaggtttt aattgttact ttcctttaca |
| 23041 | atcatatggt ttccaaccca ctaatggtgt tggttaccaa ccatacagag tagtagtact |
| 23101 | ttcttttgaa cttctacatg caccagcaac tgtttgtgga cctaaaaagt ctactaattt |
| 23161 | ggttaaaaac aaatgtgtca atttcaactt caatggttta acaggcacag gtgttcttac |
| 23221 | tgagtctaac aaaaagtttc tgcctttcca acaatttggc agagacattg ctgacactac |
| 23281 | tgatgctgtc cgtgatccac agacacttga gattcttgac attacaccat gttcttttgg |
| 23341 | tggtgtcagt gttataacac caggaacaaa tacttctaac caggttgctg ttctttatca |
| 23401 | ggatgttaac tgcacagaag tccctgttgc tattcatgca gatcaactta ctcctacttg |
| 23461 | gcgtgtttat tctacaggtt ctaatgtttt tcaaacacgt gcaggctgtt taataggggc |
| 23521 | tgaacatgtc aacaactcat atgagtgtga catacccatt ggtgcaggta tatgcgctag |
| 23581 | ttatcagact cagactaatt ctcctcggcg ggcacgtagt gtagctagtc aatccatcat |
| 23641 | tgcctacact atgtcacttg gtgcagaaaa ttcagttgct tactctaata actctattgc |
| 23701 | catacccaca aattttacta ttagtgttac cacagaaatt ctaccagtgt ctatgaccaa |
| 23761 | gacatcagta gattgtacaa tgtacatttg tggtgattca actgaatgca gcaatctttt |
| 23821 | gttgcaatat ggcagttttt gtacacaatt aaaccgtgct ttaactggaa tagctgttga |
| 23881 | acaagacaaa aacacccaag aagtttttgc acaagtcaaa caaatttaca aaacaccacc |
| 23941 | aattaaagat tttggtggtt ttaatttttc acaaatatta ccagatccat caaaaccaag |
| 24001 | caagaggtca tttattgaag atctactttt caacaaagtg acacttgcag atgctggctt |
| 24061 | catcaaacaa tatggtgatt gccttggtga tattgctgct agagacctca tttgtgcaca |
| 24121 | aaagtttaac ggccttactg ttttgccacc tttgctcaca gatgaaatga ttgctcaata |
| 24181 | cacttctgca ctgttagcgg gtacaatcac ttctggttgg acctttggtg caggtgctgc |
| 24241 | attacaaata ccatttgcta tgcaaatggc ttataggttt aatggtattg gagttacaca |
| 24301 | gaatgttctc tatgagaacc aaaaattgat tgccaaccaa tttaatagtg ctattggcaa |
| 24361 | aattcaagac tcactttctt ccacagcaag tgcacttgga aaacttcaag atgtggtcaa |
| 24421 | ccaaaatgca caagctttaa acacgcttgt taaacaactt agctccaatt ttggtgcaat |
| 24481 | ttcaagtgtt ttaaatgata tcctttcacg tcttgacaaa gttgaggctg aagtgcaaat |
| 24541 | tgataggttg atcacaggca gacttcaaag tttgcagaca tatgtgactc aacaattaat |
| 24601 | tagagctgca gaaatcagag cttctgctaa tcttgctgct actaaaatgt cagagtgtgt |
| 24661 | acttggacaa tcaaaaagag ttgatttttg tggaaagggc tatcatctta tgtccttccc |
| 24721 | tcagtcagca cctcatggtg tagtcttctt gcatgtgact tatgtccctg cacaagaaaa |
| 24781 | gaacttcaca actgctcctg ccatttgtca tgatggaaaa gcacactttc ctcgtgaagg |
| 24841 | tgtctttgtt tcaaatggca cacactggtt tgtaacacaa aggaattttt atgaaccaca |
| 24901 | aatcattact acagacaaca catttgtgtc tggtaactgt gatgttgtaa taggaattgt |
| 24961 | caacaacaca gtttatgatc ctttgcaacc tgaattagac tcattcaagg aggagttaga |
| 25021 | taaatatttt aagaatcata catcaccaga tgttgattta ggtgacatct ctggcattaa |
| 25081 | tgcttcagtt gtaaacattc aaaaagaaat tgaccgcctc aatgaggttg ccaagaattt |
| 25141 | aaatgaatct ctcatcgatc tccaagaact tggaaagtat gagcagtata taaaatggcc |
| 25201 | atggtacatt tggctaggtt ttatagctgg cttgattgcc atagtaatgg tgacaattat |
| 25261 | gctttgctgt atgaccagtt gctgtagttg tctcaagggc tgttgttctt gtggatcctg |
| 25321 | ctgcaaattt gatgaagacg actctgagcc agtgctcaaa ggagtcaaat tacattacac |
| 25381 | ataaacgaac ttatggattt gtttatgaga atcttcacaa ttggaactgt aactttgaag |
| 25441 | caaggtgaaa tcaaggatgc tactccttca gattttgttc gcgctactgc aacgataccg |
| 25501 | atacaagcct cactcccttt cggatggctt attgttggcg ttgcacttct tgctgttttt |
| 25561 | cagagcgctt ccaaaatcat aaccctcaaa aagagatggc aactagcact ctccaagggt |
| 25621 | gttcactttg tttgcaactt gctgttgttg tttgtaacag tttactcaca ccttttgctc |
| 25681 | gttgctgctg gccttgaagc cccttttctc tatctttatg ctttagtcta cttcttgcag |
| 25741 | agtataaact ttgtaagaat aataatgagg ctttggcttt gctggaaatg ccgttccaaa |
| 25801 | aacccattac tttatgatgc caactatttt ctttgctggc atactaattg ttacgactat |
| 25861 | tgtatacctt acaatagtgt aacttcttca attgtcatta cttcaggtga tggcacaaca |
| 25921 | agtcctattt ctgaacatga ctaccagatt ggtggttata ctgaaaaatg ggaatctgga |
| 25981 | gtaaaagact gtgttgtatt acacagttac ttcacttcag actattacca gctgtactca |
| 26041 | actcaattga gtacagacac tggtgttgaa catgttacct tcttcatcta caataaaatt |
| 26101 | gttgatgagc ctgaagaaca tgtccaaatt cacacaatcg acggttcatc cggagttgtt |
| 26161 | aatccagtaa tggaaccaat ttatgatgaa ccgacgacga ctactagcgt gcctttgtaa |
| 26221 | gcacaagctg atgagtacga acttatgtac tcattcgttt cggaagagac aggtacgtta |
| 26281 | atagttaata gcgtacttct ttttcttgct ttcgtggtat tcttgctagt tacactagcc |
| 26341 | atccttactg cgcttcgatt gtgtgcgtac tgctgcaata ttgttaacgt gagtcttgta |
| 26401 | aaaccttctt tttacgttta ctctcgtgtt aaaaatctga attcttctag agttcctgat |
| 26461 | cttctggtct aaacgaacta aatattatat tagtttttct gtttggaact ttaattttag |
| 26521 | ccatggcaga ttccaacggt actattaccg ttgaagagct taaaaagctc cttgaacaat |
| 26581 | ggaacctagt aataggtttc ctattcctta catggatttg tcttctacaa tttgcctatg |
| 26641 | ccaacaggaa taggtttttg tatataatta agttaatttt cctctggctg ttatggccag |
| 26701 | taactttagc ttgttttgtg cttgctgctg tttacagaat aaattggatc accggtggaa |
| 26761 | ttgctatcgc aatggcttgt cttgtaggct tgatgtggct cagctacttc attgcttctt |
| 26821 | tcagactgtt tgcgcgtacg cgttccatgt ggtcattcaa tccagaaact aacattcttc |
| 26881 | tcaacgtgcc actccatggc actattctga ccagaccgct tctagaaagt gaactcgtaa |
| 26941 | tcggagctgt gatccttcgt ggacatcttc gtattgctgg acaccatcta ggacgctgtg |
| 27001 | acatcaagga cctgcctaaa gaaatcactg ttgctacatc acgaacgctt tcttattaca |
| 27061 | aattgggagc ttcgcagcgt gtagcaggtg actcaggttt tgctgcatac agtcgctaca |
| 27121 | ggattggcaa ctataaatta aacacagacc attccagtag cagtgacaat attgctttgc |
| 27181 | ttgtacagta agtgacaaca gatgtttcat ctcgttgact ttcaggttac tatagcagag |
| 27241 | atattactaa ttattatgag gacttttaaa gtttccattt ggaatcttga ttacatcata |
| 27301 | aacctcataa ttaaaaattt atctaagtca ctaactgaga ataaatattc tcaattagat |
| 27361 | gaagagcaac caatggagat tgattaaacg aacatgaaaa ttattctttt cttggcactg |
| 27421 | ataacactcg ctacttgtga gctttatcac taccaagagt gtgttagagg tacaacagta |
| 27481 | cttttaaaag aaccttgctc ttctggaaca tacgagggca attcaccatt tcatcctcta |
| 27541 | gctgataaca aatttgcact gacttgcttt agcactcaat ttgcttttgc ttgtcctgac |
| 27601 | ggcgtaaaac acgtctatca gttacgtgcc agatcagttt cacctaaact gttcatcaga |
| 27661 | caagaggaag ttcaagaact ttactctcca atttttctta ttgttgcggc aatagtgttt |
| 27721 | ataacacttt gcttcacact caaaagaaag acagaatgat tgaactttca ttaattgact |
| 27781 | tctatttgtg ctttttagcc tttctgctat tccttgtttt aattatgctt attatctttt |
| 27841 | ggttctcact tgaactgcaa gatcataatg aaacttgtca cgcctaaacg aacatgaaat |
| 27901 | ttcttgtttt cttaggaatc atcacaactg tagctgcatt tcaccaagaa tgtagtttac |
| 27961 | agtcatgtac tcaacatcaa ccatatgtag ttgatgaccc gtgtcctatt cacttctatt |
| 28021 | ctaaatggta tattagagta ggagctagaa aatcagcacc tttaattgaa ttgtgcgtgg |
| 28081 | atgaggctgg ttctaaatca cccattcagt acatcgatat cggtaattat acagtttcct |
| 28141 | gttcaccttt tacaattaat tgccaggaac ctaaattggg tagtcttgta gtgcgttgtt |
| 28201 | cgttctatga agacttttta gagtatcatg acgttcgtgt tgttttagat ttcatctaaa |
| 28261 | cgaacaaact aaaatgtctg ataatggacc ccaaaatcag cgaaatgcac cccgcattac |
| 28321 | gtttggtgga ccctcagatt caactggcag taaccagaat ggagaacgca gtggggcgcg |
| 28381 | atcaaaacaa cgtcggcccc aaggtttacc caataatact gcgtcttggt tcaccgctct |
| 28441 | cactcaacat ggcaaggaag accttaaatt ccctcgagga caaggcgttc caattaacac |
| 28501 | caatagcagt ccagatgacc aaattggcta ctaccgaaga gctaccagac gaattcgtgg |
| 28561 | tggtgacggt aaaatgaaag atctcagtcc aagatggtat ttctactacc taggaactgg |
| 28621 | gccagaagct ggacttccct atggtgctaa caaagacggc atcatatggg ttgcaactga |
| 28681 | gggagccttg aatacaccaa aagatcacat tggcacccgc aatcctgcta acaatgctgc |
| 28741 | aatcgtgcta caacttcctc aaggaacaac attgccaaaa ggcttctacg cagaagggag |
| 28801 | cagaggcggc agtcaagcct cttctcgttc ctcatcacgt agtcgcaaca gttcaagaaa |
| 28861 | ttcaactcca ggcagcagta ggggaacttc tcctgctaga atggctggca atggcggtga |
| 28921 | tgctgctctt gctttgctgc tgcttgacag attgaaccag cttgagagca aaatgtctgg |
| 28981 | taaaggccaa caacaacaag gccaaactgt cactaagaaa tctgctgctg aggcttctaa |
| 29041 | gaagcctcgg caaaaacgta ctgccactaa agcatacaat gtaacacaag ctttcggcag |
| 29101 | acgtggtcca gaacaaaccc aaggaaattt tggggaccag gaactaatca gacaaggaac |
| 29161 | tgattacaaa cattggccgc aaattgcaca atttgccccc agcgcttcag cgttcttcgg |
| 29221 | aatgtcgcgc attggcatgg aagtcacacc ttcgggaacg tggttgacct acacaggtgc |
| 29281 | catcaaattg gatgacaaag atccaaattt caaagatcaa gtcattttgc tgaataagca |
| 29341 | tattgacgca tacaaaacat tcccaccaac agagcctaaa aaggacaaaa agaagaaggc |
| 29401 | tgatgaaact caagccttac cgcagagaca gaagaaacag caaactgtga ctcttcttcc |
| 29461 | tgctgcagat ttggatgatt tctccaaaca attgcaacaa tccatgagca gtgctgactc |
| 29521 | aactcaggcc taaactcatg cagaccacac aaggcagatg ggctatataa acgttttcgc |
| 29581 | ttttccgttt acgatatata gtctactctt gtgcagaatg aattctcgta actacatagc |
| 29641 | acaagtagat gtagttaact ttaatctcac atagcaatct ttaatcagtg tgtaacatta |
| 29701 | gggaggactt gaaagagcca ccacattttc accgaggcca cgcggagtac gatcgagtgt |
| 29761 | acagtgaaca atgctaggga gagctgccta tatggaagag ccctaatgtg taaaattaat |
| 29821 | tttagtagtg ctatccccat gtgattttaa tagcttctta ggagaatgac aaaaaaaaaa |
| 29881 | aa. |

GenBank Accession No. GenBank: AY274119.3, which is specifically incorporated by reference herein in its entirety, provides the following (DNA) genomic sequence for SARS-CoV (Severe acute respiratory syndrome-related coronavirus isolate Tor2, complete genome):

| (SEQ ID NO: 3) | |
|---|---|
| 1 | atattaggtt tttacctacc caggaaaagc caaccaacct cgatctcttg tagatctgtt |
| 61 | ctctaaacga actttaaaat ctgtgtagct gtcgctcggc tgcatgccta gtgcacctac |
| 121 | gcagtataaa caataataaa ttttactgtc gttgacaaga aacgagtaac tcgtccctct |
| 181 | tctgcagact gcttacggtt tcgtccgtgt tgcagtcgat catcagcata cctaggtttc |
| 241 | gtccgggtgt gaccgaaagg taagatggag agccttgttc ttggtgtcaa cgagaaaaca |
| 301 | cacgtccaac tcagtttgcc tgtccttcag gttagagacg tgctagtgcg tggcttcggg |
| 361 | gactctgtgg aagaggccct atcggaggca cgtgaacacc tcaaaaatgg cacttgtggt |
| 421 | ctagtagagc tggaaaaagg cgtactgccc cagcttgaac agccctatgt gttcattaaa |
| 481 | cgttctgatg ccttaagcac caatcacggc cacaaggtcg ttgagctggt tgcagaaatg |
| 541 | gacggcattc agtacggtcg tagcggtata acactgggag tactcgtgcc acatgtgggc |
| 601 | gaaaccccaa ttgcataccg caatgttctt cttcgtaaga acggtaataa gggagccggt |
| 661 | ggtcatagct atggcatcga tctaaagtct tatgacttag gtgacgagct tggcactgat |
| 721 | cccattgaag attatgaaca aaactggaac actaagcatg gcagtggtgc actccgtgaa |
| 781 | ctcactcgtg agctcaatgg aggtgcagtc actcgctatg tcgacaacaa tttctgtggc |
| 841 | ccagatgggt accctcttga ttgcatcaaa gattttctcg cacgcgcggg caagtcaatg |
| 901 | tgcactcttt ccgaacaact tgattacatc gagtcgaaga gaggtgtcta ctgctgccgt |
| 961 | gaccatgagc atgaaattgc ctggttcact gagcgctctg ataagagcta cgagcaccag |
| 1021 | acacccttcg aaattaagag tgccaagaaa tttgacactt tcaaagggga atgcccaaag |
| 1081 | tttgtgtttc ctcttaactc aaaagtcaaa gtcattcaac cacgtgttga aaagaaaaag |
| 1141 | actgagggtt tcatggggcg tatacgctct gtgtaccctg ttgcatctcc acaggagtgt |
| 1201 | aacaatatgc acttgtctac cttgatgaaa tgtaatcatt gcgatgaagt ttcatggcag |
| 1261 | acgtgcgact ttctgaaagc cacttgtgaa cattgtggca ctgaaaattt agttattgaa |
| 1321 | ggacctacta catgtgggta cctacctact aatgctgtag tgaaaatgcc atgtcctgcc |
| 1381 | tgtcaagacc cagagattgg acctgagcat agtgttgcag attatcacaa ccactcaaac |
| 1441 | attgaaactc gactccgcaa gggaggtagg actagatgtt ttggaggctg tgtgtttgcc |
| 1501 | tatgttggct gctataataa gcgtgcctac tgggttcctc gtgctagtgc tgatattggc |
| 1561 | tcaggccata ctggcattac tggtgacaat gtggagacct tgaatgagga tctccttgag |
| 1621 | atactgagtc gtgaacgtgt taacattaac attgttggcg attttcattt gaatgaagag |
| 1681 | gttgccatca ttttggcatc tttctctgct tctacaagtg cctttattga cactataaag |
| 1741 | agtcttgatt acaagtcttt caaaaccatt gttgagtcct gcggtaacta taaagttacc |
| 1801 | aagggaaagc ccgtaaaagg tgcttggaac attggacaac agagatcagt tttaacacca |
| 1861 | ctgtgtggtt ttccctcaca ggctgctggt gttatcagat caatttttgc gcgcacactt |
| 1921 | gatgcagcaa accactcaat tcctgatttg caaagagcag ctgtcaccat acttgatggt |
| 1981 | atttctgaac agtcattacg tcttgtcgac gccatggttt atacttcaga cctgctcacc |
| 2041 | aacagtgtca ttattatggc atatgtaact ggtggtcttg tacaacagac ttctcagtgg |
| 2101 | ttgtctaatc ttttgggcac tactgttgaa aaactcaggc ctatctttga atggattgag |
| 2161 | gcgaaactta gtgcaggagt tgaatttctc aaggatgctt gggagattct caaatttctc |
| 2221 | attacaggtg tttttgacat cgtcaagggt caaatacagg ttgcttcaga taacatcaag |
| 2281 | gattgtgtaa aatgcttcat tgatgttgtt aacaaggcac tcgaaatgtg cattgatcaa |
| 2341 | gtcactatcg ctggcgcaaa gttgcgatca ctcaacttag gtgaagtctt catcgctcaa |
| 2401 | agcaagggac tttaccgtca gtgtatacgt ggcaaggagc agctgcaact actcatgcct |
| 2461 | cttaaggcac caaaagaagt aacctttctt gaaggtgatt cacatgacac agtacttacc |
| 2521 | tctgaggagg ttgttctcaa gaacggtgaa ctcgaagcac tcgagacgcc cgttgatagc |
| 2581 | ttcacaaatg gagctatcgt tggcacacca gtctgtgtaa atggcctcat gctcttagag |
| 2641 | attaaggaca aagaacaata ctgcgcattg tctcctggtt tactggctac aaacaatgtc |
| 2701 | tttcgcttaa aagggggtgc accaattaaa ggtgtaacct ttggagaaga tactgtttgg |
| 2761 | gaagttcaag gttacaagaa tgtgagaatc acatttgagc ttgatgaacg tgttgacaaa |
| 2821 | gtgcttaatg aaaagtgctc tgtctacact gttgaatccg gtaccgaagt tactgagttt |
| 2881 | gcatgtgttg tagcagaggc tgttgtgaag actttacaac cagtttctga tctccttacc |
| 2941 | aacatgggta ttgatcttga tgagtggagt gtagctacat tctacttatt tgatgatgct |
| 3001 | ggtgaagaaa acttttcatc acgtatgtat tgttcctttt accctccaga tgaggaagaa |
| 3061 | gaggacgatg cagagtgtga ggaagaagaa attgatgaaa cctgtgaaca tgagtacggt |
| 3121 | acagaggatg attatcaagg tctccctctg gaatttggtg cctcagctga aacagttcga |
| 3181 | gttgaggaag aagaagagga agactggctg gatgatacta ctgagcaatc agagattgag |
| 3241 | ccagaaccag aacctacacc tgaagaacca gttaatcagt ttactggtta tttaaaactt |
| 3301 | actgacaatg ttgccattaa atgtgttgac atcgttaagg aggcacaaag tgctaatcct |
| 3361 | atggtgattg taaatgctgc taacatacac ctgaaacatg gtggtggtgt agcaggtgca |
| 3421 | ctcaacaagg caaccaatgg tgccatgcaa aaggagagtg atgattacat taagctaaat |
| 3481 | ggccctctta cagtaggagg gtcttgtttg ctttctggac ataatcttgc taagaagtgt |
| 3541 | ctgcatgttg ttggacctaa cctaaatgca ggtgaggaca tccagcttct taaggcagca |
| 3601 | tatgaaaatt tcaattcaca ggacatctta cttgcaccat tgttgtcagc aggcatattt |
| 3661 | ggtgctaaac cacttcagtc tttacaagtg tgcgtgcaga cggttcgtac acaggtttat |
| 3721 | attgcagtca atgacaaagc tctttatgag caggttgtca tggattatct tgataacctg |
| 3781 | aagcctagag tggaagcacc taaacaagag gagccaccaa acacagaaga ttccaaaact |
| 3841 | gaggagaaat ctgtcgtaca gaagcctgtc gatgtgaagc caaaaattaa ggcctgcatt |
| 3901 | gatgaggtta ccacaacact ggaagaaact aagtttctta ccaataagtt actcttgttt |
| 3961 | gctgatatca atggtaagct ttaccatgat tctcagaaca tgcttagagg tgaagatatg |
| 4021 | tctttccttg agaaggatgc accttacatg gtaggtgatg ttatcactag tggtgatatc |
| 4081 | acttgtgttg taataccctc caaaaaggct ggtggcacta ctgagatgct ctcaagagct |
| 4141 | ttgaagaaag tgccagttga tgagtatata accacgtacc ctggacaagg atgtgctggt |
| 4201 | tatacacttg aggaagctaa gactgctctt aagaaatgca aatctgcatt ttatgtacta |
| 4261 | ccttcagaag cacctaatgc taaggaagag attctaggaa ctgtatcctg gaatttgaga |
| 4321 | gaaatgcttg ctcatgctga agagacaaga aaattaatgc ctatatgcat ggatgttaga |
| 4381 | gccataatgg caaccatcca acgtaagtat aaaggaatta aaattcaaga gggcatcgtt |
| 4441 | gactatggtg tccgattctt cttttatact agtaaagagc ctgtagcttc tattattacg |
| 4501 | aagctgaact ctctaaatga gccgcttgtc acaatgccaa ttggttatgt gacacatggt |
| 4561 | tttaatcttg aagaggctgc gcgctgtatg cgttctctta aagctcctgc cgtagtgtca |
| 4621 | gtatcatcac cagatgctgt tactacatat aatggatacc tcacttcgtc atcaaagaca |
| 4681 | tctgaggagc actttgtaga aacagtttct ttggctggct cttacagaga ttggtcctat |
| 4741 | tcaggacagc gtacagagtt aggtgttgaa tttcttaagc gtggtgacaa aattgtgtac |
| 4801 | cacactctgg agagccccgt cgagtttcat cttgacggtg aggttctttc acttgacaaa |
| 4861 | ctaaagagtc tcttatccct gcgggaggtt aagactataa aagtgttcac aactgtggac |
| 4921 | aacactaatc tccacacaca gcttgtggat atgtctatga catatggaca gcagtttggt |
| 4981 | ccaacatact tggatggtgc tgatgttaca aaaattaaac ctcatgtaaa tcatgagggt |
| 5041 | aagactttct ttgtactacc tagtgatgac acactacgta gtgaagcttt cgagtactac |
| 5101 | catactcttg atgagagttt tcttggtagg tacatgtctg ctttaaacca cacaaagaaa |
| 5161 | tggaaatttc ctcaagttgg tggtttaact tcaattaaat gggctgataa caattgttat |
| 5221 | ttgtctagtg ttttattagc acttcaacag cttgaagtca aattcaatgc accagcactt |
| 5281 | caagaggctt attatagagc ccgtgctggt gatgctgcta acttttgtgc actcatactc |
| 5341 | gcttacagta ataaaactgt tggcgagctt ggtgatgtca gagaaactat gacccatctt |
| 5401 | ctacagcatg ctaatttgga atctgcaaag cgagttctta atgtggtgtg taaacattgt |
| 5461 | ggtcagaaaa ctactacctt aacgggtgta gaagctgtga tgtatatggg tactctatct |
| 5521 | tatgataatc ttaagacagg tgtttccatt ccatgtgtgt gtggtcgtga tgctacacaa |
| 5581 | tatctagtac aacaagagtc ttcttttgtt atgatgtctg caccacctgc tgagtataaa |
| 5641 | ttacagcaag gtacattctt atgtgcgaat gagtacactg gtaactatca gtgtggtcat |
| 5701 | tacactcata taactgctaa ggagaccctc tatcgtattg acggagctca ccttacaaag |
| 5761 | atgtcagagt acaaaggacc agtgactgat gttttctaca aggaaacatc ttacactaca |
| 5821 | accatcaagc ctgtgtcgta taaactcgat ggagttactt acacagagat tgaaccaaaa |
| 5881 | ttggatgggt attataaaaa ggataatgct tactatacag agcagcctat agaccttgta |
| 5941 | ccaactcaac cattaccaaa tgcgagtttt gataatttca aactcacatg ttctaacaca |
| 6001 | aaatttgctg atgatttaaa tcaaatgaca ggcttcacaa agccagcttc acgagagcta |
| 6061 | tctgtcacat tcttcccaga cttgaatggc gatgtagtgg ctattgacta tagacactat |
| 6121 | tcagcgagtt tcaagaaagg tgctaaatta ctgcataagc caattgtttg gcacattaac |
| 6181 | caggctacaa ccaagacaac gttcaaacca aacacttggt gtttacgttg tctttggagt |
| 6241 | acaaagccag tagatacttc aaattcattt gaagttctgg cagtagaaga cacacaagga |
| 6301 | atggacaatc ttgcttgtga aagtcaacaa cccacctctg aagaagtagt ggaaaatcct |
| 6361 | accatacaga aggaagtcat agagtgtgac gtgaaaacta ccgaagttgt aggcaatgtc |
| 6421 | atacttaaac catcagatga aggtgttaaa gtaacacaag agttaggtca tgaggatctt |
| 6481 | atggctgctt atgtggaaaa cacaagcatt accattaaga aacctaatga gctttcacta |
| 6541 | gccttaggtt taaaaacaat tgccactcat ggtattgctg caattaatag tgttccttgg |
| 6601 | agtaaaattt tggcttatgt caaaccattc ttaggacaag cagcaattac aacatcaaat |
| 6661 | tgcgctaaga gattagcaca acgtgtgttt aacaattata tgccttatgt gtttacatta |
| 6721 | ttgttccaat tgtgtacttt tactaaaagt accaattcta gaattagagc ttcactacct |
| 6781 | acaactattg ctaaaaatag tgttaagagt gttgctaaat tatgtttgga tgccggcatt |
| 6841 | aattatgtga agtcacccaa attttctaaa ttgttcacaa tcgctatgtg gctattgttg |
| 6901 | ttaagtattt gcttaggttc tctaatctgt gtaactgctg cttttggtgt actcttatct |
| 6961 | aattttggtg ctccttctta ttgtaatggc gttagagaat tgtatcttaa ttcgtctaac |
| 7021 | gttactacta tggatttctg tgaaggttct tttccttgca gcatttgttt aagtggatta |
| 7081 | gactcccttg attcttatcc agctcttgaa accattcagg tgacgatttc atcgtacaag |
| 7141 | ctagacttga caattttagg tctggccgct gagtgggttt tggcatatat gttgttcaca |
| 7201 | aaattctttt atttattagg tctttcagct ataatgcagg tgttctttgg ctattttgct |
| 7261 | agtcatttca tcagcaattc ttggctcatg tggtttatca ttagtattgt acaaatggca |
| 7321 | cccgtttctg caatggttag gatgtacatc ttctttgctt ctttctacta catatggaag |
| 7381 | agctatgttc atatcatgga tggttgcacc tcttcgactt gcatgatgtg ctataagcgc |
| 7441 | aatcgtgcca cacgcgttga gtgtacaact attgttaatg gcatgaagag atctttctat |
| 7501 | gtctatgcaa atggaggccg tggcttctgc aagactcaca attggaattg tctcaattgt |
| 7561 | gacacatttt gcactggtag tacattcatt agtgatgaag ttgctcgtga tttgtcactc |
| 7621 | cagtttaaaa gaccaatcaa ccctactgac cagtcatcgt atattgttga tagtgttgct |
| 7681 | gtgaaaaatg gcgcgcttca cctctacttt gacaaggctg gtcaaaagac ctatgagaga |
| 7741 | catccgctct cccattttgt caatttagac aatttgagag ctaacaacac taaaggttca |
| 7801 | ctgcctatta atgtcatagt ttttgatggc aagtccaaat gcgacgagtc tgcttctaag |
| 7861 | tctgcttctg tgtactacag tcagctgatg tgccaaccta ttctgttgct tgaccaagct |
| 7921 | cttgtatcag acgttggaga tagtactgaa gtttccgtta agatgtttga tgcttatgtc |
| 7981 | gacacctttt cagcaacttt tagtgttcct atggaaaaac ttaaggcact tgttgctaca |
| 8041 | gctcacagcg agttagcaaa gggtgtagct ttagatggtg tcctttctac attcgtgtca |
| 8101 | gctgcccgac aaggtgttgt tgataccgat gttgacacaa aggatgttat tgaatgtctc |
| 8161 | aaactttcac atcactctga cttagaagtg acaggtgaca gttgtaacaa tttcatgctc |
| 8221 | acctataata aggttgaaaa catgacgccc agagatcttg gcgcatgtat tgactgtaat |
| 8281 | gcaaggcata tcaatgccca agtagcaaaa agtcacaatg tttcactcat ctggaatgta |
| 8341 | aaagactaca tgtctttatc tgaacagctg cgtaaacaaa ttcgtagtgc tgccaagaag |
| 8401 | aacaacatac cttttagact aacttgtgct acaactagac aggttgtcaa tgtcataact |
| 8461 | actaaaatct cactcaaggg tggtaagatt gttagtactt gttttaaact tatgcttaag |
| 8521 | gccacattat tgtgcgttct tgctgcattg gtttgttata tcgttatgcc agtacataca |
| 8581 | ttgtcaatcc atgatggtta cacaaatgaa atcattggtt acaaagccat tcaggatggt |
| 8641 | gtcactcgtg acatcatttc tactgatgat tgttttgcaa ataaacatgc tggttttgac |
| 8701 | gcatggttta gccagcgtgg tggttcatac aaaaatgaca aaagctgccc tgtagtagct |
| 8761 | gctatcatta caagagagat tggtttcata gtgcctggct taccgggtac tgtgctgaga |
| 8821 | gcaatcaatg gtgacttctt gcattttcta cctcgtgttt ttagtgctgt tggcaacatt |
| 8881 | tgctacacac cttccaaact cattgagtat agtgattttg ctacctctgc ttgcgttctt |
| 8941 | gctgctgagt gtacaatttt taaggatgct atgggcaaac ctgtgccata ttgttatgac |
| 9001 | actaatttgc tagagggttc tatttcttat agtgagcttc gtccagacac tcgttatgtg |
| 9061 | cttatggatg gttccatcat acagtttcct aacacttacc tggagggttc tgttagagta |
| 9121 | gtaacaactt ttgatgctga gtactgtaga catggtacat gcgaaaggtc agaagtaggt |
| 9181 | atttgcctat ctaccagtgg tagatgggtt cttaataatg agcattacag agctctatca |
| 9241 | ggagttttct gtggtgttga tgcgatgaat ctcatagcta acatctttac tcctcttgtg |
| 9301 | caacctgtgg gtgctttaga tgtgtctgct tcagtagtgg ctggtggtat tattgccata |
| 9361 | ttggtgactt gtgctgccta ctactttatg aaattcagac gtgtttttgg tgagtacaac |
| 9421 | catgttgttg ctgctaatgc acttttgttt ttgatgtctt tcactatact ctgtctggta |
| 9481 | ccagcttaca gctttctgcc gggagtctac tcagtctttt acttgtactt gacattctat |
| 9541 | ttcaccaatg atgtttcatt cttggctcac cttcaatggt ttgccatgtt ttctcctatt |
| 9601 | gtgccttttt ggataacagc aatctatgta ttctgtattt ctctgaagca ctgccattgg |
| 9661 | ttctttaaca actatcttag gaaaagagtc atgtttaatg gagttacatt tagtaccttc |
| 9721 | gaggaggctg ctttgtgtac ctttttgctc aacaaggaaa tgtacctaaa attgcgtagc |
| 9781 | gagacactgt tgccacttac acagtataac aggtatcttg ctctatataa caagtacaag |
| 9841 | tatttcagtg gagccttaga tactaccagc tatcgtgaag cagcttgctg ccacttagca |
| 9901 | aaggctctaa atgactttag caactcaggt gctgatgttc tctaccaacc accacagaca |
| 9961 | tcaatcactt ctgctgttct gcagagtggt tttaggaaaa tggcattccc gtcaggcaaa |
| 10021 | gttgaagggt gcatggtaca agtaacctgt ggaactacaa ctcttaatgg attgtggttg |
| 10081 | gatgacacag tatactgtcc aagacatgtc atttgcacag cagaagacat gcttaatcct |
| 10141 | aactatgaag atctgctcat tcgcaaatcc aaccatagct ttcttgttca ggctggcaat |
| 10201 | gttcaacttc gtgttattgg ccattctatg caaaattgtc tgcttaggct taaagttgat |
| 10261 | acttctaacc ctaagacacc caagtataaa tttgtccgta tccaacctgg tcaaacattt |
| 10321 | tcagttctag catgctacaa tggttcacca tctggtgttt atcagtgtgc catgagacct |
| 10381 | aatcatacca ttaaaggttc tttccttaat ggatcatgtg gtagtgttgg ttttaacatt |
| 10441 | gattatgatt gcgtgtcttt ctgctatatg catcatatgg agcttccaac aggagtacac |
| 10501 | gctggtactg acttagaagg taaattctat ggtccatttg ttgacagaca aactgcacag |
| 10561 | gctgcaggta cagacacaac cataacatta aatgttttgg catggctgta tgctgctgtt |
| 10621 | atcaatggtg ataggtggtt tcttaataga ttcaccacta ctttgaatga ctttaacctt |
| 10681 | gtggcaatga agtacaacta tgaacctttg acacaagatc atgttgacat attgggacct |
| 10741 | ctttctgctc aaacaggaat tgccgtctta gatatgtgtg ctgctttgaa agagctgctg |
| 10801 | cagaatggta tgaatggtcg tactatcctt ggtagcacta ttttagaaga tgagtttaca |
| 10861 | ccatttgatg ttgttagaca atgctctggt gttaccttcc aaggtaagtt caagaaaatt |
| 10921 | gttaagggca ctcatcattg gatgctttta actttcttga catcactatt gattcttgtt |
| 10981 | caaagtacac agtggtcact gtttttcttt gtttacgaga atgctttctt gccatttact |
| 11041 | cttggtatta tggcaattgc tgcatgtgct atgctgcttg ttaagcataa gcacgcattc |
| 11101 | ttgtgcttgt ttctgttacc ttctcttgca acagttgctt actttaatat ggtctacatg |
| 11161 | cctgctagct gggtgatgcg tatcatgaca tggcttgaat tggctgacac tagcttgtct |
| 11221 | ggttataggc ttaaggattg tgttatgtat gcttcagctt tagttttgct tattctcatg |
| 11281 | acagctcgca ctgtttatga tgatgctgct agacgtgttt ggacactgat gaatgtcatt |
| 11341 | acacttgttt acaaagtcta ctatggtaat gctttagatc aagctatttc catgtgggcc |
| 11401 | ttagttattt ctgtaacctc taactattct ggtgtcgtta cgactatcat gtttttagct |
| 11461 | agagctatag tgtttgtgtg tgttgagtat tacccattgt tatttattac tggcaacacc |
| 11521 | ttacagtgta tcatgcttgt ttattgtttc ttaggctatt gttgctgctg ctactttggc |
| 11581 | cttttctgtt tactcaaccg ttacttcagg cttactcttg gtgtttatga ctacttggtc |
| 11641 | tctacacaag aatttaggta tatgaactcc caggggcttt tgcctcctaa gagtagtatt |
| 11701 | gatgctttca agcttaacat taagttgttg ggtattggag gtaaaccatg tatcaaggtt |
| 11761 | gctactgtac agtctaaaat gtctgacgta aagtgcacat ctgtggtact gctctcggtt |
| 11821 | cttcaacaac ttagagtaga gtcatcttct aaattgtggg cacaatgtgt acaactccac |
| 11881 | aatgatattc ttcttgcaaa agacacaact gaagctttcg agaagatggt ttctcttttg |
| 11941 | tctgttttgc tatccatgca gggtgctgta gacattaata ggttgtgcga ggaaatgctc |
| 12001 | gataaccgtg ctactcttca ggctattgct tcagaattta gttctttacc atcatatgcc |
| 12061 | gcttatgcca ctgcccagga ggcctatgag caggctgtag ctaatggtga ttctgaagtc |
| 12121 | gttctcaaaa agttaaagaa atctttgaat gtggctaaat ctgagtttga ccgtgatgct |
| 12181 | gccatgcaac gcaagttgga aaagatggca gatcaggcta tgacccaaat gtacaaacag |
| 12241 | gcaagatctg aggacaagag ggcaaaagta actagtgcta tgcaaacaat gctcttcact |
| 12301 | atgcttagga agcttgataa tgatgcactt aacaacatta tcaacaatgc gcgtgatggt |
| 12361 | tgtgttccac tcaacatcat accattgact acagcagcca aactcatggt tgttgtccct |
| 12421 | gattatggta cctacaagaa cacttgtgat ggtaacacct ttacatatgc atctgcactc |
| 12481 | tgggaaatcc agcaagttgt tgatgcggat agcaagattg ttcaacttag tgaaattaac |
| 12541 | atggacaatt caccaaattt ggcttggcct cttattgtta cagctctaag agccaactca |
| 12601 | gctgttaaac tacagaataa tgaactgagt ccagtagcac tacgacagat gtcctgtgcg |
| 12661 | gctggtacca cacaaacagc ttgtactgat gacaatgcac ttgcctacta taacaattcg |
| 12721 | aagggaggta ggtttgtgct ggcattacta tcagaccacc aagatctcaa atgggctaga |
| 12781 | ttccctaaga gtgatggtac aggtacaatt tacacagaac tggaaccacc ttgtaggttt |
| 12841 | gttacagaca caccaaaagg gcctaaagtg aaatacttgt acttcatcaa aggcttaaac |
| 12901 | aacctaaata gaggtatggt gctgggcagt ttagctgcta cagtacgtct tcaggctgga |
| 12961 | aatgctacag aagtacctgc caattcaact gtgctttcct tctgtgcttt tgcagtagac |
| 13021 | cctgctaaag catataagga ttacctagca agtggaggac aaccaatcac caactgtgtg |
| 13081 | aagatgttgt gtacacacac tggtacagga caggcaatta ctgtaacacc agaagctaac |
| 13141 | atggaccaag agtcctttgg tggtgcttca tgttgtctgt attgtagatg ccacattgac |
| 13201 | catccaaatc ctaaaggatt ctgtgacttg aaaggtaagt acgtccaaat acctaccact |
| 13261 | tgtgctaatg acccagtggg ttttacactt agaaacacag tctgtaccgt ctgcggaatg |
| 13321 | tggaaaggtt atggctgtag ttgtgaccaa ctccgcgaac ccttgatgca gtctgcggat |
| 13381 | gcatcaacgt ttttaaacgg gtttgcggtg taagtgcagc ccgtcttaca ccgtgcggca |
| 13441 | caggcactag tactgatgtc gtctacaggg cttttgatat ttacaacgaa aaagttgctg |
| 13501 | gttttgcaaa gttcctaaaa actaattgct gtcgcttcca ggagaaggat gaggaaggca |
| 13561 | atttattaga ctcttacttt gtagttaaga ggcatactat gtctaactac caacatgaag |
| 13621 | agactattta taacttggtt aaagattgtc cagcggttgc tgtccatgac tttttcaagt |
| 13681 | ttagagtaga tggtgacatg gtaccacata tatcacgtca gcgtctaact aaatacacaa |
| 13741 | tggctgattt agtctatgct ctacgtcatt ttgatgaggg taattgtgat acattaaaag |
| 13801 | aaatactcgt cacatacaat tgctgtgatg atgattattt caataagaag gattggtatg |
| 13861 | acttcgtaga gaatcctgac atcttacgcg tatatgctaa cttaggtgag cgtgtacgcc |
| 13921 | aatcattatt aaagactgta caattctgcg atgctatgcg tgatgcaggc attgtaggcg |
| 13981 | tactgacatt agataatcag gatcttaatg ggaactggta cgatttcggt gatttcgtac |
| 14041 | aagtagcacc aggctgcgga gttcctattg tggattcata ttactcattg ctgatgccca |
| 14101 | tcctcacttt gactagggca ttggctgctg agtcccatat ggatgctgat ctcgcaaaac |
| 14161 | cacttattaa gtgggatttg ctgaaatatg attttacgga agagagactt tgtctcttcg |
| 14221 | accgttattt taaatattgg gaccagacat accatcccaa ttgtattaac tgtttggatg |
| 14281 | ataggtgtat ccttcattgt gcaaacttta atgtgttatt ttctactgtg tttccaccta |
| 14341 | caagttttgg accactagta agaaaaatat ttgtagatgg tgttcctttt gttgtttcaa |
| 14401 | ctggatacca ttttcgtgag ttaggagtcg tacataatca ggatgtaaac ttacatagct |
| 14461 | cgcgtctcag tttcaaggaa cttttagtgt atgctgctga tccagctatg catgcagctt |
| 14521 | ctggcaattt attgctagat aaacgcacta catgcttttc agtagctgca ctaacaaaca |
| 14581 | atgttgcttt tcaaactgtc aaacccggta attttaataa agacttttat gactttgctg |
| 14641 | tgtctaaagg tttctttaag gaaggaagtt ctgttgaact aaaacacttc ttctttgctc |
| 14701 | aggatggcaa cgctgctatc agtgattatg actattatcg ttataatctg ccaacaatgt |
| 14761 | gtgatatcag acaactccta ttcgtagttg aagttgttga taaatacttt gattgttacg |
| 14821 | atggtggctg tattaatgcc aaccaagtaa tcgttaacaa tctggataaa tcagctggtt |
| 14881 | tcccatttaa taaatggggt aaggctagac tttattatga ctcaatgagt tatgaggatc |
| 14941 | aagatgcact tttcgcgtat actaagcgta atgtcatccc tactataact caaatgaatc |
| 15001 | ttaagtatgc cattagtgca aagaatagag ctcgcaccgt agctggtgtc tctatctgta |
| 15061 | gtactatgac aaatagacag tttcatcaga aattattgaa gtcaatagcc gccactagag |
| 15121 | gagctactgt ggtaattgga acaagcaagt tttacggtgg ctggcataat atgttaaaaa |
| 15181 | ctgtttacag tgatgtagaa actccacacc ttatgggttg ggattatcca aaatgtgaca |
| 15241 | gagccatgcc taacatgctt aggataatgg cctctcttgt tcttgctcgc aaacataaca |
| 15301 | cttgctgtaa cttatcacac cgtttctaca ggttagctaa cgagtgtgcg caagtattaa |
| 15361 | gtgagatggt catgtgtggc ggctcactat atgttaaacc aggtggaaca tcatccggtg |
| 15421 | atgctacaac tgcttatgct aatagtgtct ttaacatttg tcaagctgtt acagccaatg |
| 15481 | taaatgcact tctttcaact gatggtaata agatagctga caagtatgtc cgcaatctac |
| 15541 | aacacaggct ctatgagtgt ctctatagaa atagggatgt tgatcatgaa ttcgtggatg |
| 15601 | agttttacgc ttacctgcgt aaacatttct ccatgatgat tctttctgat gatgccgttg |
| 15661 | tgtgctataa cagtaactat gcggctcaag gtttagtagc tagcattaag aactttaagg |
| 15721 | cagttcttta ttatcaaaat aatgtgttca tgtctgaggc aaaatgttgg actgagactg |
| 15781 | accttactaa aggacctcac gaattttgct cacagcatac aatgctagtt aaacaaggag |
| 15841 | atgattacgt gtacctgcct tacccagatc catcaagaat attaggcgca ggctgttttg |
| 15901 | tcgatgatat tgtcaaaaca gatggtacac ttatgattga aaggttcgtg tcactggcta |
| 15961 | ttgatgctta cccacttaca aaacatccta atcaggagta tgctgatgtc tttcacttgt |
| 16021 | atttacaata cattagaaag ttacatgatg agcttactgg ccacatgttg gacatgtatt |
| 16081 | ccgtaatgct aactaatgat aacacctcac ggtactggga acctgagttt tatgaggcta |
| 16141 | tgtacacacc acatacagtc ttgcaggctg taggtgcttg tgtattgtgc aattcacaga |
| 16201 | cttcacttcg ttgcggtgcc tgtattagga gaccattcct atgttgcaag tgctgctatg |
| 16261 | accatgtcat ttcaacatca cacaaattag tgttgtctgt taatccctat gtttgcaatg |
| 16321 | ccccaggttg tgatgtcact gatgtgacac aactgtatct aggaggtatg agctattatt |
| 16381 | gcaagtcaca taagcctccc attagttttc cattatgtgc taatggtcag gtttttggtt |
| 16441 | tatacaaaaa cacatgtgta ggcagtgaca atgtcactga cttcaatgcg atagcaacat |
| 16501 | gtgattggac taatgctggc gattacatac ttgccaacac ttgtactgag agactcaagc |
| 16561 | ttttcgcagc agaaacgctc aaagccactg aggaaacatt taagctgtca tatggtattg |
| 16621 | ccactgtacg cgaagtactc tctgacagag aattgcatct ttcatgggag gttggaaaac |
| 16681 | ctagaccacc attgaacaga aactatgtct ttactggtta ccgtgtaact aaaaatagta |
| 16741 | aagtacagat tggagagtac acctttgaaa aaggtgacta tggtgatgct gttgtgtaca |
| 16801 | gaggtactac gacatacaag ttgaatgttg gtgattactt tgtgttgaca tctcacactg |
| 16861 | taatgccact tagtgcacct actctagtgc cacaagagca ctatgtgaga attactggct |
| 16921 | tgtacccaac actcaacatc tcagatgagt tttctagcaa tgttgcaaat tatcaaaagg |
| 16981 | tcggcatgca aaagtactct acactccaag gaccacctgg tactggtaag agtcattttg |
| 17041 | ccatcggact tgctctctat tacccatctg ctcgcatagt gtatacggca tgctctcatg |
| 17101 | cagctgttga tgccctatgt gaaaaggcat taaaatattt gcccatagat aaatgtagta |
| 17161 | gaatcatacc tgcgcgtgcg cgcgtagagt gttttgataa attcaaagtg aattcaacac |
| 17221 | tagaacagta tgttttctgc actgtaaatg cattgccaga aacaactgct gacattgtag |
| 17281 | tctttgatga aatctctatg gctactaatt atgacttgag tgttgtcaat gctagacttc |
| 17341 | gtgcaaaaca ctacgtctat attggcgatc ctgctcaatt accagccccc cgcacattgc |
| 17401 | tgactaaagg cacactagaa ccagaatatt ttaattcagt gtgcagactt atgaaaacaa |
| 17461 | taggtccaga catgttcctt ggaacttgtc gccgttgtcc tgctgaaatt gttgacactg |
| 17521 | tgagtgcttt agtttatgac aataagctaa aagcacacaa ggataagtca gctcaatgct |
| 17581 | tcaaaatgtt ctacaaaggt gttattacac atgatgtttc atctgcaatc aacagacctc |
| 17641 | aaataggcgt tgtaagagaa tttcttacac gcaatcctgc ttggagaaaa gctgttttta |
| 17701 | tctcacctta taattcacag aacgctgtag cttcaaaaat cttaggattg cctacgcaga |
| 17761 | ctgttgattc atcacagggt tctgaatatg actatgtcat attcacacaa actactgaaa |
| 17821 | cagcacactc ttgtaatgtc aaccgcttca atgtggctat cacaagggca aaaattggca |
| 17881 | ttttgtgcat aatgtctgat agagatcttt atgacaaact gcaatttaca agtctagaaa |
| 17941 | taccacgtcg caatgtggct acattacaag cagaaaatgt aactggactt tttaaggact |
| 18001 | gtagtaagat cattactggt cttcatccta cacaggcacc tacacacctc agcgttgata |
| 18061 | taaagttcaa gactgaagga ttatgtgttg acataccagg cataccaaag gacatgacct |
| 18121 | accgtagact catctctatg atgggtttca aaatgaatta ccaagtcaat ggttacccta |
| 18181 | atatgtttat cacccgcgaa gaagctattc gtcacgttcg tgcgtggatt ggctttgatg |
| 18241 | tagagggctg tcatgcaact agagatgctg tgggtactaa cctacctctc cagctaggat |
| 18301 | tttctacagg tgttaactta gtagctgtac cgactggtta tgttgacact gaaaataaca |
| 18361 | cagaattcac cagagttaat gcaaaacctc caccaggtga ccagtttaaa catcttatac |
| 18421 | cactcatgta taaaggcttg ccctggaatg tagtgcgtat taagatagta caaatgctca |
| 18481 | gtgatacact gaaaggattg tcagacagag tcgtgttcgt cctttgggcg catggctttg |
| 18541 | agcttacatc aatgaagtac tttgtcaaga ttggacctga aagaacgtgt tgtctgtgtg |
| 18601 | acaaacgtgc aacttgcttt tctacttcat cagatactta tgcctgctgg aatcattctg |
| 18661 | tgggttttga ctatgtctat aacccattta tgattgatgt tcagcagtgg ggctttacgg |
| 18721 | gtaaccttca gagtaaccat gaccaacatt gccaggtaca tggaaatgca catgtggcta |
| 18781 | gttgtgatgc tatcatgact agatgtttag cagtccatga gtgctttgtt aagcgcgttg |
| 18841 | attggtctgt tgaataccct attataggag atgaactgag ggttaattct gcttgcagaa |
| 18901 | aagtacaaca catggttgtg aagtctgcat tgcttgctga taagtttcca gttcttcatg |
| 18961 | acattggaaa tccaaaggct atcaagtgtg tgcctcaggc tgaagtagaa tggaagttct |
| 19021 | acgatgctca gccatgtagt gacaaagctt acaaaataga ggaactcttc tattcttatg |
| 19081 | ctacacatca cgataaattc actgatggtg tttgtttgtt ttggaattgt aacgttgatc |
| 19141 | gttacccagc caatgcaatt gtgtgtaggt ttgacacaag agtcttgtca aacttgaact |
| 19201 | taccaggctg tgatggtggt agtttgtatg tgaataagca tgcattccac actccagctt |
| 19261 | tcgataaaag tgcatttact aatttaaagc aattgccttt cttttactat tctgatagtc |
| 19321 | cttgtgagtc tcatggcaaa caagtagtgt cggatattga ttatgttcca ctcaaatctg |
| 19381 | ctacgtgtat tacacgatgc aatttaggtg gtgctgtttg cagacaccat gcaaatgagt |
| 19441 | accgacagta cttggatgca tataatatga tgatttctgc tggatttagc ctatggattt |
| 19501 | acaaacaatt tgatacttat aacctgtgga atacatttac caggttacag agtttagaaa |
| 19561 | atgtggctta taatgttgtt aataaaggac actttgatgg acacgccggc gaagcacctg |
| 19621 | tttccatcat taataatgct gtttacacaa aggtagatgg tattgatgtg gagatctttg |
| 19681 | aaaataagac aacacttcct gttaatgttg catttgagct ttgggctaag cgtaacatta |
| 19741 | aaccagtgcc agagattaag atactcaata atttgggtgt tgatatcgct gctaatactg |
| 19801 | taatctggga ctacaaaaga gaagccccag cacatgtatc tacaataggt gtctgcacaa |
| 19861 | tgactgacat tgccaagaaa cctactgaga gtgcttgttc ttcacttact gtcttgtttg |
| 19921 | atggtagagt ggaaggacag gtagaccttt ttagaaacgc ccgtaatggt gttttaataa |
| 19981 | cagaaggttc agtcaaaggt ctaacacctt caaagggacc agcacaagct agcgtcaatg |
| 20041 | gagtcacatt aattggagaa tcagtaaaaa cacagtttaa ctactttaag aaagtagacg |
| 20101 | gcattattca acagttgcct gaaacctact ttactcagag cagagactta gaggatttta |
| 20161 | agcccagatc acaaatggaa actgactttc tcgagctcgc tatggatgaa ttcatacagc |
| 20221 | gatataagct cgagggctat gccttcgaac acatcgttta tggagatttc agtcatggac |
| 20281 | aacttggcgg tcttcattta atgataggct tagccaagcg ctcacaagat tcaccactta |
| 20341 | aattagagga ttttatccct atggacagca cagtgaaaaa ttacttcata acagatgcgc |
| 20401 | aaacaggttc atcaaaatgt gtgtgttctg tgattgatct tttacttgat gactttgtcg |
| 20461 | agataataaa gtcacaagat ttgtcagtga tttcaaaagt ggtcaaggtt acaattgact |
| 20521 | atgctgaaat ttcattcatg ctttggtgta aggatggaca tgttgaaacc ttctacccaa |
| 20581 | aactacaagc aagtcaagcg tggcaaccag gtgttgcgat gcctaacttg tacaagatgc |
| 20641 | aaagaatgct tcttgaaaag tgtgaccttc agaattatgg tgaaaatgct gttataccaa |
| 20701 | aaggaataat gatgaatgtc gcaaagtata ctcaactgtg tcaatactta aatacactta |
| 20761 | ctttagctgt accctacaac atgagagtta ttcactttgg tgctggctct gataaaggag |
| 20821 | ttgcaccagg tacagctgtg ctcagacaat ggttgccaac tggcacacta cttgtcgatt |
| 20881 | cagatcttaa tgacttcgtc tccgacgcag attctacttt aattggagac tgtgcaacag |
| 20941 | tacatacggc taataaatgg gaccttatta ttagcgatat gtatgaccct aggaccaaac |
| 21001 | atgtgacaaa agagaatgac tctaaagaag ggtttttcac ttatctgtgt ggatttataa |
| 21061 | agcaaaaact agccctgggt ggttctatag ctgtaaagat aacagagcat tcttggaatg |
| 21121 | ctgaccttta caagcttatg ggccatttct catggtggac agcttttgtt acaaatgtaa |
| 21181 | atgcatcatc atcggaagca tttttaattg gggctaacta tcttggcaag ccgaaggaac |
| 21241 | aaattgatgg ctataccatg catgctaact acattttctg gaggaacaca aatcctatcc |
| 21301 | agttgtcttc ctattcactc tttgacatga gcaaatttcc tcttaaatta agaggaactg |
| 21361 | ctgtaatgtc tcttaaggag aatcaaatca atgatatgat ttattctctt ctggaaaaag |
| 21421 | gtaggcttat cattagagaa aacaacagag ttgtggtttc aagtgatatt cttgttaaca |
| 21481 | actaaacgaa catgtttatt ttcttattat ttcttactct cactagtggt agtgaccttg |
| 21541 | accggtgcac cacttttgat gatgttcaag ctcctaatta cactcaacat acttcatcta |
| 21601 | tgaggggggt ttactatcct gatgaaattt ttagatcaga cactctttat ttaactcagg |
| 21661 | atttatttct tccattttat tctaatgtta cagggtttca tactattaat catacgtttg |
| 21721 | gcaaccctgt catacctttt aaggatggta tttattttgc tgccacagag aaatcaaatg |
| 21781 | ttgtccgtgg ttgggttttt ggttctacca tgaacaacaa gtcacagtcg gtgattatta |
| 21841 | ttaacaattc tactaatgtt gttatacgag catgtaactt tgaattgtgt gacaaccctt |
| 21901 | tctttgctgt ttctaaaccc atgggtacac agacacatac tatgatattc gataatgcat |
| 21961 | ttaattgcac tttcgagtac atatctgatg ccttttcgct tgatgtttca gaaaagtcag |
| 22021 | gtaattttaa acacttacga gagtttgtgt ttaaaaataa agatgggttt ctctatgttt |
| 22081 | ataagggcta tcaacctata gatgtagttc gtgatctacc ttctggtttt aacactttga |
| 22141 | aacctatttt taagttgcct cttggtatta acattacaaa ttttagagcc attcttacag |
| 22201 | ccttttcacc tgctcaagac atttggggca cgtcagctgc agcctatttt gttggctatt |
| 22261 | taaagccaac tacatttatg ctcaagtatg atgaaaatgg tacaatcaca gatgctgttg |
| 22321 | attgttctca aaatccactt gctgaactca aatgctctgt taagagcttt gagattgaca |
| 22381 | aaggaattta ccagacctct aatttcaggg ttgttccctc aggagatgtt gtgagattcc |
| 22441 | ctaatattac aaacttgtgt ccttttggag aggtttttaa tgctactaaa ttcccttctg |
| 22501 | tctatgcatg ggagagaaaa aaaatttcta attgtgttgc tgattactct gtgctctaca |
| 22561 | actcaacatt tttttcaacc tttaagtgct atggcgtttc tgccactaag ttgaatgatc |
| 22621 | tttgcttctc caatgtctat gcagattctt ttgtagtcaa gggagatgat gtaagacaaa |
| 22681 | tagcgccagg acaaactggt gttattgctg attataatta taaattgcca gatgatttca |
| 22741 | tgggttgtgt ccttgcttgg aatactagga acattgatgc tacttcaact ggtaattata |
| 22801 | attataaata taggtatctt agacatggca agcttaggcc ctttgagaga gacatatcta |
| 22861 | atgtgccttt ctcccctgat ggcaaacctt gcaccccacc tgctcttaat tgttattggc |
| 22921 | cattaaatga ttatggtttt tacaccacta ctggcattgg ctaccaacct tacagagttg |
| 22981 | tagtactttc ttttgaactt ttaaatgcac cggccacggt ttgtggacca aaattatcca |
| 23041 | ctgaccttat taagaaccag tgtgtcaatt ttaattttaa tggactcact ggtactggtg |
| 23101 | tgttaactcc ttcttcaaag agatttcaac catttcaaca atttggccgt gatgtttctg |
| 23161 | atttcactga ttccgttcga gatcctaaaa catctgaaat attagacatt tcaccttgcg |
| 23221 | cttttggggg tgtaagtgta attacacctg gaacaaatgc ttcatctgaa gttgctgttc |
| 23281 | tatatcaaga tgttaactgc actgatgttt ctacagcaat tcatgcagat caactcacac |
| 23341 | cagcttggcg catatattct actggaaaca atgtattcca gactcaagca ggctgtctta |
| 23401 | taggagctga gcatgtcgac acttcttatg agtgcgacat tcctattgga gctggcattt |
| 23461 | gtgctagtta ccatacagtt tctttattac gtagtactag ccaaaaatct attgtggctt |
| 23521 | atactatgtc tttaggtgct gatagttcaa ttgcttactc taataacacc attgctatac |
| 23581 | ctactaactt ttcaattagc attactacag aagtaatgcc tgtttctatg gctaaaacct |
| 23641 | ccgtagattg taatatgtac atctgcggag attctactga atgtgctaat ttgcttctcc |
| 23701 | aatatggtag cttttgcaca caactaaatc gtgcactctc aggtattgct gctgaacagg |
| 23761 | atcgcaacac acgtgaagtg ttcgctcaag tcaaacaaat gtacaaaacc ccaactttga |
| 23821 | aatattttgg tggttttaat ttttcacaaa tattacctga ccctctaaag ccaactaaga |
| 23881 | ggtcttttat tgaggacttg ctctttaata aggtgacact cgctgatgct ggcttcatga |
| 23941 | agcaatatgg cgaatgccta ggtgatatta atgctagaga tctcatttgt gcgcagaagt |
| 24001 | tcaatggact tacagtgttg ccacctctgc tcactgatga tatgattgct gcctacactg |
| 24061 | ctgctctagt tagtggtact gccactgctg gatggacatt tggtgctggc gctgctcttc |
| 24121 | aaataccttt tgctatgcaa atggcatata ggttcaatgg cattggagtt acccaaaatg |
| 24181 | ttctctatga gaaccaaaaa caaatcgcca accaatttaa caaggcgatt agtcaaattc |
| 24241 | aagaatcact tacaacaaca tcaactgcat tgggcaagct gcaagacgtt gttaaccaga |
| 24301 | atgctcaagc attaaacaca cttgttaaac aacttagctc taattttggt gcaatttcaa |
| 24361 | gtgtgctaaa tgatatcctt tcgcgacttg ataaagtcga ggcggaggta caaattgaca |
| 24421 | ggttaattac aggcagactt caaagccttc aaacctatgt aacacaacaa ctaatcaggg |
| 24481 | ctgctgaaat cagggcttct gctaatcttg ctgctactaa aatgtctgag tgtgttcttg |
| 24541 | gacaatcaaa aagagttgac ttttgtggaa agggctacca ccttatgtcc ttcccacaag |
| 24601 | cagccccgca tggtgttgtc ttcctacatg tcacgtatgt gccatcccag gagaggaact |
| 24661 | tcaccacagc gccagcaatt tgtcatgaag gcaaagcata cttccctcgt gaaggtgttt |
| 24721 | ttgtgtttaa tggcacttct tggtttatta cacagaggaa cttcttttct ccacaaataa |
| 24781 | ttactacaga caatacattt gtctcaggaa attgtgatgt cgttattggc atcattaaca |
| 24841 | acacagttta tgatcctctg caacctgagc ttgactcatt caaagaagag ctggacaagt |
| 24901 | acttcaaaaa tcatacatca ccagatgttg atcttggcga catttcaggc attaacgctt |
| 24961 | ctgtcgtcaa cattcaaaaa gaaattgacc gcctcaatga ggtcgctaaa aatttaaatg |
| 25021 | aatcactcat tgaccttcaa gaattgggaa aatatgagca atatattaaa tggccttggt |
| 25081 | atgtttggct cggcttcatt gctggactaa ttgccatcgt catggttaca atcttgcttt |
| 25141 | gttgcatgac tagttgttgc agttgcctca agggtgcatg ctcttgtggt tcttgctgca |
| 25201 | agtttgatga ggatgactct gagccagttc tcaagggtgt caaattacat tacacataaa |
| 25261 | cgaacttatg gatttgttta tgagattttt tactcttaga tcaattactg cacagccagt |
| 25321 | aaaaattgac aatgcttctc ctgcaagtac tgttcatgct acagcaacga taccgctaca |
| 25381 | agcctcactc cctttcggat ggcttgttat tggcgttgca tttcttgctg tttttcagag |
| 25441 | cgctaccaaa ataattgcgc tcaataaaag atggcagcta gccctttata agggcttcca |
| 25501 | gttcatttgc aatttactgc tgctatttgt taccatctat tcacatcttt tgcttgtcgc |
| 25561 | tgcaggtatg gaggcgcaat ttttgtacct ctatgccttg atatattttc tacaatgcat |
| 25621 | caacgcatgt agaattatta tgagatgttg gctttgttgg aagtgcaaat ccaagaaccc |
| 25681 | attactttat gatgccaact actttgtttg ctggcacaca cataactatg actactgtat |
| 25741 | accatataac agtgtcacag atacaattgt cgttactgaa ggtgacggca tttcaacacc |
| 25801 | aaaactcaaa gaagactacc aaattggtgg ttattctgag gataggcact caggtgttaa |
| 25861 | agactatgtc gttgtacatg gctatttcac cgaagtttac taccagcttg agtctacaca |
| 25921 | aattactaca gacactggta ttgaaaatgc tacattcttc atctttaaca agcttgttaa |
| 25981 | agacccaccg aatgtgcaaa tacacacaat cgacggctct tcaggagttg ctaatccagc |
| 26041 | aatggatcca atttatgatg agccgacgac gactactagc gtgcctttgt aagcacaaga |
| 26101 | aagtgagtac gaacttatgt actcattcgt ttcggaagaa acaggtacgt taatagttaa |
| 26161 | tagcgtactt ctttttcttg ctttcgtggt attcttgcta gtcacactag ccatccttac |
| 26221 | tgcgcttcga ttgtgtgcgt actgctgcaa tattgttaac gtgagtttag taaaaccaac |
| 26281 | ggtttacgtc tactcgcgtg ttaaaaatct gaactcttct gaaggagttc ctgatcttct |
| 26341 | ggtctaaacg aactaactat tattattatt ctgtttggaa ctttaacatt gcttatcatg |
| 26401 | gcagacaacg gtactattac cgttgaggag cttaaacaac tcctggaaca atggaaccta |
| 26461 | gtaataggtt tcctattcct agcctggatt atgttactac aatttgccta ttctaatcgg |
| 26521 | aacaggtttt tgtacataat aaagcttgtt ttcctctggc tcttgtggcc agtaacactt |
| 26581 | gcttgttttg tgcttgctgc tgtctacaga attaattggg tgactggcgg gattgcgatt |
| 26641 | gcaatggctt gtattgtagg cttgatgtgg cttagctact tcgttgcttc cttcaggctg |
| 26701 | tttgctcgta cccgctcaat gtggtcattc aacccagaaa caaacattct tctcaatgtg |
| 26761 | cctctccggg ggacaattgt gaccagaccg ctcatggaaa gtgaacttgt cattggtgct |
| 26821 | gtgatcattc gtggtcactt gcgaatggcc ggacactccc tagggcgctg tgacattaag |
| 26881 | gacctgccaa aagagatcac tgtggctaca tcacgaacgc tttcttatta caaattagga |
| 26941 | gcgtcgcagc gtgtaggcac tgattcaggt tttgctgcat acaaccgcta ccgtattgga |
| 27001 | aactataaat taaatacaga ccacgccggt agcaacgaca atattgcttt gctagtacag |
| 27061 | taagtgacaa cagatgtttc atcttgttga cttccaggtt acaatagcag agatattgat |
| 27121 | tatcattatg aggactttca ggattgctat ttggaatctt gacgttataa taagttcaat |
| 27181 | agtgagacaa ttatttaagc ctctaactaa gaagaattat tcggagttag atgatgaaga |
| 27241 | acctatggag ttagattatc cataaaacga acatgaaaat tattctcttc ctgacattga |
| 27301 | ttgtatttac atcttgcgag ctatatcact atcaggagtg tgttagaggt acgactgtac |
| 27361 | tactaaaaga accttgccca tcaggaacat acgagggcaa ttcaccattt caccctcttg |
| 27421 | ctgacaataa atttgcacta acttgcacta gcacacactt tgcttttgct tgtgctgacg |
| 27481 | gtactcgaca tacctatcag ctgcgtgcaa gatcagtttc accaaaactt ttcatcagac |
| 27541 | aagaggaggt tcaacaagag ctctactcgc cactttttct cattgttgct gctctagtat |
| 27601 | ttttaatact ttgcttcacc attaagagaa agacagaatg aatgagctca ctttaattga |
| 27661 | cttctatttg tgctttttag cctttctgct attccttgtt ttaataatgc ttattatatt |
| 27721 | ttggttttca ctcgaaatcc aggatctaga agaaccttgt accaaagtct aaacgaacat |
| 27781 | gaaacttctc attgttttga cttgtatttc tctatgcagt tgcatatgca ctgtagtaca |
| 27841 | gcgctgtgca tctaataaac ctcatgtgct tgaagatcct tgtaaggtac aacactaggg |
| 27901 | gtaatactta tagcactgct tggctttgtg ctctaggaaa ggttttacct tttcatagat |
| 27961 | ggcacactat ggttcaaaca tgcacaccta atgttactat caactgtcaa gatccagctg |
| 28021 | gtggtgcgct tatagctagg tgttggtacc ttcatgaagg tcaccaaact gctgcattta |
| 28081 | gagacgtact tgttgtttta aataaacgaa caaattaaaa tgtctgataa tggaccccaa |
| 28141 | tcaaaccaac gtagtgcccc ccgcattaca tttggtggac ccacagattc aactgacaat |
| 28201 | aaccagaatg gaggacgcaa tggggcaagg ccaaaacagc gccgacccca aggtttaccc |
| 28261 | aataatactg cgtcttggtt cacagctctc actcagcatg gcaaggagga acttagattc |
| 28321 | cctcgaggcc agggcgttcc aatcaacacc aatagtggtc cagatgacca aattggctac |
| 28381 | taccgaagag ctacccgacg agttcgtggt ggtgacggca aaatgaaaga gctcagcccc |
| 28441 | agatggtact tctattacct aggaactggc ccagaagctt cacttcccta cggcgctaac |
| 28501 | aaagaaggca tcgtatgggt tgcaactgag ggagccttga atacacccaa agaccacatt |
| 28561 | ggcacccgca atcctaataa caatgctgcc accgtgctac aacttcctca aggaacaaca |
| 28621 | ttgccaaaag gcttctacgc agagggaagc agaggcggca gtcaagcctc ttctcgctcc |
| 28681 | tcatcacgta gtcgcggtaa ttcaagaaat tcaactcctg gcagcagtag gggaaattct |
| 28741 | cctgctcgaa tggctagcgg aggtggtgaa actgccctcg cgctattgct gctagacaga |
| 28801 | ttgaaccagc ttgagagcaa agtttctggt aaaggccaac aacaacaagg ccaaactgtc |
| 28861 | actaagaaat ctgctgctga ggcatctaaa aagcctcgcc aaaaacgtac tgccacaaaa |
| 28921 | cagtacaacg tcactcaagc atttgggaga cgtggtccag aacaaaccca aggaaatttc |
| 28981 | ggggaccaag acctaatcag acaaggaact gattacaaac attggccgca aattgcacaa |
| 29041 | tttgctccaa gtgcctctgc attctttgga atgtcacgca ttggcatgga agtcacacct |
| 29101 | tcgggaacat ggctgactta tcatggagcc attaaattgg atgacaaaga tccacaattc |
| 29161 | aaagacaacg tcatactgct gaacaagcac attgacgcat acaaaacatt cccaccaaca |
| 29221 | gagcctaaaa aggacaaaaa gaaaaagact gatgaagctc agcctttgcc gcagagacaa |
| 29281 | aagaagcagc ccactgtgac tcttcttcct gcggctgaca tggatgattt ctccagacaa |
| 29341 | cttcaaaatt ccatgagtgg agcttctgct gattcaactc aggcataaac actcatgatg |
| 29401 | accacacaag gcagatgggc tatgtaaacg ttttcgcaat tccgtttacg atacatagtc |
| 29461 | tactcttgtg cagaatgaat tctcgtaact aaacagcaca agtaggttta gttaacttta |
| 29521 | atctcacata gcaatcttta atcaatgtgt aacattaggg aggacttgaa agagccacca |
| 29581 | cattttcatc gaggccacgc ggagtacgat cgagggtaca gtgaataatg ctagggagag |
| 29641 | ctgcctatat ggaagagccc taatgtgtaa aattaatttt agtagtgcta tccccatgtg |
| 29701 | attttaatag cttcttagga gaatgacaaa aaaaaaaaaa aaaaaaaaaa a. |

GenBank Accession No. GenBank: JX869059.2, which is specifically incorporated by reference herein in its entirety, provides the following (DNA) genomic sequence for MERS-CoV (Human betacoronavirus 2c EMC/2012, complete genome):

| (SEQ ID NO: 4) | |
|---|---|
| 1 | gatttaagtg aatagcttgg ctatctcact tcccctcgtt ctcttgcaga actttgattt |
| 61 | taacgaactt aaataaaagc cctgttgttt agcgtatcgt tgcacttgtc tggtgggatt |
| 121 | gtggcattaa tttgcctgct catctaggca gtggacatat gctcaacact gggtataatt |
| 181 | ctaattgaat actatttttc agttagagcg tcgtgtctct tgtacgtctc ggtcacaata |
| 241 | cacggtttcg tccggtgcgt ggcaattcgg ggcacatcat gtctttcgtg gctggtgtga |
| 301 | ccgcgcaagg tgcgcgcggt acgtatcgag cagcgctcaa ctctgaaaaa catcaagacc |
| 361 | atgtgtctct aactgtgcca ctctgtggtt caggaaacct ggttgaaaaa ctttcaccat |
| 421 | ggttcatgga tggcgaaaat gcctatgaag tggtgaaggc catgttactt aaaaaggagc |
| 481 | cacttctcta tgtgcccatc cggctggctg gacacactag acacctccca ggtcctcgtg |
| 541 | tgtacctggt tgagaggctc attgcttgtg aaaatccatt catggttaac caattggctt |
| 601 | atagctctag tgcaaatggc agcctggttg gcacaacttt gcagggcaag cctattggta |
| 661 | tgttcttccc ttatgacatc gaacttgtca caggaaagca aaatattctc ctgcgcaagt |
| 721 | atggccgtgg tggttatcac tacaccccat tccactatga gcgagacaac acctcttgcc |
| 781 | ctgagtggat ggacgatttt gaggcggatc ctaaaggcaa atatgcccag aatctgctta |
| 841 | agaagttgat tggcggtgat gtcactccag ttgaccaata catgtgtggc gttgatggaa |
| 901 | aacccattag tgcctacgca tttttaatgg ccaaggatgg aataaccaaa ctggctgatg |
| 961 | ttgaagcgga cgtcgcagca cgtgctgatg acgaaggctt catcacatta aagaacaatc |
| 1021 | tatatagatt ggtttggcat gttgagcgta aagacgttcc atatcctaag caatctattt |
| 1081 | ttactattaa tagtgtggtc caaaaggatg gtgttgaaaa cactcctcct cactatttta |
| 1141 | ctcttggatg caaaatttta acgctcaccc cacgcaacaa gtggagtggc gtttctgact |
| 1201 | tgtccctcaa acaaaaactc ctttacacct tctatggtaa ggagtcactt gagaacccaa |
| 1261 | cctacattta ccactccgca ttcattgagt gtggaagttg tggtaatgat tcctggctta |
| 1321 | cagggaatgc tatccaaggg tttgcctgtg gatgtggggc atcatataca gctaatgatg |
| 1381 | tcgaagtcca atcatctggc atgattaagc caaatgctct tctttgtgct acttgcccct |
| 1441 | ttgctaaggg tgatagctgt tcttctaatt gcaaacattc agttgctcag ttggttagtt |
| 1501 | acctttctga acgctgtaat gttattgctg attctaagtc cttcacactt atctttggtg |
| 1561 | gcgtagctta cgcctacttt ggatgtgagg aaggtactat gtactttgtg cctagagcta |
| 1621 | agtctgttgt ctcaaggatt ggagactcca tctttacagg ctgtactggc tcttggaaca |
| 1681 | aggtcactca aattgctaac atgttcttgg aacagactca gcattccctt aactttgtgg |
| 1741 | gagagttcgt tgtcaacgat gttgtcctcg caattctctc tggaaccaca actaatgttg |
| 1801 | acaaaatacg ccagcttctc aaaggtgtca cccttgacaa gttgcgtgat tatttagctg |
| 1861 | actatgacgt agcagtcact gccggcccat tcatggataa tgctattaat gttggtggta |
| 1921 | caggattaca gtatgccgcc attactgcac cttatgtagt tctcactggc ttaggtgagt |
| 1981 | cctttaagaa agttgcaacc ataccgtata aggtttgcaa ctctgttaag gatactctgg |
| 2041 | cttattatgc tcacagcgtg ttgtacagag tttttcctta tgacatggat tctggtgtgt |
| 2101 | catcctttag tgaactactt tttgattgcg ttgatctttc agtagcttct acctattttt |
| 2161 | tagtccgcat cttgcaagat aagactggcg actttatgtc tacaattatt acttcctgcc |
| 2221 | aaactgctgt tagtaagctt ctagatacat gttttgaagc tacagaagca acatttaact |
| 2281 | tcttgttaga tttggcagga ttgttcagaa tctttctccg caatgcctat gtgtacactt |
| 2341 | cacaagggtt tgtggtggtc aatggcaaag tttctacact tgtcaaacaa gtgttagact |
| 2401 | tgcttaataa gggtatgcaa cttttgcata caaaggtctc ctgggctggt tctaaaatca |
| 2461 | ttgctgttat ctacagcggc agggagtctc taatattccc atcgggaacc tattactgtg |
| 2521 | tcaccactaa ggctaagtcc gttcaacaag atcttgacgt tattttgcct ggtgagtttt |
| 2581 | ccaagaagca gttaggactg ctccaaccta ctgacaattc tacaactgtt agtgttactg |
| 2641 | tatccagtaa catggttgaa actgttgtgg gtcaacttga gcaaactaat atgcatagtc |
| 2701 | ctgatgttat agtaggtgac tatgtcatta ttagtgaaaa attgtttgtg cgtagtaagg |
| 2761 | aagaagacgg atttgccttc taccctgctt gcactaatgg tcatgctgta ccgactctct |
| 2821 | ttagacttaa gggaggtgca cctgtaaaaa aagtagcctt tggcggtgat caagtacatg |
| 2881 | aggttgctgc tgtaagaagt gttactgtcg agtacaacat tcatgctgta ttagacacac |
| 2941 | tacttgcttc ttctagtctt agaacctttg ttgtagataa gtctttgtca attgaggagt |
| 3001 | ttgctgacgt agtaaaggaa caagtctcag acttgcttgt taaattactg cgtggaatgc |
| 3061 | cgattccaga ttttgattta gacgatttta ttgacgcacc atgctattgc tttaacgctg |
| 3121 | agggtgatgc atcctggtct tctactatga tcttctctct tcaccccgtc gagtgtgacg |
| 3181 | aggagtgttc tgaagtagag gcttcagatt tagaagaagg tgaatcagag tgcatttctg |
| 3241 | agacttcaac tgaacaagtt gacgtttctc atgagacttc tgacgacgag tgggctgctg |
| 3301 | cagttgatga agcgttccct ctcgatgaag cagaagatgt tactgaatct gtgcaagaag |
| 3361 | aagcacaacc agtagaagta cctgttgaag atattgcgca ggttgtcata gctgacacct |
| 3421 | tacaggaaac tcctgttgtg cctgatactg ttgaagtccc accgcaagtg gtgaaacttc |
| 3481 | cgtctgcacc tcagactatc cagcccgagg taaaagaagt tgcacctgtc tatgaggctg |
| 3541 | ataccgaaca gacacagaat gttactgtta aacctaagag gttacgcaaa aagcgtaatg |
| 3601 | ttgacccttt gtccaatttt gaacataagg ttattacaga gtgcgttacc atagttttag |
| 3661 | gtgacgcaat tcaagtagcc aagtgctatg gggagtctgt gttagttaat gctgctaaca |
| 3721 | cacatcttaa gcatggcggt ggtatcgctg gtgctattaa tgcggcttca aaaggggctg |
| 3781 | tccaaaaaga gtcagatgag tatattctgg ctaaagggcc gttacaagta ggagattcag |
| 3841 | ttctcttgca aggccattct ctagctaaga atatcctgca tgtcgtaggc ccagatgccc |
| 3901 | gcgctaaaca ggatgtttct ctccttagta agtgctataa ggctatgaat gcatatcctc |
| 3961 | ttgtagtcac tcctcttgtt tcagcaggca tatttggtgt aaaaccagct gtgtcttttg |
| 4021 | attatcttat tagggaggct aagactagag ttttagtcgt cgttaattcc caagatgtct |
| 4081 | ataagagtct taccatagtt gacattccac agagtttgac tttttcatat gatgggttac |
| 4141 | gtggcgcaat acgtaaagct aaagattatg gttttactgt ttttgtgtgc acagacaact |
| 4201 | ctgctaacac taaagttctt aggaacaagg gtgttgatta tactaagaag tttcttacag |
| 4261 | ttgacggtgt gcaatattat tgctacacgt ctaaggacac tttagatgat atcttacaac |
| 4321 | aggctaataa gtctgttggt attatatcta tgcctttggg atatgtgtct catggtttag |
| 4381 | acttaatgca agcagggagt gtcgtgcgta gagttaacgt gccctacgtg tgtctcctag |
| 4441 | ctaataaaga gcaagaagct attttgatgt ctgaagacgt taagttaaac ccttcagaag |
| 4501 | attttataaa gcacgtccgc actaatggtg gttacaattc ttggcattta gtcgagggtg |
| 4561 | aactattggt gcaagactta cgcttaaata agctcctgca ttggtctgat caaaccatat |
| 4621 | gctacaagga tagtgtgttt tatgttgtaa agaatagtac agcttttcca tttgaaacac |
| 4681 | tttcagcatg tcgtgcgtat ttggattcac gcacgacaca gcagttaaca atcgaagtct |
| 4741 | tagtgactgt cgatggtgta aattttagaa cagtcgttct aaataataag aacacttata |
| 4801 | gatcacagct tggatgcgtt ttctttaatg gtgctgatat ttctgacacc attcctgatg |
| 4861 | agaaacagaa tggtcacagt ttatatctag cagacaattt gactgctgat gaaacaaagg |
| 4921 | cgcttaaaga gttatatggc cccgttgatc ctactttctt acacagattc tattcactta |
| 4981 | aggctgcagt ccatgggtgg aagatggttg tgtgtgataa ggtacgttct ctcaaattga |
| 5041 | gtgataataa ttgttatctt aatgcagtta ttatgacact tgatttattg aaggacatta |
| 5101 | aatttgttat acctgctcta cagcatgcat ttatgaaaca taagggcggt gattcaactg |
| 5161 | acttcatagc cctcattatg gcttatggca attgcacatt tggtgctcca gatgatgcct |
| 5221 | ctcggttact tcataccgtg cttgcaaagg ctgagttatg ctgttctgca cgcatggttt |
| 5281 | ggagagagtg gtgcaatgtc tgtggcataa aagatgttgt tctacaaggc ttaaaagctt |
| 5341 | gttgttacgt gggtgtgcaa actgttgaag atctgcgtgc tcgcatgaca tatgtatgcc |
| 5401 | agtgtggtgg tgaacgtcat cggcaattag tcgaacacac caccccctgg ttgctgctct |
| 5461 | caggcacacc aaatgaaaaa ttggtgacaa cctccacggc gcctgatttt gtagcattta |
| 5521 | atgtctttca gggcattgaa acggctgttg gccattatgt tcatgctcgc ctgaagggtg |
| 5581 | gtcttatttt aaagtttgac tctggcaccg ttagcaagac ttcagactgg aagtgcaagg |
| 5641 | tgacagatgt acttttcccc ggccaaaaat acagtagcga ttgtaatgtc gtacggtatt |
| 5701 | ctttggacgg taatttcaga acagaggttg atcccgacct atctgctttc tatgttaagg |
| 5761 | atggtaaata ctttacaagt gaaccacccg taacatattc accagctaca attttagctg |
| 5821 | gtagtgtcta cactaatagc tgccttgtat cgtctgatgg acaacctggc ggtgatgcta |
| 5881 | ttagtttgag ttttaataac cttttagggt ttgattctag taaaccagtc actaagaaat |
| 5941 | acacttactc cttcttgcct aaagaagacg gcgatgtgtt gttggctgag tttgacactt |
| 6001 | atgaccctat ttataagaat ggtgccatgt ataaaggcaa accaattctt tgggtcaata |
| 6061 | aagcatctta tgatactaat cttaataagt tcaatagagc tagtttgcgt caaatttttg |
| 6121 | acgtagcccc cattgaactc gaaaataaat tcacaccttt gagtgtggag tctacaccag |
| 6181 | ttgaacctcc aactgtagat gtggtagcac ttcaacagga aatgacaatt gtcaaatgta |
| 6241 | agggtttaaa taaacctttc gtgaaggaca atgtcagttt cgttgctgat gattcaggta |
| 6301 | ctcccgttgt tgagtatctg tctaaagaag acctacatac attgtatgta gaccctaagt |
| 6361 | atcaagtcat tgtcttaaaa gacaatgtac tttcttctat gcttagattg cacaccgttg |
| 6421 | agtcaggtga tattaacgtt gttgcagctt ccggatcttt gacacgtaaa gtgaagttac |
| 6481 | tatttagggc ttcattttat ttcaaagaat ttgctacccg cactttcact gctaccactg |
| 6541 | ctgtaggtag ttgtataaag agtgtagtgc ggcatctagg tgttactaaa ggcatattga |
| 6601 | caggctgttt tagttttgcc aagatgttat ttatgcttcc actagcttac tttagtgatt |
| 6661 | caaaactcgg caccacagag gttaaagtga gtgctttgaa aacagccggc gttgtgacag |
| 6721 | gtaatgttgt aaaacagtgt tgcactgctg ctgttgattt aagtatggat aagttgcgcc |
| 6781 | gtgtggattg gaaatcaacc ctacggttgt tacttatgtt atgcacaact atggtattgt |
| 6841 | tgtcttctgt gtatcacttg tatgtcttca atcaggtctt atcaagtgat gttatgtttg |
| 6901 | aagatgccca aggtttgaaa aagttctaca aagaagttag agcttaccta ggaatctctt |
| 6961 | ctgcttgtga cggtcttgct tcagcttata gggcgaattc ctttgatgta cctacattct |
| 7021 | gcgcaaaccg ttctgcaatg tgtaattggt gcttgattag ccaagattcc ataactcact |
| 7081 | acccagctct taagatggtt caaacacatc ttagccacta tgttcttaac atagattggt |
| 7141 | tgtggtttgc atttgagact ggtttggcat acatgctcta tacctcggcc ttcaactggt |
| 7201 | tgttgttggc aggtacattg cattatttct ttgcacagac ttccatattt gtagactggc |
| 7261 | ggtcatacaa ttatgctgtg tctagtgcct tctggttatt cacccacatt ccaatggcgg |
| 7321 | gtttggtacg aatgtataat ttgttagcat gcctttggct tttacgcaag ttttatcagc |
| 7381 | atgtaatcaa tggttgcaaa gatacggcat gcttgctctg ctataagagg aaccgactta |
| 7441 | ctagagttga agcttctacc gttgtctgtg gtggaaaacg tacgttttat atcacagcaa |
| 7501 | atggcggtat ttcattctgt cgtaggcata attggaattg tgtggattgt gacactgcag |
| 7561 | gtgtggggaa taccttcatc tgtgaagaag tcgcaaatga cctcactacc gccctacgca |
| 7621 | ggcctattaa cgctacggat agatcacatt attatgtgga ttccgttaca gttaaagaga |
| 7681 | ctgttgttca gtttaattat cgtagagacg gtcaaccatt ctacgagcgg tttcccctct |
| 7741 | gcgcttttac aaatctagat aagttgaagt tcaaagaggt ctgtaaaact actactggta |
| 7801 | tacctgaata caactttatc atctacgact catcagatcg tggccaggaa agtttagcta |
| 7861 | ggtctgcatg tgtttattat tctcaagtct tgtgtaaatc aattcttttg gttgactcaa |
| 7921 | gtttggttac ttctgttggt gattctagtg aaatcgccac taaaatgttt gattcctttg |
| 7981 | ttaatagttt cgtctcgctg tataatgtca cacgcgataa gttggaaaaa cttatctcta |
| 8041 | ctgctcgtga tggcgtaagg cgaggcgata acttccatag tgtcttaaca acattcattg |
| 8101 | acgcagcacg aggccccgca ggtgtggagt ctgatgttga gaccaatgaa attgttgact |
| 8161 | ctgtgcagta tgctcataaa catgacatac aaattactaa tgagagctac aataattatg |
| 8221 | taccctcata tgttaaacct gatagtgtgt ctaccagcga tttaggtagt ctcattgatt |
| 8281 | gtaatgcggc ttcagttaac caaattgtct tgcgtaattc taatggtgct tgcatttgga |
| 8341 | acgctgctgc atatatgaaa ctctcggatg cacttaaacg acagattcgc attgcatgcc |
| 8401 | gtaagtgtaa tttagctttc cggttaacca cctcaaagct acgcgctaat gataatatct |
| 8461 | tatcagttag attcactgct aacaaaattg ttggtggtgc tcctacatgg tttaatgcgt |
| 8521 | tgcgtgactt tacgttaaag ggttatgttc ttgctaccat tattgtgttt ctgtgtgctg |
| 8581 | tactgatgta tttgtgttta cctacatttt ctatggcacc tgttgaattt tatgaagacc |
| 8641 | gcatcttgga ctttaaagtt cttgataatg gtatcattag ggatgtaaat cctgatgata |
| 8701 | agtgctttgc taataagcac cggtccttca cacaatggta tcatgagcat gttggtggtg |
| 8761 | tctatgacaa ctctatcaca tgcccattga cagttgcagt aattgctgga gttgctggtg |
| 8821 | ctcgcattcc agacgtacct actacattgg cttgggtgaa caatcagata attttctttg |
| 8881 | tttctcgagt ctttgctaat acaggcagtg tttgctacac tcctatagat gagataccct |
| 8941 | ataagagttt ctctgatagt ggttgcattc ttccatctga gtgcactatg tttagggatg |
| 9001 | cagagggccg tatgacacca tactgccatg atcctactgt tttgcctggg gcttttgcgt |
| 9061 | acagtcagat gaggcctcat gttcgttacg acttgtatga tggtaacatg tttattaaat |
| 9121 | ttcctgaagt agtatttgaa agtacactta ggattactag aactctgtca actcagtact |
| 9181 | gccggttcgg tagttgtgag tatgcacaag agggtgtttg tattaccaca aatggctcgt |
| 9241 | gggccatttt taatgaccac catcttaata gacctggtgt ctattgtggc tctgatttta |
| 9301 | ttgacattgt caggcggtta gcagtatcac tgttccagcc tattacttat ttccaattga |
| 9361 | ctacctcatt ggtcttgggt ataggtttgt gtgcgttcct gactttgctc ttctattata |
| 9421 | ttaataaagt aaaacgtgct tttgcagatt acacccagtg tgctgtaatt gctgttgttg |
| 9481 | ctgctgttct taatagcttg tgcatctgct ttgttacctc tataccattg tgtatagtac |
| 9541 | cttacactgc attgtactat tatgctacat tctattttac taatgagcct gcatttatta |
| 9601 | tgcatgtttc ttggtacatt atgttcgggc ctatcgttcc catatggatg acctgcgtct |
| 9661 | atacagttgc aatgtgcttt agacacttct tctgggtttt agcttatttt agtaagaaac |
| 9721 | atgtagaagt ttttactgat ggtaagctta attgtagttt ccaggacgct gcctctaata |
| 9781 | tctttgttat taacaaggac acttatgcag ctcttagaaa ctctttaact aatgatgcct |
| 9841 | attcacgatt tttggggttg tttaacaagt ataagtactt ctctggtgct atggaaacag |
| 9901 | ccgcttatcg tgaagctgca gcatgtcatc ttgctaaagc cttacaaaca tacagcgaga |
| 9961 | ctggtagtga tcttctttac caaccaccca actgtagcat aacctctggc gtgttgcaaa |
| 10021 | gcggtttggt gaaaatgtca catcccagtg gagatgttga ggcttgtatg gttcaggtta |
| 10081 | cctgcggtag catgactctt aatggtcttt ggcttgacaa cacagtctgg tgcccacgac |
| 10141 | acgtaatgtg cccggctgac cagttgtctg atcctaatta tgatgccttg ttgatttcta |
| 10201 | tgactaatca tagtttcagt gtgcaaaaac acattggcgc tccagcaaac ttgcgtgttg |
| 10261 | ttggtcatgc catgcaaggc actcttttga agttgactgt cgatgttgct aaccctagca |
| 10321 | ctccagccta cacttttaca acagtgaaac ctggcgcagc atttagtgtg ttagcatgct |
| 10381 | ataatggtcg tccgactggt acattcactg ttgtaatgcg ccctaactac acaattaagg |
| 10441 | gttcctttct gtgtggttct tgtggtagtg ttggttacac caaggagggt agtgtgatca |
| 10501 | atttctgtta catgcatcaa atggaacttg ctaatggtac acataccggt tcagcatttg |
| 10561 | atggtactat gtatggtgcc tttatggata aacaagtgca ccaagttcag ttaacagaca |
| 10621 | aatactgcag tgttaatgta gtagcttggc tttacgcagc aatacttaat ggttgcgctt |
| 10681 | ggtttgtaaa acctaatcgc actagtgttg tttcttttaa tgaatgggct cttgccaacc |
| 10741 | aattcactga atttgttggc actcaatccg ttgacatgtt agctgtcaaa acaggcgttg |
| 10801 | ctattgaaca gctgctttat gcgatccaac aactgtatac tgggttccag ggaaagcaaa |
| 10861 | tccttggcag taccatgttg gaagatgaat tcacacctga ggatgttaat atgcagatta |
| 10921 | tgggtgtggt tatgcagagt ggtgtgagaa aagttacata tggtactgcg cattggttgt |
| 10981 | ttgcgaccct tgtctcaacc tatgtgataa tcttacaagc cactaaattt actttgtgga |
| 11041 | actacttgtt tgagactatt cccacacagt tgttcccact cttatttgtg actatggcct |
| 11101 | tcgttatgtt gttggttaaa cacaaacaca cctttttgac acttttcttg ttgcctgtgg |
| 11161 | ctatttgttt gacttatgca aacatagtct acgagcccac tactcccatt tcgtcagcgc |
| 11221 | tgattgcagt tgcaaattgg cttgccccca ctaatgctta tatgcgcact acacatactg |
| 11281 | atattggtgt ctacattagt atgtcacttg tattagtcat tgtagtgaag agattgtaca |
| 11341 | acccatcact ttctaacttt gcgttagcat tgtgcagtgg tgtaatgtgg ttgtacactt |
| 11401 | atagcattgg agaagcctca agccccattg cctatctggt ttttgtcact acactcacta |
| 11461 | gtgattatac gattacagtc tttgttactg tcaaccttgc aaaagtttgc acttatgcca |
| 11521 | tctttgctta ctcaccacag cttacacttg tgtttccgga agtgaagatg atacttttat |
| 11581 | tatacacatg tttaggtttc atgtgtactt gctattttgg tgtcttctct cttttgaacc |
| 11641 | ttaagcttag agcacctatg ggtgtctatg actttaaggt ctcaacacaa gagttcagat |
| 11701 | tcatgactgc taacaatcta actgcaccta gaaattcttg ggaggctatg gctctgaact |
| 11761 | ttaagttaat aggtattggc ggtacacctt gtataaaggt tgctgctatg cagtctaaac |
| 11821 | ttacagatct taaatgcaca tctgtggttc tcctctctgt gctccaacag ttacacttag |
| 11881 | aggctaatag tagggcctgg gctttctgtg ttaaatgcca taatgatata ttggcagcaa |
| 11941 | cagaccccag tgaggctttc gagaaattcg taagtctctt tgctacttta atgacttttt |
| 12001 | ctggtaatgt agatcttgat gcgttagcta gtgatatttt tgacactcct agcgtacttc |
| 12061 | aagctactct ttctgagttt tcacacttag ctacctttgc tgagttggaa gctgcgcaga |
| 12121 | aagcctatca ggaagctatg gactctggtg acacctcacc acaagttctt aaggctttgc |
| 12181 | agaaggctgt taatatagct aaaaacgcct atgagaagga taaggcagtg gcccgtaagt |
| 12241 | tagaacgtat ggctgatcag gctatgactt ctatgtataa gcaagcacgt gctgaagaca |
| 12301 | agaaagcaaa aattgtcagt gctatgcaaa ctatgttgtt tggtatgatt aagaagctcg |
| 12361 | acaacgatgt tcttaatggt atcatttcta acgctaggaa tggttgtata cctcttagtg |
| 12421 | tcatcccact gtgtgcttca aataaacttc gcgttgtaat tcctgacttc accgtctgga |
| 12481 | atcaggtagt cacatatccc tcgcttaact acgctggggc tttgtgggac attacagtta |
| 12541 | taaacaatgt ggacaatgaa attgttaagt cttcagatgt tgtagacagc aatgaaaatt |
| 12601 | taacatggcc acttgtttta gaatgcacta gggcatccac ttctgccgtt aagttgcaaa |
| 12661 | ataatgagat caaaccttca ggtctaaaaa ccatggttgt gtctgcgggt caagagcaaa |
| 12721 | ctaactgtaa tactagttcc ttagcttatt acgaacctgt gcagggtcgt aaaatgctga |
| 12781 | tggctcttct ttctgataat gcctatctca aatgggcgcg tgttgaaggt aaggacggat |
| 12841 | ttgtcagtgt agagctacaa cctccttgca aattcttgat tgcgggacca aaaggacctg |
| 12901 | aaatccgata tctctatttt gttaaaaatc ttaacaacct tcatcgcggg caagtgttag |
| 12961 | ggcacattgc tgcgactgtt agattgcaag ctggttctaa caccgagttt gcctctaatt |
| 13021 | cctcggtgtt gtcacttgtt aacttcaccg ttgatcctca aaaagcttat ctcgatttcg |
| 13081 | tcaatgcggg aggtgcccca ttgacaaatt gtgttaagat gcttactcct aaaactggta |
| 13141 | caggtatagc tatatctgtt aaaccagaga gtacagctga tcaagagact tatggtggag |
| 13201 | cttcagtgtg tctctattgc cgtgcgcata tagaacatcc tgatgtctct ggtgtttgta |
| 13261 | aatataaggg taagtttgtc caaatccctg ctcagtgtgt ccgtgaccct gtgggatttt |
| 13321 | gtttgtcaaa taccccctgt aatgtctgtc aatattggat tggatatggg tgcaattgtg |
| 13381 | actcgcttag gcaagcagca ctgccccaat ctaaagattc caatttttta aacgagtccg |
| 13441 | gggttctatt gtaaatgccc gaatagaacc ctgttcaagt ggtttgtcca ctgatgtcgt |
| 13501 | ctttagggca tttgacatct gcaactataa ggctaaggtt gctggtattg gaaaatacta |
| 13561 | caagactaat acttgtaggt ttgtagaatt agatgaccaa gggcatcatt tagactccta |
| 13621 | ttttgtcgtt aagaggcata ctatggagaa ttatgaacta gagaagcact gttacgactt |
| 13681 | gttacgtgac tgtgatgctg tagctcccca tgatttcttc atctttgatg tagacaaagt |
| 13741 | taaaacacct catattgtac gtcagcgttt aactgagtac actatgatgg atcttgtata |
| 13801 | tgccctgagg cactttgatc aaaatagcga agtgcttaag gctatcttag tgaagtatgg |
| 13861 | ttgctgtgat gttacctact ttgaaaataa actctggttt gattttgttg aaaatcccag |
| 13921 | tgttattggt gtttatcata aacttggaga acgtgtacgc caagctatct taaacactgt |
| 13981 | taaattttgt gaccacatgg tcaaggctgg tttagtcggt gtgctcacac tagacaacca |
| 14041 | ggaccttaat ggcaagtggt atgattttgg tgacttcgta atcactcaac ctggttcagg |
| 14101 | agtagctata gttgatagct actattctta tttgatgcct gtgctctcaa tgaccgattg |
| 14161 | tctggccgct gagacacata gggattgtga ttttaataaa ccactcattg agtggccact |
| 14221 | tactgagtat gattttactg attataaggt acaactcttt gagaagtact ttaaatattg |
| 14281 | ggatcagacg tatcacgcaa attgcgttaa ttgtactgat gaccgttgtg tgttacattg |
| 14341 | tgctaatttc aatgtattgt ttgctatgac catgcctaag acttgtttcg gacccatagt |
| 14401 | ccgaaagatc tttgttgatg gcgtgccatt tgtagtatct tgtggttatc actacaaaga |
| 14461 | attaggttta gtcatgaata tggatgttag tctccataga cataggctct ctcttaagga |
| 14521 | gttgatgatg tatgccgctg atccagccat gcacattgcc tcctctaacg cttttcttga |
| 14581 | tttgaggaca tcatgtttta gtgtcgctgc acttacaact ggtttgactt ttcaaactgt |
| 14641 | gcggcctggc aattttaacc aagacttcta tgatttcgtg gtatctaaag gtttctttaa |
| 14701 | ggagggctct tcagtgacgc tcaaacattt tttctttgct caagatggta atgctgctat |
| 14761 | tacagattat aattactatt cttataatct gcctactatg tgtgacatca aacaaatgtt |
| 14821 | gttctgcatg gaagttgtaa acaagtactt cgaaatctat gacggtggtt gtcttaatgc |
| 14881 | ttctgaagtg gttgttaata atttagacaa gagtgctggc catcctttta ataagtttgg |
| 14941 | caaagctcgt gtctattatg agagcatgtc ttaccaggag caagatgaac tttttgccat |
| 15001 | gacaaagcgt aacgtcattc ctaccatgac tcaaatgaat ctaaaatatg ctattagtgc |
| 15061 | taagaataga gctcgcactg ttgcaggcgt gtccatactt agcacaatga ctaatcgcca |
| 15121 | gtaccatcag aaaatgctta agtccatggc tgcaactcgt ggagcgactt gcgtcattgg |
| 15181 | tactacaaag ttctacggtg gctgggattt catgcttaaa acattgtaca aagatgttga |
| 15241 | taatccgcat cttatgggtt gggattaccc taagtgtgat agagctatgc ctaatatgtg |
| 15301 | tagaatcttc gcttcactca tattagctcg taaacatggc acttgttgta ctacaaggga |
| 15361 | cagattttat cgcttggcaa atgagtgtgc tcaggtgcta agcgaatatg ttctatgtgg |
| 15421 | tggtggttac tacgtcaaac ctggaggtac cagtagcgga gatgccacca ctgcatatgc |
| 15481 | caatagtgtc tttaacattt tgcaggcgac aactgctaat gtcagtgcac ttatgggtgc |
| 15541 | taatggcaac aagattgttg acaaagaagt taaagacatg cagtttgatt tgtatgtcaa |
| 15601 | tgtttacagg agcactagcc cagaccccaa atttgttgat aaatactatg cttttcttaa |
| 15661 | taagcacttt tctatgatga tactgtctga tgacggtgtc gtttgctata atagtgatta |
| 15721 | tgcagctaag ggttacattg ctggaataca gaattttaag gaaacgctgt attatcagaa |
| 15781 | caatgtcttt atgtctgaag ctaaatgctg ggtggaaacc gatctgaaga aagggccaca |
| 15841 | tgaattctgt tcacagcata cgctttatat taaggatggc gacgatggtt acttccttcc |
| 15901 | ttatccagac ccttcaagaa ttttgtctgc cggttgcttt gtagatgata tcgttaagac |
| 15961 | tgacggtaca ctcatggtag agcggtttgt gtctttggct atagatgctt accctctcac |
| 16021 | aaagcatgaa gatatagaat accagaatgt attctgggtc tacttacagt atatagaaaa |
| 16081 | actgtataaa gaccttacag gacacatgct tgacagttat tctgtcatgc tatgtggtga |
| 16141 | taattctgct aagttttggg aagaggcatt ctatagagat ctctatagtt cgcctaccac |
| 16201 | tttgcaggct gtcggttcat gcgttgtatg ccattcacag acttccctac gctgtgggac |
| 16261 | atgcatccgt agaccatttc tctgctgtaa atgctgctat gatcatgtta tagcaactcc |
| 16321 | acataagatg gttttgtctg tttctcctta cgtttgtaat gcccctggtt gtggcgtttc |
| 16381 | agacgttact aagctatatt taggtggtat gagctacttt tgtgtagatc atagacctgt |
| 16441 | gtgtagtttt ccactttgcg ctaatggtct tgtattcggc ttatacaaga atatgtgcac |
| 16501 | aggtagtcct tctatagttg aatttaatag gttggctacc tgtgactgga ctgaaagtgg |
| 16561 | tgattacacc cttgccaata ctacaacaga accactcaaa ctttttgctg ctgagacttt |
| 16621 | acgtgccact gaagaggcgt ctaagcagtc ttatgctatt gccaccatca aagaaattgt |
| 16681 | tggtgagcgc caactattac ttgtgtggga ggctggcaag tccaaaccac cactcaatcg |
| 16741 | taattatgtt tttactggtt atcatataac caaaaatagt aaagtgcagc tcggtgagta |
| 16801 | cattttcgag cgcattgatt atagtgatgc tgtatcctac aagtctagta caacgtataa |
| 16861 | actgactgta ggtgacatct tcgtacttac ctctcactct gtggctacct tgacggcgcc |
| 16921 | cacaattgtg aatcaagaga ggtatgttaa aattactggg ttgtacccaa ccattacggt |
| 16981 | acctgaagag ttcgcaagtc atgttgccaa cttccaaaaa tcaggttata gtaaatatgt |
| 17041 | cactgttcag ggaccacctg gcactggcaa aagtcatttt gctatagggt tagcgattta |
| 17101 | ctaccctaca gcacgtgttg tttatacagc atgttcacac gcagctgttg atgctttgtg |
| 17161 | tgaaaaagct tttaaatatt tgaacattgc taaatgttcc cgtatcattc ctgcaaaggc |
| 17221 | acgtgttgag tgctatgaca ggtttaaagt taatgagaca aattctcaat atttgtttag |
| 17281 | tactattaat gctctaccag aaacttctgc cgatattctg gtggttgatg aggttagtat |
| 17341 | gtgcactaat tatgatcttt caattattaa tgcacgtatt aaagctaagc acattgtcta |
| 17401 | tgtaggagat ccagcacagt tgccagctcc taggactttg ttgactagag gcacattgga |
| 17461 | accagaaaat ttcaatagtg tcactagatt gatgtgtaac ttaggtcctg acatattttt |
| 17521 | aagtatgtgc tacaggtgtc ctaaggaaat agtaagcact gtgagcgctc ttgtctacaa |
| 17581 | taataaattg ttagccaaga aggagctttc aggccagtgc tttaaaatac tctataaggg |
| 17641 | caatgtgacg catgatgcta gctctgccat taatagacca caactcacat ttgtgaagaa |
| 17701 | ttttattact gccaatccgg catggagtaa ggcagtcttt atttcgcctt acaattcaca |
| 17761 | gaatgctgtg tctcgttcaa tgctgggtct taccactcag actgttgatt cctcacaggg |
| 17821 | ttcagaatac cagtacgtta tcttctgtca aacagcagat acggcacatg ctaacaacat |
| 17881 | taacagattt aatgttgcaa tcactcgtgc ccaaaaaggt attctttgtg ttatgacatc |
| 17941 | tcaggcactc tttgagtcct tagagtttac tgaattgtct tttactaatt acaagctcca |
| 18001 | qtctcagatt gtaactggcc tttttaaaga ttqctctaga gaaacttctg gcctctcacc |
| 18061 | tgcttatgca ccaacatatg ttagtgttga tgacaagtat aagacgagtg atgagctttg |
| 18121 | cgtgaatctt aatttacccg caaatgtccc atactctcgt gttatttcca ggatgggctt |
| 18181 | taaactcgat gcaacagttc ctggatatcc taagcttttc attactcgtg aagaggctgt |
| 18241 | aaggcaagtt cgaagctgga taggcttcga tgttgagggt gctcatgctt cccgtaatgc |
| 18301 | atgtggcacc aatgtgcctc tacaattagg attttcaact ggtgtgaact ttgttgttca |
| 18361 | gccagttggt gttgtagaca ctgagtgggg taacatgtta acgggcattg ctgcacgtcc |
| 18421 | tccaccaggt gaacagttta agcacctcgt gcctcttatg cataaggggg ctgcgtggcc |
| 18481 | tattgttaga cgacgtatag tgcaaatgtt gtcagacact ttagacaaat tgtctgatta |
| 18541 | ctgtacgttt gtttgttggg ctcatggctt tgaattaacg tctgcatcat acttttgcaa |
| 18601 | gataggtaag gaacagaagt gttgcatgtg caatagacgc gctgcagcgt actcttcacc |
| 18661 | tctgcaatct tatgcctgct ggactcattc ctgcggttat gattatgtct acaacccttt |
| 18721 | ctttgtcgat gttcaacagt ggggttatgt aggcaatctt gctactaatc acgatcgtta |
| 18781 | ttgctctgtc catcaaggag ctcatgtggc ttctaatgat gcaataatga ctcgttgttt |
| 18841 | agctattcat tcttgtttta tagaacgtgt ggattgggat atagagtatc cttatatctc |
| 18901 | acatgaaaag aaattgaatt cctgttgtag aatcgttgag cgcaacgtcg tacgtgctgc |
| 18961 | tcttcttgcc ggttcatttg acaaagtcta tgatattggc aatcctaaag gaattcctat |
| 19021 | tgttgatgac cctgtggttg attggcatta ttttgatgca cagcccttga ccaggaaggt |
| 19081 | acaacagctt ttctatacag aggacatggc ctcaagattt gctgatgggc tctgcttatt |
| 19141 | ttggaactgt aatgtaccaa aatatcctaa taatgcaatt gtatgcaggt ttgacacacg |
| 19201 | tgtgcattct gagttcaatt tgccaggttg tgatggcggt agtttgtatg ttaacaagca |
| 19261 | cgcttttcat acaccagcat atgatgtgag tgcattccgt gatctgaaac ctttaccatt |
| 19321 | cttttattat tctactacac catgtgaagt gcatggtaat ggtagtatga tagaggatat |
| 19381 | tgattatgta cccctaaaat ctgcagtctg tattacagct tgtaatttag ggggcgctgt |
| 19441 | ttgtaggaag catgctacag agtacagaga gtatatggaa gcatataatc ttgtctctgc |
| 19501 | atcaggtttc cgcctttggt gttataagac ctttgatatt tataatctct ggtctacttt |
| 19561 | tacaaaagtt caaggtttgg aaaacattgc ttttaatgtt gttaaacaag gccattttat |
| 19621 | tggtgttgag ggtgaactac ctgtagctgt agtcaatgat aagatcttca ccaagagtgg |
| 19681 | cgttaatgac atttgtatgt ttgagaataa aaccactttg cctactaata tagcttttga |
| 19741 | actctatgct aagcgtgctg tacgctcgca tcccgatttc aaattgctac acaatttaca |
| 19801 | agcagacatt tgctacaagt tcgtcctttg ggattatgaa cgtagcaata tttatggtac |
| 19861 | tgctactatt ggtgtatgta agtacactga tattgatgtt aattcagctt tgaatatatg |
| 19921 | ttttgacata cgcgataatt gttcattgga gaagttcatg tctactccca atgccatctt |
| 19981 | tatttctgat agaaaaatca agaaataccc ttgtatggta ggtcctgatt atgcttactt |
| 20041 | caatggtgct atcatccgtg atagtgatgt tgttaaacaa ccagtgaagt tctacttgta |
| 20101 | taagaaagtc aataatgagt ttattgatcc tactgagtgt atttacactc agagtcgctc |
| 20161 | ttgtagtgac ttcctacccc tttctgacat ggagaaagac tttctatctt ttgatagtga |
| 20221 | tgttttcatt aagaagtatg gcttggaaaa ctatgctttt gagcacgtag tctatggaga |
| 20281 | cttctctcat actacgttag gcggtcttca cttgcttatt ggtttataca agaagcaaca |
| 20341 | ggaaggtcat attattatgg aagaaatgct aaaaggtagc tcaactattc ataactattt |
| 20401 | tattactgag actaacacag cggcttttaa ggcggtgtgt tctgttatag atttaaagct |
| 20461 | tgacgacttt gttatgattt taaagagtca agaccttggc gtagtatcca aggttgtcaa |
| 20521 | ggttcctatt gacttaacaa tgattgagtt tatgttatgg tgtaaggatg gacaggttca |
| 20581 | aaccttctac cctcgactcc aggcttctgc agattggaaa cctggtcatg caatgccatc |
| 20641 | cctctttaaa gttcaaaatg taaaccttga acgttgtgag cttgctaatt acaagcaatc |
| 20701 | tattcctatg cctcgcggtg tgcacatgaa catcgctaaa tatatgcaat tgtgccagta |
| 20761 | tttaaatact tgcacattag ccgtgcctgc caatatgcgt gttatacatt ttggcgctgg |
| 20821 | ttctgataaa ggtatcgctc ctggtacctc agttttacga cagtggcttc ctacagatgc |
| 20881 | cattattata gataatgatt taaatgagtt cgtgtcagat gctgacataa ctttatttgg |
| 20941 | agattgtgta actgtacgtg tcggccaaca agtggatctt gttatttccg acatgtatga |
| 21001 | tcctactact aagaatgtaa caggtagtaa tgagtcaaag gctttattct ttacttacct |
| 21061 | gtgtaacctc attaataata atcttgctct tggtgggtct gttgctatta aaataacaga |
| 21121 | acactcttgg agcgttgaac tttatgaact tatgggaaaa tttgcttggt ggactgtttt |
| 21181 | ctgcaccaat gcaaatgcat cctcatctga aggattcctc ttaggtatta attacttggg |
| 21241 | tactattaaa gaaaatatag atggtggtgc tatgcacgcc aactatatat tttggagaaa |
| 21301 | ttccactcct atgaatctga gtacttactc actttttgat ttatccaagt ttcaattaaa |
| 21361 | attaaaagga acaccagttc ttcaattaaa ggagagtcaa attaacgaac tcgtaatatc |
| 21421 | tctcctgtcg cagggtaagt tacttatccg tgacaatgat acactcagtg tttctactga |
| 21481 | tgttcttgtt aacacctaca gaaagttacg ttgatgtagg gccagattct gttaagtctg |
| 21541 | cttgtattga ggttgatata caacagactt tctttgataa aacttggcct aggccaattg |
| 21601 | atgtttctaa ggctgacggt attatatacc ctcaaggccg tacatattct aacataacta |
| 21661 | tcacttatca aggtcttttt ccctatcagg gagaccatgg tgatatgtat gtttactctg |
| 21721 | caggacatgc tacaggcaca actccacaaa agttgtttgt agctaactat tctcaggacg |
| 21781 | tcaaacagtt tgctaatggg tttgtcgtcc gtataggagc agctgccaat tccactggca |
| 21841 | ctgttattat tagcccatct accagcgcta ctatacgaaa aatttaccct gcttttatgc |
| 21901 | tgggttcttc agttggtaat ttctcagatg gtaaaatggg ccgcttcttc aatcatactc |
| 21961 | tagttctttt gcccgatgga tgtggcactt tacttagagc tttttattgt attctagagc |
| 22021 | ctcgctctgg aaatcattgt cctgctggca attcctatac ttcttttgcc acttatcaca |
| 22081 | ctcctgcaac agattgttct gatggcaatt acaatcgtaa tgccagtctg aactctttta |
| 22141 | aggagtattt taatttacgt aactgcacct ttatgtacac ttataacatt accgaagatg |
| 22201 | agattttaga gtggtttggc attacacaaa ctgctcaagg tgttcacctc ttctcatctc |
| 22261 | ggtatgttga tttgtacggc ggcaatatgt ttcaatttgc caccttgcct gtttatgata |
| 22321 | ctattaagta ttattctatc attcctcaca gtattcgttc tatccaaagt gatagaaaag |
| 22381 | cttgggctgc cttctacgta tataaacttc aaccgttaac tttcctgttg gatttttctg |
| 22441 | ttgatggtta tatacgcaga gctatagact gtggttttaa tgatttgtca caactccact |
| 22501 | gctcatatga atccttcgat gttgaatctg gagtttattc agtttcgtct ttcgaagcaa |
| 22561 | aaccttctgg ctcagttgtg gaacaggctg aaggtgttga atgtgatttt tcacctcttc |
| 22621 | tgtctggcac acctcctcag gtttataatt tcaagcgttt ggtttttacc aattgcaatt |
| 22681 | ataatcttac caaattgctt tcactttttt ctgtgaatga ttttacttgt agtcaaatat |
| 22741 | ctccagcagc aattgctagc aactgttatt cttcactgat tttggattac ttttcatacc |
| 22801 | cacttagtat gaaatccgat ctcagtgtta gttctgctgg tccaatatcc cagtttaatt |
| 22861 | ataaacagtc cttttctaat cccacatgtt tgattttagc gactgttcct cataacctta |
| 22921 | ctactattac taagcctctt aagtacagct atattaacaa gtgctctcgt cttctttctg |
| 22981 | atgatcgtac tgaagtacct cagttagtga acgctaatca atactcaccc tgtgtatcca |
| 23041 | ttgtcccatc cactgtgtgg gaagacggtg attattatag gaaacaacta tctccacttg |
| 23101 | aaggtggtgg ctggcttgtt gctagtggct caactgttgc catgactgag caattacaga |
| 23161 | tgggctttgg tattacagtt caatatggta cagacaccaa tagtgtttgc cccaagcttg |
| 23221 | aatttgctaa tgacacaaaa attgcctctc aattaggcaa ttgcgtggaa tattccctct |
| 23281 | atggtgtttc gggccgtggt gtttttcaga attgcacagc tgtaggtgtt cgacagcagc |
| 23341 | gctttgttta tgatgcgtac cagaatttag ttggctatta ttctgatgat ggcaactact |
| 23401 | actgtttgcg tgcttgtgtt agtgttcctg tttctgtcat ctatgataaa gaaactaaaa |
| 23461 | cccacgctac tctatttggt agtgttgcat gtgaacacat ttcttctacc atgtctcaat |
| 23521 | actcccgttc tacgcgatca atgcttaaac ggcgagattc tacatatggc ccccttcaga |
| 23581 | cacctgttgg ttgtgtccta ggacttgtta attcctcttt gttcgtagag gactgcaagt |
| 23641 | tgcctcttgg tcaatctctc tgtgctcttc ctgacacacc tagtactctc acacctcgca |
| 23701 | gtgtgcgctc tgttccaggt gaaatgcgct tggcatccat tgcttttaat catcctattc |
| 23761 | aggttgatca acttaatagt agttatttta aattaagtat acccactaat ttttcctttg |
| 23821 | gtgtgactca ggagtacatt cagacaacca ttcagaaagt tactgttgat tgtaaacagt |
| 23881 | acgtttgcaa tggtttccag aagtgtgagc aattactgcg cgagtatggc cagttttgtt |
| 23941 | ccaaaataaa ccaggctctc catggtgcca atttacgcca ggatgattct gtacgtaatt |
| 24001 | tgtttgcgag cgtgaaaagc tctcaatcat ctcctatcat accaggtttt ggaggtgact |
| 24061 | ttaatttgac acttctagaa cctgtttcta tatctactgg cagtcgtagt gcacgtagtg |
| 24121 | ctattgagga tttgctattt gacaaagtca ctatagctga tcctggttat atgcaaggtt |
| 24181 | acgatgattg catgcagcaa ggtccagcat cagctcgtga tcttatttgt gctcaatatg |
| 24241 | tggctggtta caaagtatta cctcctctta tggatgttaa tatggaagcc gcgtatactt |
| 24301 | catctttgct tggcagcata gcaggtgttg gctggactgc tggcttatcc tcctttgctg |
| 24361 | ctattccatt tgcacagagt atcttttata ggttaaacgg tgttggcatt actcaacagg |
| 24421 | ttctttcaga gaaccaaaag cttattgcca ataagtttaa tcaggctctg ggagctatgc |
| 24481 | aaacaggctt cactacaact aatgaagctt ttcagaaggt tcaggatgct gtgaacaaca |
| 24541 | atgcacaggc tctatccaaa ttagctagcg agctatctaa tacttttggt gctatttccg |
| 24601 | cctctattgg agacatcata caacgtcttg atgttctcga acaggacgcc caaatagaca |
| 24661 | gacttattaa tggccgtttg acaacactaa atgcttttgt tgcacagcag cttgttcgtt |
| 24721 | ccgaatcagc tgctctttcc gctcaattgg ctaaagataa agtcaatgag tgtgtcaagg |
| 24781 | cacaatccaa gcgttctgga ttttgcggtc aaggcacaca tatagtgtcc tttgttgtaa |
| 24841 | atgcccctaa tggcctttac ttcatgcatg ttggttatta ccctagcaac cacattgagg |
| 24901 | ttgtttctgc ttatggtctt tgcgatgcag ctaaccctac taattgtata gcccctgtta |
| 24961 | atggctactt tattaaaact aataacacta ggattgttga tgagtggtca tatactggct |
| 25021 | cgtccttcta tgcacctgag cccattacct cccttaatac taagtatgtt gcaccacagg |
| 25081 | tgacatacca aaacatttct actaacctcc ctcctcctct tctcggcaat tccaccggga |
| 25141 | ttgacttcca agatgagttg gatgagtttt tcaaaaatgt tagcaccagt atacctaatt |
| 25201 | ttggttccct aacacagatt aatactacat tactcgatct tacctacgag atgttgtctc |
| 25261 | ttcaacaagt tgttaaagcc cttaatgagt cttacataga ccttaaagag cttggcaatt |
| 25321 | atacttatta caacaaatgg ccgtggtaca tttggcttgg tttcattgct gggcttgttg |
| 25381 | ccttagctct atgcgtcttc ttcatactgt gctgcactgg ttgtggcaca aactgtatgg |
| 25441 | gaaaacttaa gtgtaatcgt tgttgtgata gatacgagga atacgacctc gagccgcata |
| 25501 | aggttcatgt tcactaatta acgaactatt aatgagagtt caaagaccac ccactctctt |
| 25561 | gttagtgttt tcactctctc ttttggtcac tgcatcctca aaacctctct atgtacctga |
| 25621 | gcattgtcag aattattctg gttgcatgct tagggcttgt attaaaactg cccaagctga |
| 25681 | tacagctggt ctttatacaa attttcgaat tgacgtccca tctgcagaat caactggtac |
| 25741 | tcaatcagtt tctgtcgatc ttgagtcaac ttcaactcat gatggtccta ccgaacatgt |
| 25801 | tactagtgtg aatctttttg acgttggtta ctcagttaat taacgaactc tatggattac |
| 25861 | gtgtctctgc ttaatcaaat ttggcagaag taccttaact caccgtatac tacttgtttg |
| 25921 | tacatcccta aacccacagc taagtataca cctttagttg gcacttcatt gcaccctgtg |
| 25981 | ctgtggaact gtcagctatc ctttgctggt tatactgaat ctgctgttaa ttctacaaaa |
| 26041 | gctttggcca aacaggacgc agctcagcga atcgcttggt tgctacataa ggatggagga |
| 26101 | atccctgatg gatgttccct ctacctccgg cactcaagtt tattcgcgca aagcgaggaa |
| 26161 | gaggagccat tctccaacta agaaactgcg ctacgttaag cgtagatttt ctcttctgcg |
| 26221 | ccatgaagac cttagtgtta ttgtccaacc aacacactat gtcagggtta cattttcaga |
| 26281 | ccccaacatg tggtatctac gttcgggtca tcatttacac tcagttcaca attggcttaa |
| 26341 | accttatggc ggccaacctg tttctgagta ccatattact ctagctttgc taaatctcac |
| 26401 | tgatgaagat ttagctagag atttttcacc cattgcgctc tttttgcgca atgtcagatt |
| 26461 | tgagctacat gagttcgcct tgctgcgcaa aactcttgtt cttaatgcat cagagatcta |
| 26521 | ctgtgctaac atacatagat ttaagcctgt gtatagagtt aacacggcaa tccctactat |
| 26581 | taaggattgg cttctcgttc agggattttc cctttaccat agtggcctcc ctttacatat |
| 26641 | gtcaatctct aaattgcatg cactggatga tgttactcgc aattacatca ttacaatgcc |
| 26701 | atgctttaga acttaccctc aacaaatgtt tgttactcct ttggccgtag atgttgtctc |
| 26761 | catacggtct tccaatcagg gtaataaaca aattgttcat tcttatccca ttttacatca |
| 26821 | tccaggattt taacgaacta tggctttctc ggcgtcttta tttaaacccg tccagctagt |
| 26881 | cccagtttct cctgcatttc atcgcattga gtctactgac tctattgttt tcacatacat |
| 26941 | tcctgctagc ggctatgtag ctgctttagc tgtcaatgtg tgtctcattc ccctattatt |
| 27001 | actgctacgt caagatactt gtcgtcgcag cattatcaga actatggttc tctatttcct |
| 27061 | tgttctgtat aactttttat tagccattgt actagtcaat ggtgtacatt atccaactgg |
| 27121 | aagttgcctg atagccttct tagttatcct cataatactt tggtttgtag atagaattcg |
| 27181 | tttctgtctc atgctgaatt cctacattcc actgtttgac atgcgttccc actttattcg |
| 27241 | tgttagtaca gtttcttctc atggtatggt ccctgtaata cacaccaaac cattatttat |
| 27301 | tagaaacttc gatcagcgtt gcagctgttc tcgttgtttt tatttgcact cttccactta |
| 27361 | tatagagtgc acttatatta gccgttttag taagattagc ctagtttctg taactgactt |
| 27421 | ctccttaaac ggcaatgttt ccactgtttt cgtgcctgca acgcgcgatt cagttcctct |
| 27481 | tcacataatc gccccgagct cgcttatcgt ttaagcagct ctgcgctact atgggtcccg |
| 27541 | tgtagaggct aatccattag tctctctttg gacatatgga aaacgaacta tgttaccctt |
| 27601 | tgtccaagaa cgaatagggt tgttcatagt aaactttttc atttttaccg tagtatgtgc |
| 27661 | tataacactc ttggtgtgta tggctttcct tacggctact agattatgtg tgcaatgtat |
| 27721 | gacaggcttc aataccctgt tagttcagcc cgcattatac ttgtataata ctggacgttc |
| 27781 | agtctatgta aaattccagg atagtaaacc ccctctacca cctgacgagt gggtttaacg |
| 27841 | aactccttca taatgtctaa tatgacgcaa ctcactgagg cgcagattat tgccattatt |
| 27901 | aaagactgga actttgcatg gtccctgatc tttctcttaa ttactatcgt actacagtat |
| 27961 | ggatacccat cccgtagtat gactgtctat gtctttaaaa tgtttgtttt atggctccta |
| 28021 | tggccatctt ccatggcgct atcaatattt agcgccgttt atccaattga tctagcttcc |
| 28081 | cagataatct ctggcattgt agcagctgtt tcagctatga tgtggatttc ctactttgtg |
| 28141 | cagagtatcc ggctgtttat gagaactgga tcatggtggt cattcaatcc tgagactaat |
| 28201 | tgccttttga acgttccatt tggtggtaca actgtcgtac gtccactcgt agaggactct |
| 28261 | accagtgtaa ctgctgttgt aaccaatggc cacctcaaaa tggctggcat gcatttcggt |
| 28321 | gcttgtgact acgacagact tcctaatgaa gtcaccgtgg ccaaacccaa tgtgctgatt |
| 28381 | gctttaaaaa tggtgaagcg gcaaagctac ggaactaatt ccggcgttgc catttaccat |
| 28441 | agatataagg caggtaatta caggagtccg cctattacgg cggatattga acttgcattg |
| 28501 | cttcgagctt aggctcttta gtaagagtat cttaattgat tttaacgaat ctcaatttca |
| 28561 | ttgttatggc atcccctgct gcacctcgtg ctgtttcctt tgccgataac aatgatataa |
| 28621 | caaatacaaa cctatctcga ggtagaggac gtaatccaaa accacgagct gcaccaaata |
| 28681 | acactgtctc ttggtacact gggcttaccc aacacgggaa agtccctctt acctttccac |
| 28741 | ctgggcaggg tgtacctctt aatgccaatt ctacccctgc gcaaaatgct gggtattggc |
| 28801 | ggagacagga cagaaaaatt aataccggga atggaattaa gcaactggct cccaggtggt |
| 28861 | acttctacta cactggaact ggacccgaag cagcactccc attccgggct gttaaggatg |
| 28921 | gcatcgtttg ggtccatgaa gatggcgcca ctgatgctcc ttcaactttt gggacgcgga |
| 28981 | accctaacaa tgattcagct attgttacac aattcgcgcc cggtactaag cttcctaaaa |
| 29041 | acttccacat tgaggggact ggaggcaata gtcaatcatc ttcaagagcc tctagcttaa |
| 29101 | gcagaaactc ttccagatct agttcacaag gttcaagatc aggaaactct acccgcggca |
| 29161 | cttctccagg tccatctgga atcggagcag taggaggtga tctactttac cttgatcttc |
| 29221 | tgaacagact acaagccctt gagtctggca aagtaaagca atcgcagcca aaagtaatca |
| 29281 | ctaagaaaga tgctgctgct gctaaaaata agatgcgcca caagcgcact tccaccaaaa |
| 29341 | gtttcaacat ggtgcaagct tttggtcttc gcggaccagg agacctccag ggaaactttg |
| 29401 | gtgatcttca attgaataaa ctcggcactg aggacccacg ttggccccaa attgctgagc |
| 29461 | ttgctcctac agccagtgct tttatgggta tgtcgcaatt taaacttacc catcagaaca |
| 29521 | atgatgatca tggcaaccct gtgtacttcc ttcggtacag tggagccatt aaacttgacc |
| 29581 | caaagaatcc caactacaat aagtggttgg agcttcttga gcaaaatatt gatgcctaca |
| 29641 | aaaccttccc taagaaggaa aagaaacaaa aggcaccaaa agaagaatca acagaccaaa |
| 29701 | tgtctgaacc tccaaaggag cagcgtgtgc aaggtagcat cactcagcgc actcgcaccc |
| 29761 | gtccaagtgt tcagcctggt ccaatgattg atgttaacac tgattagtgt cactcaaagt |
| 29821 | aacaagatcg cggcaatcgt ttgtgtttgg caaccccatc tcaccatcgc ttgtccactc |
| 29881 | ttgcacagaa tggaatcatg ttgtaattac agtgcaataa ggtaattata acccatttaa |
| 29941 | ttgatagcta tgctttatta aagtgtgtag ctgtagagag aatgttaaag actgtcacct |
| 30001 | ctgcttgatt gcaagtgaac agtgcccccc gggaagagct ctacagtgtg aaatgtaaat |
| 30061 | aaaaaatagc tattattcaa ttagattagg ctaattagat gatttgcaaa aaaaaaaaa. |

The disclosed compositions and methods can be further understood through the following numbered paragraphs.
1. A method of treating a subject for a coronavirus infection comprising administering the subject an effective amount of probenecid, a metabolite or analog thereof, or a pharmaceutically acceptable salt thereof.
2. The method of paragraph 1, wherein the method comprises administering the subject probenecid.
3. The method of paragraphs 1 or 2, wherein the probenecid, metabolite or analog thereof, or pharmaceutically acceptable salt thereof is in a pharmaceutical composition further comprising a pharmaceutically acceptable carrier and/or excipient.
4 The method of any one of paragraphs 1-3, wherein the probenecid, metabolite or analog thereof, or pharmaceutically acceptable salt thereof is administered systemically.
5. The method of any one of paragraphs 1-4, wherein the probenecid, metabolite or analog thereof, or pharmaceutically acceptable salt thereof is administered orally, parenterally, topically or mucosally.
6. The method of any one of paragraphs 1-4, wherein the probenecid, metabolite or analog thereof, or pharmaceutically acceptable salt thereof is administered orally.
7. The method of any one of paragraphs 1-4, wherein the probenecid, metabolite or analog thereof, or pharmaceutically acceptable salt thereof is administered to the lungs (e.g., pulmonary administration).
8. The method of any one of paragraphs 1-4, wherein the probenecid, metabolite or analog thereof, or pharmaceutically acceptable salt thereof is administered intranasally.
9. The method of any one of paragraph 1-8, wherein the probenecid, metabolite or analog thereof, or pharmaceutically acceptable salt thereof is administered in an effective amount to reduce viral replication.
10. The method of any one of paragraph 1-9, wherein the probenecid, metabolite or analog thereof, or pharmaceutically acceptable salt thereof is administered in an effective amount to reduce one or more symptoms of disease, disorder, or illness associated with virus.
11. The method of any one of paragraphs 1-10, wherein the symptoms include fever, congestion in the nasal sinuses and/or lungs, runny or stuffy nose, cough, sneezing, sore throat, body aches, fatigue, shortness of breath, chest tightness, wheezing when exhaling, chills, muscle aches, headache, diarrhea, tiredness, nausea, vomiting, and combinations thereof.
12. The method of any one of paragraphs 1-11, wherein the virus is a Severe acute respiratory syndrome-related coronavirus, a Bat Hp-betacoronavirus Zhejiang2013, a Rousettus bat coronavirus GCCDC1, a Rousettus bat coronavirus HKU9, a Eidolon bat coronavirus C704, a Pipistrellus bat coronavirus HKU5, a Tylonycteris bar coronovirus HKU4, a Middle East respiratory syndrome-related coronavirus, a Hedgehog coronavirus, a murine coronavirus, a Human coronavirus HKU1, a China Rattus coronavirus HKU24, a Betacoronavirus 1, a Myodes coronavirus 2JL14, a Human coronavirus NL63, a Human coronavirus 229E, or a Human coronavirus OC43.
13. The method of paragraph 12, wherein the virus is a Severe acute respiratory syndrome-related coronavirus.
14. The method of paragraph 13, wherein the Severe acute respiratory syndrome-related coronavirus is SARS-CoV-2, SARS-CoV, SARS-CoV RaTG13, SARS-CoV PC4-227, or SARS-CoV BtKY72.
15. The method of paragraph 14, wherein the Severe acute respiratory syndrome-related coronavirus is SARS-CoV-2.
16. The method of paragraph 15, wherein the SARS-CoV-2 comprises a genome encoded by a nucleic acid sequence comprising at least 85%, 86%, 87%, 88%, 89%, 90%, 91%, 92%, 93%, 94%, 95%, 96%, 97%, 98%, 99%, or 100% sequence identity SEQ ID NO: 1 or 2.
17. The method of paragraph 14, wherein the Severe acute respiratory syndrome-related coronavirus is SARS-CoV.
18. The method of paragraph 17, wherein the SAR-CoV comprises a genome encoded by a nucleic acid sequence comprising at least 85%, 86%, 87%, 88%, 89%, 90%, 91%, 92%, 93%, 94%, 95%, 96%, 97%, 98%, 99%, or 100% sequence identity SEQ ID NO:3.
19. The method of paragraph 12, wherein the virus is a Middle East respiratory syndrome-related coronavirus.
20. The method of paragraph 19, wherein the Middle East respiratory syndrome-related coronavirus is MERS-CoV.
21. The method of 20, wherein the MERS-CoV comprises a genome encoded by a nucleic acid sequence comprising at least 85%, 86%, 87%, 88%, 89%, 90%, 91%, 92%, 93%, 94%, 95%, 96%, 97%, 98%, 99%, or 100% sequence identity SEQ ID NO:4.
22. The method of any one of paragraphs 1-21, wherein the subject has been exposed to the coronavirus.
23. The method of paragraph 22, wherein the subject is presently suffering from an infection of the coronavirus.
24. The method of paragraph 23, wherein the subject has COVID-19.
25. The method of any one of paragraphs 1-20, wherein the subject has been exposed to the coronavirus, but is asymptomatic.
26. A method of treating a subject for SAR-CoV-2 infection comprising administering the subject an effective amount of probenecid or a pharmaceutically acceptable salt thereof.
27. The method of paragraph 26, wherein the subject has COVID-19.
28. A prophylactic method comprising administering to a subject that has not be exposed to a coronavirus, an effective amount of probenecid, metabolite or analog thereof, or a pharmaceutically acceptable salt thereof, to reduce viral infection in the subject upon exposure to the virus relative to viral infection in the absence of treatment.
29. The method of paragraph 28, wherein the coronavirus is SAR-CoV-2.
30. The method of any one of paragraphs 1-29, wherein the subject is administered 10 mg -1,000 mg or 50 mg - 500 mg of probenecid, a metabolite or analog thereof, or a pharmaceutically acceptable salt thereof 1 to 5 times per day until symptoms are reduced, the infection clears, or a combination thereof.
31. The method of any one of paragraphs 1-30, wherein the subject is treated by pulse dosing.
32. The method of paragraph 31, wherein the pulse dosing comprises a 1-5 bolus doses of 1,000 mg to 5,000 mg probenecid, a metabolite or analog thereof, or a pharmaceutically acceptable salt thereof.
33. The method of paragraph 32, wherein the bolus dose(s) is followed by a drug holiday until the serum levels of the probenecid, metabolite or analog thereof, or a pharmaceutically acceptable salt thereof are about 0.
34. The method of any one of paragraphs 31-33, wherein the probenecid, metabolite or analog thereof, or a pharmaceutically acceptable salt thereof is administered orally or by infusion.
35. A pharmaceutical composition comprising an effective amount of probenecid, metabolite or analog thereof, or a pharmaceutically acceptable salt thereof for use in the method of any one of paragraphs 1-34.

### EXAMPLE

### Example 1: Probenecid Reduces SARS-CoV-2 Plaque Formation In Vitro

### Materials and Methods

### Plaque Reduction Assay

Prophylactic treatment

Vero E6 cells were plated in a 12 well plates at 5×10⁵cells/well and incubated overnight.

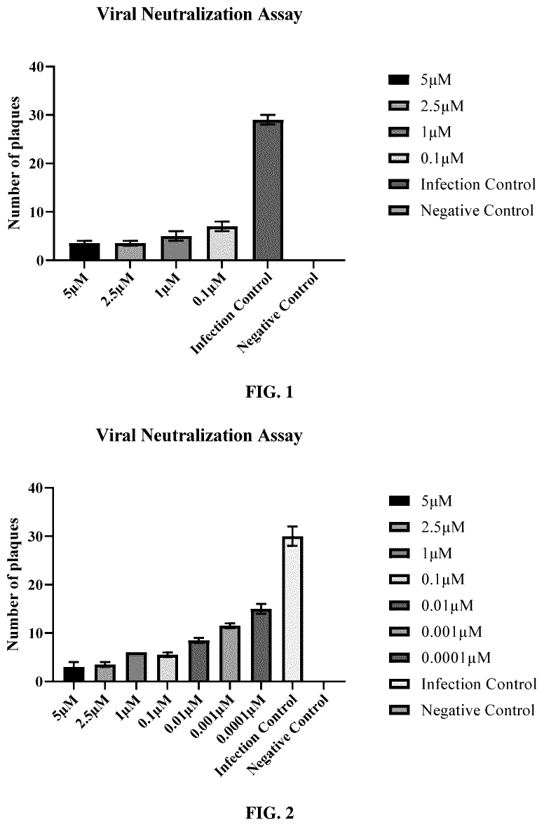

Cells were washed 1× with PBS and probenecid at 0.1 μM, 1 μM, 2.5 μM, or 5 μM was added to the wells in culture media and incubated for 24 hours (FIG. 1). All wells were normalized to 0.05% DMSO. Each concentration was completed in duplicate.

Following pre-treatment, media was discarded and cells were replenished with media containing probenecid (as above) and SARS-CoV-2 (stock grown from Isolate USA-WA1/2020, BEI Resources Catalogue Ref. Number NR-52281). The complete genome of SARS-CoV-2, USA-WA1/2020 has been sequenced (the isolate—GenBank Accession Number: MN985325 and after one passage in Vero cells—GenBank Accession Number: MT020880 and after four passages in Vero cells—GenBank Accession Number: MT246667).

Cells were infected at a MOI of 0.01 for 4 days. Post-infection cells were fixed and stained to visualize plaques. Plaques were quantified manually (FIG. 1).

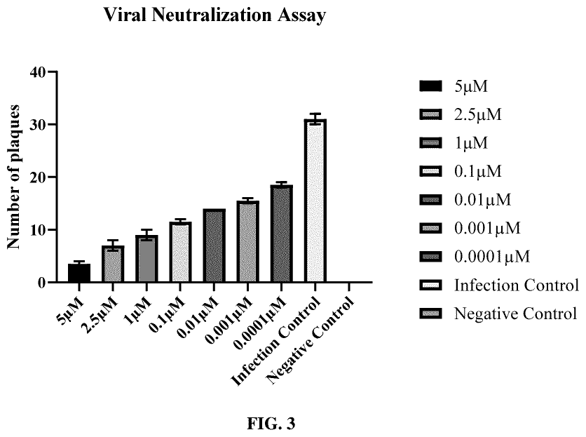

In another experiment, probenecid at 0.0001 μM, 0.001 μM, 0.01 μM, 0.1 μM, 1 μM, 2.5 μM, or 5 μM (FIG. 2) was added. Vero E6 cells were plated in a 6 well plate at 8E5cells/well and incubated overnight. Cells were washed once with PBS and the compound was added to the wells in culture media and incubated for 24 hours. All wells were normalized to 0.05% DMSO. Each concentration was completed in duplicate. Following pre-treatment, media was discarded and cells were replenished with media containing drug (as above) and SARS-CoV-2. Cells were infected at a MOI of 0.01 for 4 days. Post-infection the cells were fixed and stained to visualize plaques. Plaques were quantified (FIG. 2).

### Therapeutic Treatment

Vero E6 cells were plated in a 6 well plate at 8E5cells/well and incubated overnight. Cells were washed once with PBS and infected with virus at a MOI of 0.01 for 1 hour. Following 1 hour infection probenecid at 0.0001 μM, 0.001 μM, 0.01 μM, 0.1 μM, 1 μM, 2.5 μM, or 5 μM was added to the wells in overlay media and incubated 4 days. Post-infection the cells were fixed and stained to visualize plaques. Plaques were quantified (FIG. 3).

### Results

The effect of probenecid on viral replication was investigated using an in vitro plaque formation assay.

Probenecid pre-treatment resulted in a dose dependent reduction in plaque formation for concentrations tested (5 ΞM-0.1 μM) in two independent experiments. 5μM-0.1 μM reduced plaque formation from ˜89% to 72% (FIG. 1), respectively, compared to DMSO treated infected control.

In another experiment, probenecid pre-treatment resulted in a dose dependent reduction in plaque formation for concentrations tested (5 μM-0.0001 μM) in two independent experiments. 5 μM-0.0001 μM reduced plaque formation from ˜93% to 50%, respectively, (FIG. 2), compared to DMSO treated infected control.

In another experiment, probenecid post-treatment resulted in a dose dependent reduction in plaque formation for concentrations tested (5 μM-0.0001 μM). 5 μM-0.0001 μM reduced plaque ˜90% to 40% (FIG. 3), respectively, compared to DMSO treated infected control.

These results show that probenecid significantly reduces viral titer/plaque formation with 24 hour pretreatment at the concentrations tested. These results also show that probenecid significantly reduces viral titer/plaque formation with post-treatment at the concentrations tested.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method of treating a subject for a coronavirus infection comprising administering the subject an effective amount of probenecid or a pharmaceutically acceptable salt thereof.

2. The method of claim 1, wherein the probenecid or pharmaceutically acceptable salt thereof is in a pharmaceutical composition further comprising a pharmaceutically acceptable carrier and/or excipient.

3. The method of claim 1, wherein the probenecid or pharmaceutically acceptable salt thereof is administered systemically.

4. The method of claim 1, wherein the probenecid or pharmaceutically acceptable salt thereof is administered orally, parenterally, topically, or mucosally.

5. The method of claim 4, wherein the probenecid or pharmaceutically acceptable salt thereof is administered mucosally to the lungs, nasal mucosa, or combination thereof.

6. The method of claim 1, wherein the probenecid or pharmaceutically acceptable salt thereof is administered in an effective amount to reduce viral replication.

7. The method of claim 1, wherein the probenecid or pharmaceutically acceptable salt thereof is administered in an effective amount to reduce one or more symptoms of a disease, disorder, or illness associated with the coronavirus.

8. The method of claim 7, wherein the symptoms include fever, congestion in the nasal sinuses and/or lungs, runny or stuffy nose, cough, sneezing, sore throat, body aches, fatigue, shortness of breath, chest tightness, wheezing when exhaling, chills, muscle aches, headache, diarrhea, tiredness, nausea, vomiting, and combinations thereof.

9. The method of claim 1, wherein the coronavirus is a Severe acute respiratory syndrome-related coronavirus, a Bat Hp-betacoronavirus Zhejiang2013, a Rousettus bat coronavirus GCCDC1, a Rousettus bat coronavirus HKU9, a Eidolon bat coronavirus C704, a Pipistrellus bat coronavirus HKU5, a Tylonycteris bar coronovirus HKU4, a Middle East respiratory syndrome-related coronavirus, a Hedgehog coronavirus, a murine coronavirus, a Human coronavirus HKU1, a China Rattus coronavirus HKU24, a Betacoronavirus 1, a Myodes coronavirus 2JL14, a Human coronavirus NL63, a Human coronavirus 229E, or a Human coronavirus OC43.

10. The method of claim 9, wherein the coronavirus is a Severe acute respiratory syndrome-related coronavirus.

11. The method of claim 10, wherein the Severe acute respiratory syndrome-related coronavirus is SARS-CoV-2, SARS-CoV, SARS-CoV RaTG13, SARS-CoV PC4-227, or SARS-CoV BtKY72.

12. The method of claim 11, wherein the Severe acute respiratory syndrome-related coronavirus is SARS-CoV-2.

13. The method of claim 12, wherein the SARS-CoV-2 comprises a genome encoded by a nucleic acid sequence comprising at least 85%, 86%, 87%, 88%, 89%, 90%, 91%, 92%, 93%, 94%, 95%, 96%, 97%, 98%, 99%, or 100% sequence identity SEQ ID NO: 1 or 2.

14. The method of claim 1, wherein the subject is presently suffering from an infection of the coronavirus.

15. The method of claim 14, wherein the subject has COVID-19.

16. The method of claim 1, wherein the subject has been exposed to the coronavirus, but is asymptomatic.

17.-21. (canceled)

22. A method of treating a subject for SAR-CoV-2 infection comprising administering the subject an effective amount of probenecid or a pharmaceutically acceptable salt thereof.

23. The method of claim 22, wherein the subject has COVID-19.

24. (canceled)

25. A method of preventing a coronavirus infection comprising administering to a subject an effective amount of probenecid or a pharmaceutically acceptable salt thereof to reduce coronaviral infection in the subject prior to exposure to the coronavirus.

26. The method of claim 26, wherein the coronavirus is SAR-CoV-2.
